(19) United States
(12) Patent Application Publication
(10) Pub. No.: US 2025/0319130 A1
(43) Pub. Date: Oct. 16, 2025

(54) RECOMBINANT RECEPTORS BINDING B CELL ACTIVATION FACTOR RECEPTOR AND USES THEREOF

(71) Applicant: Seattle Children’s Hospital d/b/a Seattle Children’s Research Institute, Seattle, WA (US)

(72) Inventors: Michael C. Jensen, Bainbridge Island, WA (US); Adam Johnson, Seattle, WA (US); Blake Baxter, Kent, WA (US)

(73) Assignee: Seattle Children’s Hospital d/b/a Seattle Children’s Research Institute, Seattle, WA (US)

(21) Appl. No.: 18/869,984

(22) PCT Filed: Jun. 1, 2023
(86) PCT No.: PCT/US2023/067793

§ 371 Date: Nov. 27, 2024

Related U.S. Application Data
(60) Provisional application No. 63/365,635, filed on Jun. 1, 2022.

(51) Int. Cl. A61K 35/17; A61K 40/11; A61K 40/31; A61K 40/42; A61P 35/00; C07K 14/705; C07K 14/725; C07K 16/28; C12N 5/0783; C12N 15/86

(52) U.S. Cl. CPC A61K 35/17; A61K 40/11; A61K 40/31; A61K 40/4202; A61P 35/00; C07K 14/7051; C07K 14/70517; C07K 14/70521; C07K 14/70578; C07K 16/2878; C12N 5/0636; C12N 15/86; A61K 2239/13; A61K 2239/17; A61K 2239/21; C07K 2317/24; C07K 2317/524; C07K 2317/526; C07K 2317/53; C07K 2317/565; C07K 2317/622; C07K 2319/02; C07K 2319/03; C12N 2510/00; C12N 2740/15043

(57) ABSTRACT

Recombinant receptors with a binding domain that binds B cell activation factor receptor (BAFF-R) are disclosed. Recombinant receptors include chimeric antigen receptors (CAR) having an anti-BAFF-R binding domain, a transmembrane domain, a CD3ζ/4-1BB intracellular signaling domain, and a spacer. Methods and systems to treat BAFF-R-expressing cancers, such as mantle cell lymphoma (MCL), multiple myeloma (MM), acute lymphoblastic leukemia (ALL), and diffuse large B-cell lymphoma (DLBCL), are also provided. The recombinant receptors disclosed herein can bind and elicit cytotoxic effects even in low antigen density conditions.

### CROSS-REFERENCE TO RELATED APPLICATIONS

This application is a U.S National Phase Application based on International Patent Application No. PCT/US2023/067793, filed on Jun. 1, 2023, which claims priority to U.S. Provisional Patent Application No. 63/365,635 filed Jun. 1, 2022, both of which are incorporated herein by reference in their entirety as if fully set forth herein.

### REFERENCE TO SEQUENCE LISTING

The Sequence Listing associated with this application is provided in XML format in lieu of a paper copy and is hereby incorporated by reference into the specification. The name of the file containing the Sequence Listing is 3C47576.XML. The file is 86,016 bytes, was created on Nov. 18, 2024, and is being submitted electronically via Patent Center.

### FIELD OF THE DISCLOSURE

The current disclosure provides recombinant receptors with binding domains that bind anti-B cell activation factor receptor (BAFF-R). The present disclosure also provides methods and systems for using recombinant receptors described herein for therapeutic purposes.

### BACKGROUND OF THE DISCLOSURE

According to the World Health Organization, cancer is a leading cause of death globally, and was responsible for nearly 10 million deaths in 2020.

For many years, the chosen treatments for cancer have been surgery, chemotherapy, and/or radiation therapy. In recent years, more targeted therapies have emerged to specifically target cancer cells by identifying and exploiting specific molecular and/or immunophenotypic changes seen primarily in those cells. For example, many cancer cells preferentially express particular markers on their cellular surfaces and these markers have provided targets for antibody-based therapeutics.

Significant progress has been made in genetically engineering cells of the immune system to target and kill unwanted cell types, such as cancer cells. Many of these immune cells are T cells that have been genetically engineered to express a chimeric antigen receptor (CAR). CAR are proteins including several distinct subcomponents that allow the genetically modified T cells to recognize and kill cancer cells. The subcomponents include at least an extracellular component and an intracellular component expressed as a single protein or assembling into a functional unit. The extracellular component includes a binding domain that binds a marker (e.g., an antigen) that is preferentially present on the surface of unwanted cells. When the binding domain binds such markers, the intracellular component signals the T cell to destroy the bound cell. CAR can additionally include a transmembrane domain that can link the extracellular component to the intracellular component.

Other subcomponents that can increase a CAR's function can also be used. For example, spacers provide CAR with additional conformational flexibility, often increasing the binding domain's ability to bind the targeted cell marker, leading to enhanced cytolytic effects. The appropriate length of a spacer within a particular CAR can depend on numerous factors including how close or far a targeted marker is located from the surface of an unwanted cell's membrane. Thus, while the general structure of CAR are known, the ability of a particular CAR to elicit cytolytic effects in vivo and selection of effective CAR targets remains an area of intense research and investigation. The ability of CAR to elicit cytolytic effects in low antigen density conditions also remains a challenge.

### SUMMARY OF THE DISCLOSURE

One attractive target for cellular immunotherapies is the B-cell activating factor (BAFF) receptor. The BAFF ligand is a critical B cell survival factor that binds three receptors: BAFF-R, TACI, and BCMA20. These receptors are expressed by mature B cells and in a wide range of B cell neoplasms. The current disclosure provides recombinant receptors that bind B-cell activating factor receptor (BAFF-R) for the treatment of BAFF-R-expressing cancers. In particular embodiments, the disclosed recombinant receptor includes, when expressed by a cell (i) an extracellular component including a binding domain that binds BAFF-R and a spacer; (ii) an intracellular component; and (iii) a transmembrane domain linking the extracellular component to the intracellular component.

In particular embodiments, the binding domain is derived from an H90 monoclonal antibody. In particular embodiments, the binding domain that binds BAFF-R includes an scFV including the sequence as set forth in SEQ ID NO: 1 or 6. In particular embodiments, the binding domain that binds BAFF-R includes a variable heavy chain including the sequence as set forth in SEQ ID NO: 8 and a variable light chain including the sequence as set forth in SEQ ID NO: 9. In particular embodiments, the binding domain that binds BAFF-R includes a humanized variable heavy chain including the sequence as set forth in SEQ ID NOs: 32, 8, or 34 and a humanized variable light chain including the sequence as set forth in SEQ ID NOs: 35, 9, or 37.

In particular embodiments, the spacer has a length of 10-15 residues, 110-130 residues, or 230-240 residues. In particular embodiments, the spacer has a length of 12 residues, 119 residues, or 229 residues. In particular embodiments, the spacer includes an IgG4 hinge domain. In particular embodiments, the spacer further includes an IgG4 CH3 domain. In particular embodiments, the spacer further includes an IgG4 CH2 domain. In particular embodiments, the spacer lacks a CD8a hinge domain.

In particular embodiments, the intracellular component includes a CD34 signaling domain or functional portion thereof. In particular embodiments, the intracellular component includes a 4-1BB signaling domain or functional portion thereof. In particular embodiments, the intracellular component includes i) a CD34 signaling domain and ii) a CD27, CD28, 4-1BB, OX-40, CD30, CD40, PD-1, ICOS, LFA-1, CD2, CD7, NKG2C, or B7-H3 signaling domain or functional portions thereof. In particular embodiments, the intracellular component includes a CD34 signaling domain and 4-1BB signaling domain or functional portions thereof.

In particular embodiments, the transmembrane domain includes a CD28 transmembrane domain. In some embodiments, the transmembrane domain lacks a CD8a transmembrane domain.

In particular embodiments, the genetic construct encoding the recombinant receptor can further include or encode a transduction marker, a selection cassette, a self-cleaving polypeptide, a promoter, a suicide switch, or other control features.

Cells genetically modified to express the recombinant receptor disclosed herein can be used in the treatment of BAFF-R-expressing cancers, such as mantle cell lymphoma (MCL), multiple myeloma (MM), acute lymphoblastic leukemia (ALL), and diffuse large B-cell lymphoma (DLBCL).

### BRIEF DESCRIPTION OF THE FIGURES

Some of the drawings submitted herewith may be better understood in color. Applicant considers the color versions of the drawings as part of the original submission and reserves the right to present color images of the drawings in later proceedings.

FIG. 1. Schematic of polynucleotides encoding anti-B cell activation factor receptor (BAFF-R) chimeric antigen receptors (CAR), which include an EF1 promoter; an scFv domain including a VH and VL joined via a linker; a spacer domain selected from either an IgG4-type spacer, such as long length spacer, a medium length spacer, a short length spacer, or a CD8a hinge-type spacer, which also includes a transmembrane domain; a CD28 transmembrane domain or CD8a transmembrane domain, an intracellular signaling domain including a 4-1BB domain and a CD3-zeta domain; a P2A ribosomal skip sequence; a selectable marker, such as a dihydrofolate reductase containing two mutations (DHFRdm); a T2A ribosomal skip sequence; and a cell surface selectable marker, such as a truncated EGFR (EGFRt) polypeptide.

FIG. 2. Schematic of anti-BAFF-R CAR spanning a lipid bilayer, such as a plasma membrane of a cell, including CAR with a CD8a hinge spacer and CD8a transmembrane domain; CAR with short, medium, and long IgG4-type spacers and CD28 transmembrane domains.

FIG. 3. Bar graph of target cell antigen density for BAFF-R showing that endogenous expression of BAFF-R in NALM6, TM-LCL, and Raji cell lines varies, and that K562 cells transduced to express BAFF-R had substantially higher levels of expression compared to other cell lines shown.

FIGS. 4A, 4B. (4A) Proportions of CD8+ T cells or CD4+ T cells expressing anti-BAFF-R CAR. (4B) Flow cytometric analysis of EGFRt sorted, anti-BAFF-R CAR expressing T cells.

FIGS. 5A-5F. Results of a cytotoxicity assay for specific lysis of (5A) control K562, (5B) K562 OKT3, (5C) TM-LCL, (5D) Raji, and (5E) NALM6 target cells by effector cells expressing anti-BAFF-R CAR. X-axis is time (hours). (5F) Bar graph showing percentage lysis of target cells by effector cells expressing anti-BAFF-R CAR.

FIGS. 6A-6C. Bar graph for (6A) interferon gamma (IFNγ), (6B) interleukin-2 (IL-2), or (6C) tumor necrosis factor alpha (TNFα) production by effector cells expressing anti-BAFF-R CAR in the presence of target cells.

FIG. 7. Proportion of gated cells (gated on: lymphocytes/single/live/CD8/EGFRt+) in a series of pie charts (upper panel), and a bar graph (lower right panel) for expression of cytokines (IFNγ, IL-2, TNFα) induced in CD8+ T cells expressing anti-BAFF-R CAR in the presence of target Raji cells.

FIG. 8. Proportion of gated cells (gated on: lymphocytes/single/live/CD8/EGFRt+) in a series of pie carts (upper panel), and a bar graph (lower right panel) for expression of 4-1BB, CD107a, or Nur77 induced in CD8+ T cells expressing anti-BAFF-R CAR in the presence of target Raji cells.

FIGS. 9A-9C (9A) Bioluminescence was measured in mice administered Raji cells expressing reporter genes: mCherry and luciferase, and treated with T cells expressing anti-BAFF-R CAR. The bioluminescence for each day post tumor inoculation is shown for mice not treated with CAR (Mock), anti-BAFF-R CAR with an IgG4 hinge short spacer, anti-BAFF-R CAR with IgG4 hinge medium spacer (IgG4hinge-CH3), anti-BAFF-R CAR with IgG4 hinge long spacer (IgG4hinge-CH2-CH3), and anti-BAFF-R CAR with a CD8a hinge spacer. (9B) Average bioluminescence for the results shown in FIG. 9A. (9C) Percentage survival and tumor related death for mice treated as described for FIG. 9A.

FIG. 10. Sequences supporting the disclosure including Linker (SEQ ID NO: 10), Long (L) spacer (SEQ ID NO: 11), Long (L) spacer encoding sequence (SEQ ID NO: 12), Medium (M) spacer (SEQ ID NO: 13), Medium (M) spacer encoding sequence (SEQ ID NO: 14), Short(S) spacer (SEQ ID NO: 15), Short(S) spacer encoding sequence (SEQ ID NO: 16), CD28Transmembrane Domain (CD28tm) (SEQ ID NOs: 17, 65, and 66), CD28tm encoding sequence (SEQ ID NOs: 18, 67, 68, and 69), CD8a hinge and transmembrane (SEQ ID NO: 19), CD8a hinge domain (spacer) (SEQ ID NO: 70), CD8a transmembrane domain (SEQ ID NO: 71), 41-BB co-stimulatory domain (SEQ ID NOs: 20, 72, and 73), 41-BB co-stimulatory domain encoding sequence (SEQ ID NOs: 21, 74, and 75), CD3Z signaling domain (SEQ ID NO: 22, 76, and 77), CD34 signaling domain encoding sequence (SEQ ID NOs: 23 and 78), 41-BB and CD3Z (SEQ ID NO: 24), P2A (SEQ ID NO: 25), DHFRdm (SEQ ID NO: 26), Thoseaasigna Virus 2A (T2A) Peptide (SEQ ID NO: 27, 79, and 80), T2A encoding sequence (SEQ ID NOs: 28 and 83), Equine Rhinitis A Virus (ERAV) 2A (E2A) Peptide (SEQ ID NO: 81), Foot-And-Mouth Disease Virus 2A (F2A) Peptide (SEQ ID NO: 82), EGFRt (SEQ ID NO: 29 and 84), EGFRt encoding sequence (SEQ ID NO: 30), GMCSFRss (SEQ ID NO: 31), Her2t (SEQ ID NO: 85), and Her2t encoding sequence (SEQ ID NO: 86).

### DETAILED DESCRIPTION

For many years, the chosen treatments for cancer were surgery, chemotherapy, and/or radiation therapy. In recent years, more targeted therapies have emerged to specifically target cancer cells by identifying and exploiting specific molecular and/or immunophenotypic changes seen primarily in those cells. For example, many cancer cells preferentially express particular antigens on their cellular surfaces and these antigens have provided targets for successful therapeutics.

B-cell activating factor receptor (BAFF-R), also known as tumor necrosis factor receptor superfamily member 13C (TNFRSF13C) and BLyS receptor 3 (BR3), is a membrane protein which recognizes B-cell activating factor (BAFF). BAFF is a ligand essential for B cell maturation and survival. BAFF-R plays a role in B-cell (Fu et al. Blood. 2009, 113(19):4627-4636) and T cell (Ye et al. European Journal of Immunology. 2004,34 (10):2750-2759) proliferation and therefore can be associated with malignancies of these cell types. BAFF-R is constitutively saturated in autoimmune and lymphoproliferative diseases (Rodig et al. Human Pathology. 2005, 36(10):1113-1119; Carter et al. Arthritis and Rheumatism. 2005,52(12):3943-3954; and Pers et al. Annals of the New York Academy of Sciences. 2005, 1050:34-39).

The current disclosure provides recombinant receptors that bind BAFF-R for the treatment of BAFF-R-expressing cancers.

In particular embodiments, the disclosed recombinant receptor includes, when expressed by a cell (i) an extracellular component including a binding domain that binds BAFF-R and a spacer; (ii) an intracellular component; and (iii) a transmembrane domain linking the extracellular component to the intracellular component.

In particular embodiments, the binding domain is derived from an H90 monoclonal antibody. In particular embodiments, the binding domain that binds BAFF-R includes an scFV including the sequence as set forth in SEQ ID NO: 1 or 6. In particular embodiments, the binding domain that binds BAFF-R includes a variable heavy chain including the sequence as set forth in SEQ ID NO: 8 and a variable light chain including the sequence as set forth in SEQ ID NO: 9. In particular embodiments, the binding domain that binds BAFF-R includes a humanized variable heavy chain including the sequence as set forth in SEQ ID NOs: 32, 8, or 34 and a humanized variable light chain including the sequence as set forth in SEQ ID NOs: 35, 9, or 37.

In particular embodiments, the spacer has a length of 10-15 residues, 110-130 residues, or 230-240 residues. In particular embodiments, the spacer has a length of 12 residues, 119 residues, or 229 residues. In particular embodiments, the spacer includes an IgG4 hinge domain. In particular embodiments, the spacer further includes an IgG4 CH3 domain. In particular embodiments, the spacer further includes an IgG4 CH2 domain. In particular embodiments, the spacer lacks a CD8a hinge domain.

In particular embodiments, the intracellular component includes a CD3ζ signaling domain or functional portion thereof. In particular embodiments, the intracellular component includes a 4-1BB signaling domain or functional portion thereof. In particular embodiments, the intracellular component includes i) a CD34 signaling domain and ii) a CD27, CD28, 4-1BB, OX-40, CD30, CD40, PD-1, ICOS, LFA-1, CD2, CD7, NKG2C, or B7-H3 signaling domain or functional portions thereof. In particular embodiments, the intracellular component includes a CD3ζ signaling domain and 4-1BB signaling domain or functional portions thereof.

In particular embodiments, the transmembrane domain includes a CD28 transmembrane domain. In some embodiments, the transmembrane domain lacks a CD8a transmembrane domain.

In particular embodiments, the genetic construct encoding the recombinant receptor can further include or encode a selection cassette, a transduction marker, a self-cleaving polypeptide, a promoter, a suicide switch, or other control features.

Cells genetically modified to express the recombinant receptor disclosed herein can be used in the treatment of BAFF-R-expressing cancers, such as mantle cell lymphoma (MCL), multiple myeloma (MM), acute lymphoblastic leukemia (ALL), and diffuse large B-cell lymphoma (DLBCL). In particular embodiments, the recombinant receptors provide cytolytic activity even in low antigen density conditions. For example, data presented herein shows specific lysis in Raji cells which have lower antigen density per cell (6,949 antigens/cell). This is in comparison to the K562 BAFF-R, NALM 6 Parental, and TM-LCL Parental cell lines which express 1,608,470 antigens per cell, 57,504 antigens per cell, and 97,838 antigens per cell, respectively. The ability to provide cytolytic activity in low antigen density conditions provides an important clinical benefit as treatment can be effectively administered before tumor burden expands to generate high antigen density conditions.

As used herein, “low antigen density conditions” refer to a cancer antigen expression level of less than 50,000 antigen molecules per diseased cell, less than 40,000 antigen molecules per diseased cell, less than 30,000 antigen molecules per diseased cell, less than 20,000 antigen molecules per diseased cell, less than 10,000 antigen molecules per diseased cell, or less than 7,000 antigen molecules per diseased cell.

As used herein, “low antigen density conditions” refer to a BAFF-R expression level of less than 50,000 BAFF-R molecules per diseased cell, less than 40,000 BAFF-R molecules per diseased cell, less than 30,000 BAFF-R molecules per diseased cell, less than 20,000 BAFF-R molecules per diseased cell, less than 10,000 BAFF-R molecules per diseased cell, or less than 7,000 BAFF-R molecules per diseased cell.

Aspects of the current disclosure are now described in more supporting detail as follows: (I) Immune Cells; (II) Cell Sample Collection and Cell Enrichment; (III) Genetically Modifying Cell Populations to Express Recombinant Receptors; (III-A) Genetic Engineering Techniques; (III-B) Recombinant Receptors; (III-B-1) Binding Domains; (III-B-2) Intracellular Effector Domains; (III-B-3) Transmembrane Domains; (III-B-4) Spacers, Linkers, and Multimerization Domains; (III-B-5) Control Features Including Tag Cassettes, Transduction Markers, Selection Cassettes, and/or Suicide Switches; (IV) Characterization of Genetically Engineered Cells; (V) Cell Activating Culture Conditions; (VI) Ex Vivo Manufactured Cell Formulations; (VII) Targeted Viral Vectors & Nanoparticles for In Vivo Cell Modification; (VIII) Kits; (IX) Exemplary Embodiments; (X) Experimental Examples; and (XI) Closing Paragraphs. These headings are provided for organizational purposes only and do not limit the scope or interpretation of the disclosure.

(I) Immune Cells. The present disclosure describes cells genetically modified to express a recombinant receptor (e.g., CAR or eTCR). Genetically modified cells can include T cells, B cells, natural killer (NK) cells, NK-T cells, monocytes/macrophages, lymphocytes, hematopoietic stem cells (HSCs), hematopoietic progenitor cells (HPC), and/or a mixture of HSC and HPC (i.e., HSPC). In particular embodiments, genetically modified cells include T cells.

Several different subsets of T cells have been discovered, each with a distinct function. For example, a majority of T cells have a T cell receptor (TCR) existing as a complex of several proteins. The actual T cell receptor is composed of two separate peptide chains, which are produced from the independent T cell receptor alpha and beta (TCRα and TCRβ) genes and are called α- and β-TCR chains.

γδ T cells represent a small subset of T cells that possess a distinct T cell receptor (TCR) on their surface. In γδ T cells, the TCR is made up of one γ-chain and one δ-chain. This group of T cells is much less common (2% of total T cells) than the αβ T cells.

CD3 is expressed on all mature T cells. Activated T cells express 4-1BB (CD137), CD69, and CD25. CD5 and transferrin receptor are also expressed on T cells.

T cells can further be classified into helper cells (CD4+ T cells) and cytotoxic T cells (CTLs, CD8+ T cells), which include cytolytic T cells. T helper cells assist other white blood cells in immunologic processes, including maturation of B cells into plasma cells and activation of cytotoxic T cells and macrophages, among other functions. These cells are also known as CD4+ T cells because they express the CD4 protein on their surface. Helper T cells become activated when they are presented with peptide antigens by MHC class Il molecules that are expressed on the surface of antigen presenting cells (APCs). Once activated, they divide rapidly and secrete small proteins called cytokines that regulate or assist in the active immune response.

Cytotoxic T cells destroy virally infected cells and tumor cells and are also implicated in transplant rejection. These cells are also known as CD8+ T cells because they express the CD8 glycoprotein on their surface. These cells recognize their targets by binding to antigen associated with MHC class I, which is present on the surface of nearly every cell of the body.

“Central memory” T cells (or “TCM”) refers to an antigen experienced CTL that expresses CD62L or CCR7 and CD45RO on the surface thereof and does not express or has decreased expression of CD45RA as compared to naive cells. In particular embodiments, central memory cells are positive for expression of CD62L, CCR7, CD25, CD127, CD45RO, and CD95, and have decreased expression of CD45RA as compared to naive cells.

“Effector memory” T cell (or “TEM”) refers to an antigen experienced T cell that does not express or has decreased expression of CD62L on the surface thereof as compared to central memory cells and does not express or has decreased expression of CD45RA as compared to a naive cell. In particular embodiments, effector memory cells are negative for expression of CD62L and CCR7, compared to naive cells or central memory cells, and have variable expression of CD28 and CD45RA. Effector T cells are positive for granzyme B and perforin as compared to memory or naive T cells.

“Naive” T cells refers to a non-antigen experienced T cell that expresses CD62L and CD45RA and does not express CD45RO as compared to central or effector memory cells. In particular embodiments, naive CD8+ T lymphocytes are characterized by the expression of phenotypic markers of naive T cells including CD62L, CCR7, CD28, CD127, and CD45RA.

Natural killer cells (also known as NK cells, K cells, and killer cells) are activated in response to interferons or macrophage-derived cytokines. They serve to contain viral infections while the adaptive immune response is generating antigen-specific cytotoxic T cells that can clear the infection. NK cells express CD8, CD16 and CD56 but do not express CD3.

NK cells include NK-T cells. NK-T cells are a specialized population of T cells that express a semi invariant T cell receptor (TCR ab) and surface antigens typically associated with natural killer cells. NK-T cells contribute to antibacterial and antiviral immune responses and promote tumor-related immunosurveillance or immunosuppression. Like natural killer cells, NK-T cells can also induce perforin-, Fas-, and TNF-related cytotoxicity. Activated NK-T cells are capable of producing IFN-γ and IL-4. In particular embodiments, NK-T cells are CD3+/CD56+.

Macrophages (and their precursors, monocytes) reside in every tissue of the body (in certain instances as microglia, Kupffer cells and osteoclasts) where they engulf apoptotic cells, pathogens and other non-self-components. Monocytes/macrophages express CD11b, F4/80; CD68; CD11c; IL-4Rα; and/or CD163.

Immature dendritic cells (i.e., pre-activation) engulf antigens and other non-self-components in the periphery and subsequently, in activated form, migrate to T cell areas of lymphoid tissues where they provide antigen presentation to T cells. Dendritic cells express CD1a, CD1b, CD1c, CD1d, CD21, CD35, CD39, CD40, CD86, CD101, CD148, CD209, and DEC-205.

Hematopoietic Stem/Progenitor Cells or HSPC refer to a combination of hematopoietic stem cells and hematopoietic progenitor cells.

Hematopoietic stem cells refer to undifferentiated hematopoietic cells that are capable of self-renewal either in vivo, essentially unlimited propagation in vitro, and capable of differentiation to all other hematopoietic cell types.

A hematopoietic progenitor cell is a cell derived from hematopoietic stem cells or fetal tissue that is capable of further differentiation into mature cell types. In certain embodiments, hematopoietic progenitor cells are CD241^{lo} Lin⁻CD117⁺ hematopoietic progenitor cells. HPC can differentiate into (i) myeloid progenitor cells which ultimately give rise to monocytes and macrophages, neutrophils, basophils, eosinophils, erythrocytes, megakaryocytes/platelets, or dendritic cells; or (ii) lymphoid progenitor cells which ultimately give rise to T cells, B-cells, and NK-cells.

HSPC can be positive for a specific marker expressed in increased levels on HSPC relative to other types of hematopoietic cells. For example, such markers include CD34, CD43, CD45RO, CD45RA, CD59, CD90, CD109, CD117, CD133, CD166, HLA DR, or a combination thereof. Also, the HSPC can be negative for an expressed marker relative to other types of hematopoietic cells. For example, such markers include Lin, CD38, or a combination thereof. In particular embodiments, the HSPC are CD34⁺ cells.

A statement that a cell or population of cells is “positive” for or expressing a particular marker refers to the detectable presence on or in the cell of the particular marker. When referring to a surface marker, the term can refer to the presence of surface expression as detected by flow cytometry, for example, by staining with an antibody that binds to the marker and detecting said antibody, wherein the staining is detectable by flow cytometry at a level substantially above the staining detected carrying out the same procedure with an isotype-matched control under otherwise identical conditions and/or at a level substantially similar to that for cell known to be positive for the marker, and/or at a level substantially higher than that for a cell known to be negative for the marker.

A statement that a cell or population of cells is “negative” for a particular marker or lacks expression of a marker refers to the absence of substantial detectable presence on or in the cell of a particular marker. When referring to a surface marker, the term can refer to the absence of surface expression as detected by flow cytometry, for example, by staining with an antibody that binds to the marker and detecting said antibody, wherein the staining is not detected by flow cytometry at a level substantially above the staining detected carrying out the same procedure with an isotype-matched control under otherwise identical conditions, and/or at a level substantially lower than that for cell known to be positive for the marker, and/or at a level substantially similar as compared to that for a cell known to be negative for the marker.

Cells to be genetically modified according to the teachings of the current disclosure can be patient-derived cells (autologous) or allogeneic when appropriate, and can also be in vivo or ex vivo.

(II) Cell Sample Collection and Cell Enrichment. Methods of sample collection and enrichment are known by those skilled in the art. In particular embodiments, cells are derived from humans, for example a patient to be treated. Cells can be derived from cell lines. The cells in some embodiments are obtained from a xenogeneic source, for example, from mouse, rat, non-human primate, or pig.

In some embodiments, T cells are derived or isolated from samples such as whole blood, peripheral blood mononuclear cells (PBMCs), leukocytes, bone marrow, thymus, tissue biopsy, tumor, lymph node, gut associated lymphoid tissue, mucosa associated lymphoid tissue, spleen, other lymphoid tissues, liver, lung, stomach, intestine, colon, kidney, pancreas, breast, bone, prostate, cervix, testes, ovaries, tonsil, or other organ, and/or cells derived therefrom. In particular embodiments, cells from the circulating blood of a subject are obtained, e.g., by apheresis or leukapheresis. The samples, in particular embodiments, contain lymphocytes, including T cells, monocytes, granulocytes, B cells, other nucleated white blood cells, HSC, HPC, HSPC, red blood cells, and/or platelets, and in some aspects contains cells other than red blood cells and platelets and further processing is necessary. In particular embodiments, T cells are derived from PBMCs.

In some embodiments, blood cells collected from a subject are washed, e.g., to remove the plasma fraction and to place the cells in an appropriate buffer or media for subsequent processing steps. In particular embodiments, the cells are washed with phosphate buffered saline (PBS). In some embodiments, the wash solution lacks calcium and/or magnesium and/or many or all divalent cations. Washing can be accomplished using a semi-automated “flow-through” centrifuge (for example, the Cobe 2991 cell processor, Baxter) according to the manufacturer's instructions. Tangential flow filtration (TFF) can also be performed. In particular embodiments, cells can be re-suspended in a variety of biocompatible buffers after washing, such as, Ca++/Mg++ free PBS.

The isolation can include one or more of various cell preparation and separation steps, including separation based on one or more properties, such as size, density, sensitivity or resistance to particular reagents, and/or affinity, e.g., immunoaffinity, to antibodies or other binding partners. In particular embodiments, the isolation is carried out using the same apparatus or equipment sequentially in a single process stream and/or simultaneously. In particular embodiments, the isolation, culture, and/or engineering of the different populations is carried out from the same starting material, such as from the same sample.

In particular embodiments, a sample can be enriched for T cells by using density-based cell separation methods and related methods. For example, white blood cells can be separated from other cell types in the peripheral blood by lysing red blood cells and centrifuging the sample through a Percoll or Ficoll gradient.

In particular embodiments, a bulk T cell population can be used that has not been enriched for a particular T cell type. In particular embodiments, a selected T cell type can be enriched for and/or isolated based on cell-marker based positive and/or negative selection. In positive selection, cells having bound cellular markers are retained for further use. In negative selection, cells not bound by a capture agent, such as an antibody to a cellular marker are retained for further use. In some examples, both fractions can be retained for a further use. In particular embodiments, CD4+ and/or CD8+ T cells are enriched from PBMCs.

The separation need not result in 100% enrichment or removal of a particular cell population or cells expressing a particular marker. For example, positive selection of or enrichment for cells of a particular type refers to increasing the number or percentage of such cells but need not result in a complete absence of cells not expressing the marker. Likewise, negative selection, removal, or depletion of cells of a particular type refers to decreasing the number or percentage of such cells but need not result in a complete removal of all such cells.

In some examples, multiple rounds of separation steps are carried out, where the positively or negatively selected fraction from one step is subjected to another separation step, such as a subsequent positive or negative selection.

In some embodiments, an antibody or binding domain for a cellular marker is bound to a solid support or matrix, such as a magnetic bead or paramagnetic bead, to allow for separation of cells for positive and/or negative selection. For example, in some embodiments, the cells and cell populations are separated or isolated using immunomagnetic (or affinity magnetic) separation techniques (reviewed in Methods in Molecular Medicine, vol. 58: Metastasis Research Protocols, Vol. 2: Cell Behavior In Vitro and In Vivo, p 17-25 Edited by: S. A. Brooks and U. Schumacher © Humana Press Inc., Totowa, NJ); see also U.S. Pat. No. 4,452,773; U.S. Pat. No. 4,795,698; U.S. Pat. No. 5,200,084; and EP 452342.

In some embodiments, affinity-based selection is via magnetic-activated cell sorting (MACS) (Miltenyi Biotec, Auburn, CA). MACS systems are capable of high-purity selection of cells having magnetized particles attached thereto. In certain embodiments, MACS operates in a mode wherein the non-target and target species are sequentially eluted after the application of the external magnetic field. That is, the cells attached to magnetized particles are held in place while the unattached species are eluted. Then, after this first elution step is completed, the species that were trapped in the magnetic field and were prevented from being eluted are freed in some manner such that they can be eluted and recovered. In certain embodiments, the non-target cells are labelled and depleted from the heterogeneous population of cells.

In some embodiments, a cell population described herein is collected and enriched (or depleted) via flow cytometry, in which cells stained for multiple cell surface markers are carried in a fluidic stream. In some embodiments, a cell population described herein is collected and enriched (or depleted) via preparative scale (FACS)-sorting. In certain embodiments, a cell population described herein is collected and enriched (or depleted) by use of microelectromechanical systems (MEMS) chips in combination with a FACS-based detection system (see, e.g., WO 2010/033140, Cho et al. (2010) Lab Chip 10, 1567-1573; and Godin et al. (2008) J Biophoton. 1(5): 355-376). In both cases, cells can be labeled with multiple markers, allowing for the isolation of well-defined cell subsets at high purity.

Cell-markers for different T cell subpopulations are described above. In particular embodiments, specific subpopulations of T cells, such as cells positive or expressing high levels of one or more surface markers, e.g., CCR7, CD45RO, CD8, CD27, CD28, CD62L, CD127, CD4, and/or CD45RA T cells, are isolated by positive or negative selection techniques.

CD3+, CD28+ T cells can be positively selected for and expanded using anti-CD3/anti-CD28 conjugated magnetic beads (e.g., DYNABEADS® M-450 CD3/CD28 T Cell Expander).

In particular embodiments, a CD8+ or CD4+ selection step is used to separate CD4+ helper and CD8+ cytotoxic T cells. Such CD8+ and CD4+ populations can be further sorted into sub-populations by positive or negative selection for markers expressed or expressed to a relatively higher degree on one or more naive, memory, and/or effector T cell subpopulations.

In some embodiments, enrichment for central memory T (TCM) cells is carried out. In particular embodiments, memory T cells are present in both CD62L subsets of CD8+ peripheral blood lymphocytes. PBMC can be enriched for or depleted of CD62L, CD8 and/or CD62L+CD8+ fractions, such as by using anti-CD8 and anti-CD62L antibodies.

In some embodiments, the enrichment for central memory T (TCM) cells is based on positive or high surface expression of CCR7, CD45RO, CD27, CD62L, CD28, CD3, and/or CD127; in some aspects, it is based on negative selection for cells expressing or highly expressing CD45RA and/or granzyme B. In some aspects, isolation of a CD8+ population enriched for TCM cells is carried out by depletion of cells expressing CD4, CD14, CD45RA, and positive selection or enrichment for cells expressing CCR7, CD45RO, and/or CD62L. In one aspect, enrichment for central memory T (TCM) cells is carried out starting with a negative fraction of cells selected based on CD4 expression, which is subjected to a negative selection based on expression of CD14 and CD45RA, and a positive selection based on CD62L. Such selections in some aspects are carried out simultaneously and in other aspects are carried out sequentially, in either order. In some aspects, the same CD4 expression-based selection step used in preparing the CD8+ cell population or subpopulation, also is used to generate the CD4+ cell population or sub-population, such that both the positive and negative fractions from the CD4-based separation are retained, optionally following one or more further positive or negative selection steps.

Other cell types can be enriched based on known marker profiles and techniques. For example, CD34+ HSC, HSP, and HSPC can be enriched using anti-CD34 antibodies directly or indirectly conjugated to magnetic particles in connection with a magnetic cell separator, for example, the CliniMACS® Cell Separation System (Miltenyi Biotec, Bergisch Gladbach, Germany).

(III) Genetically Modifying Cell Populations to Express Recombinant Receptors. Cell populations can be genetically modified to express recombinant receptors described herein.

(III-A) Genetic Engineering Techniques. Genetic constructs encoding a recombinant receptor disclosed herein can be introduced into cells by any method known in the art, including transfection, electroporation, microinjection, lipofection, calcium phosphate mediated transfection, infection with a viral or bacteriophage vector including the gene sequences, cell fusion, chromosome-mediated gene transfer, microcell-mediated gene transfer, spheroplast fusion, in vivo nanoparticle-mediated delivery, etc. Numerous techniques are known in the art for the introduction of foreign genetic constructs into cells (see e.g., Loeffler and Behr, 1993, Meth. Enzymol. 217:599-618; Cohen, et al., 1993, Meth. Enzymol. 217:618-644; Cline, 1985, Pharmac. Ther. 29:69-92) and may be used, provided that the necessary developmental and physiological functions of the recipient cells are not unduly disrupted. The technique can provide for the stable transfer of the genetic construct to the cell, so that the genetic construct is expressible by the cell and, in certain instances, preferably heritable and expressible by its cell progeny.

The term “gene” refers to a nucleic acid sequence (used interchangeably with polynucleotide or nucleotide sequence) that encodes a recombinant receptor including an anti-BAFF-R binding domain as described herein. This definition includes various sequence polymorphisms, mutations, and/or sequence variants wherein such alterations do not substantially affect the function of the encoded recombinant receptor. The term “gene” may include not only coding sequences but also regulatory regions such as promoters, enhancers, and termination regions. Gene sequences encoding the molecule can be DNA or RNA that directs the expression of the recombinant receptor. These nucleic acid sequences may be a DNA strand sequence that is transcribed into RNA or an RNA sequence that is translated into protein. The sequences can also include degenerate codons of the native sequence or sequences that may be introduced to provide codon preference in a specific cell type (e.g., a mammalian cell). Portions of complete gene sequences are referenced throughout the disclosure as is understood by one of ordinary skill in the art.

Gene sequences encoding a recombinant receptor are provided herein and can also be readily prepared by synthetic or recombinant methods from the relevant amino acid sequences and other description provided herein. In embodiments, the gene sequence encoding any of these sequences can also have one or more restriction enzyme sites at the 5′ and/or 3′ ends of the coding sequence in order to provide for easy excision and replacement of the gene sequence encoding the sequence with another gene sequence encoding a different sequence.

“Encoding” refers to the property of specific sequences of nucleotides in a gene, such as a cDNA, or an mRNA, to serve as templates for synthesis of other macromolecules such as a defined sequence of amino acids. Thus, a gene codes for a protein if transcription and translation of mRNA corresponding to that gene produces the protein in a cell or other biological system. A “gene sequence encoding a protein” includes all nucleotide sequences that are degenerate versions of each other and that code for the same amino acid sequence or amino acid sequences of substantially similar form and function.

Polynucleotide gene sequences encoding more than one portion of an expressed recombinant receptor can be operably linked to each other and relevant regulatory sequences. For example, there can be a functional linkage between a regulatory sequence and an exogenous nucleic acid sequence resulting in expression of the latter. For another example, a first nucleic acid sequence can be operably linked with a second nucleic acid sequence when the first nucleic acid sequence is placed in a functional relationship with the second nucleic acid sequence. For instance, a promoter is operably linked to a coding sequence if the promoter affects the transcription or expression of the coding sequence.

A “vector” is a nucleic acid molecule that is capable of transporting another nucleic acid. Vectors may be, e.g., plasmids (DNA plasmids or RNA plasmids), transposon-based systems, cosmids, bacterial artificial chromosomes, viruses, or phage. An “expression vector” is a vector that is capable of directing the expression of a protein encoded by one or more genes carried by the vector when it is present in the appropriate environment.

“Lentivirus” refers to a genus of retroviruses that are capable of infecting dividing and non-dividing cells. Several examples of lentiviruses include HIV (human immunodeficiency virus: including HIV type 1, and HIV type 2); equine infectious anemia virus; feline immunodeficiency virus (FIV); bovine immune deficiency virus (BIV); and simian immunodeficiency virus (SIV).

A lentiviral vector is a vector derived from at least a portion of a lentivirus genome, including especially a self-inactivating lentiviral vector as provided in Milone et ah, Mol. Ther. 17(8): 1453-1464 (2009). Other examples of lentivirus vectors that may be used in the clinic, include: the LENTIVECTOR® gene delivery technology from Oxford BioMedica, the LENTIMAX™ vector system from Lentigen and the like. Nonclinical types of lentiviral vectors are also available and would be known to one skilled in the art. In particular embodiments, cells are genetically engineered to express a recombinant receptor (e.g., CAR or eTCR) using viral vector, a transposon vector, an integrase vector, or an mRNA vector. In particular embodiments, a viral vector includes a lentiviral vector, a retroviral vector, a foamy viral vector, or a gamma viral vector. In particular embodiments, a viral vector includes a lentiviral vector.

“Retroviruses” are viruses having an RNA genome. “Gammaretrovirus” refers to a genus of the retroviridae family. Exemplary gammaretroviruses include mouse stem cell virus, murine leukemia virus, feline leukemia virus, feline sarcoma virus, and avian reticuloendotheliosis viruses. Foamy viruses are retroviruses useful in delivery of large transgene cassettes.

Retroviral vectors (see Miller, et al., 1993, Meth. Enzymol. 217:581-599) can be used. In such embodiments, the gene to be expressed is cloned into the retroviral vector for its delivery into cells. In particular embodiments, a retroviral vector includes all of the cis-acting sequences necessary for the packaging and integration of the viral genome, i.e., (a) a long terminal repeat (LTR), or portions thereof, at each end of the vector; (b) primer binding sites for negative and positive strand DNA synthesis; and (c) a packaging signal, necessary for the incorporation of genomic RNA into virions. More detail about retroviral vectors can be found in Boesen, et al., 1994, Biotherapy 6:291-302; Clowes, et al., 1994, J. Clin. Invest. 93:644-651; Kiem, et al., 1994, Blood 83:1467-1473; Salmons and Gunzberg, 1993, Human Gene Therapy 4:129-141; and Grossman and Wilson, 1993, Curr. Opin. in Genetics and Devel. 3:110-114. Adenoviruses, adeno-associated viruses (AAV) and alphaviruses can also be used. See Kozarsky and Wilson, 1993, Current Opinion in Genetics and Development 3:499-503, Rosenfeld, et al., 1991, Science 252:431-434; Rosenfeld, et al., 1992, Cell 68:143-155; Mastrangeli, et al., 1993, J. Clin. Invest. 91:225-234; Walsh, et al., 1993, Proc. Soc. Exp. Bioi. Med. 204:289-300; and Lundstrom, 1999, J. Recept. Signal Transduct. Res. 19: 673-686. Other methods of gene delivery include use of mammalian artificial chromosomes (Vos, 1998, Curr. Op. Genet. Dev. 8:351-359); liposomes (Tarahovsky and Ivanitsky, 1998, Biochemistry (Mosc) 63:607-618); ribozymes (Branch and Klotman, 1998, Exp. Nephrol. 6:78-83); and triplex DNA (Chan and Glazer, 1997, J. Mol. Med. 75:267-282).

There are a large number of available viral vectors suitable within the current disclosure, including those identified for human gene therapy applications (see Pfeifer and Verma, 2001, Ann. Rev. Genomics Hum. Genet. 2:177). Methods of using retroviral and lentiviral viral vectors and packaging cells for transducing mammalian host cells with viral particles including recombinant receptor (e.g., CAR) transgenes are described in, e.g., U.S. Pat. No. 8,119,772; Walchli, et al., 2011, PLOS One 6:327930; Zhao, et al., 2005, J. Immunol. 174:4415; Engels, et al., 2003, Hum. Gene Ther. 14:1155; Frecha, et al., 2010, Mol. Ther. 18:1748; and Verhoeyen, et al., 2009, Methods Mol. Biol. 506:97. Retroviral and lentiviral vector constructs and expression systems are also commercially available.

Targeted genetic engineering approaches may also be utilized. The CRISPR (Clustered Regularly Interspaced Short Palindromic Repeats)/Cas (CRISPR-associated protein) nuclease system is an engineered nuclease system used for genetic engineering that is based on a bacterial system. Information regarding CRISPR-Cas systems and components thereof are described in, for example, U.S. Pat. Nos. 8,697,359, 8,771,945, 8,795,965, 8,865,406, 8,871,445, 8,889,356, 8,889,418, 8,895,308, 8,906,616, 8,932,814, 8,945,839, 8,993,233 and 8,999,641 and applications related thereto; and WO2014/018423, WO2014/093595, WO2014/093622, WO2014/093635, WO2014/093655, WO2014/093661, WO2014/093694, WO2014/093701, WO2014/093709, WO2014/093712, WO2014/093718, WO2014/145599, WO2014/204723, WO2014/204724, WO2014/204725, WO2014/204726, WO2014/204727, WO2014/204728, WO2014/204729, WO2015/065964, WO2015/089351, WO2015/089354, WO2015/089364, WO2015/089419, WO2015/089427, WO2015/089462, WO2015/089465, WO2015/089473 and WO2015/089486, WO2016205711, WO2017/106657, WO2017/127807 and applications related thereto.

Particular embodiments utilize zinc finger nucleases (ZFNs) as gene editing agents. ZFNs are a class of site-specific nucleases engineered to bind and cleave DNA at specific positions. ZFNs are used to introduce double stranded breaks (DSBs) at a specific site in a DNA sequence which enables the ZFNs to target unique sequences within a genome in a variety of different cells. A zinc finger is a domain of 30 amino acids within the zinc finger binding domain whose structure is stabilized through coordination of a zinc ion. Examples of zinc fingers include C2H2 zinc fingers, C3H zinc fingers, and C4 zinc fingers. A designed zinc finger domain is a domain not occurring in nature whose design/composition results principally from rational criteria, e.g., application of substitution rules and computerized algorithms for processing information in a database storing information of existing ZFP designs and binding data. A well-known example of a ZFN is a fusion of the Fokl nuclease with a zinc finger DNA binding domain. For additional information regarding ZFNs and ZFNs useful within the teachings of the current disclosure, see, e.g., U.S. Pat. Nos. 6,534,261; 6,607,882; 6,746,838; 6,794,136; 6,824,978; 6,866,997; 6,933,113; 6,979,539; 7,013,219; 7,030,215; 7,220,719; 7,241,573; 7,241,574; 7,585,849; 7,595,376; 6,903,185; 6,479,626; US 2003/0232410 and US 2009/0203140 as well as Gaj et al., Nat Methods, 2012, 9(8):805-7; Ramirez et al., Nucl Acids Res, 2012, 40(12):5560-8; Kim et al., Genome Res, 2012, 22(7): 1327-33; Urnov et al., Nature Reviews Genetics, 2010, 11:636-646; Miller, et al. Nature biotechnology 25, 778-785 (2007); Bibikova, et al. Science 300, 764 (2003); Bibikova, et al. Genetics 161, 1169-1175 (2002); Wolfe, et al. Annual review of biophysics and biomolecular structure 29, 183-212 (2000); Kim, et al. Proceedings of the National Academy of Sciences of the United States of America 93, 1156-1160 (1996); and Miller, et al. The EMBO journal 4, 1609-1614 (1985).

Particular embodiments can use transcription activator like effector nucleases (TALENs) as gene editing agents. TALENs refer to fusion proteins including a transcription activator-like effector (TALE) DNA binding protein and a DNA cleavage domain. TALENs are used to edit genes and genomes by inducing double DSBs in the DNA, which induce repair mechanisms in cells. Generally, two TALENs must bind and flank each side of the target DNA site for the DNA cleavage domain to dimerize and induce a DSB. For additional information regarding TALENs, see U.S. Pat. No. 8,440,431; 8,440,432; 8,450,471; 8,586,363; and 8,697,853; as well as Joung and Sander, Nat Rev Mol Cell Biol, 2013, 14(I):49-55; Beurdeley et al., Nat Commun, 2013, 4:1762; Scharenberg et al., Curr Gene Ther, 2013, 13(4):291-303; Gaj et al., Nat Methods, 2012, 9(8):805-7; Miller, et al. Nature biotechnology 29, 143-148 (2011); Christian, et al. Genetics 186, 757-761 (2010); Boch, et al. Science 326, 1509-1512 (2009); and Moscou, & Bogdanove, Science 326, 1501 (2009).

Particular embodiments can utilize MegaTALs as gene editing agents. MegaTALs have a sc rare-cleaving nuclease structure in which a TALE is fused with the DNA cleavage domain of a meganuclease. Meganucleases, also known as homing endonucleases, are single peptide chains that have both DNA recognition and nuclease function in the same domain. In contrast to the TALEN, the megaTAL only requires the delivery of a single peptide chain for functional activity.

Particular embodiments can use transposon-based systems as gene editing agents to mediate the integration of a recombinant receptor construct into cells. Generally, such methods will involve introducing into cells (i) a first vector encoding a transposase (or a transposase polypeptide) and (ii) a second vector encoding a desired genetic element that is flanked by transposon repeats. Transposons or transposable elements include a (short) nucleic acid sequence with terminal repeat sequences upstream and downstream thereof and encode enzymes that facilitate the excision and insertion of the nucleic acid into target DNA sequences.

Several transposon/transposase systems have been adapted for genetic insertions of heterologous DNA sequences. Examples of such transposases include sleeping beauty (“SB”, e.g., derived from the genome of salmonid fish); piggyback (e.g., derived from lepidopteran cells and/or the Myotis lucifugus); mariner (e.g., derived from Drosophila); frog prince (e.g., derived from Rana pipiens); Tol1; Tol2 (e.g., derived from medaka fish); TcBuster (e.g., derived from the red flour beetle Tribolium castaneum), Helraiser, Himar1, Passport, Minos, Ac/Ds, PIF, Harbinger, Harbinger3-DR, HSmar1, and spinON. Transposases and transposon systems are further described in U.S. Pat. Nos. 6,489,458; 7,148,203; 8,227,432; and 9,228,180.

An integrase can be used to integrate DNA into a host cell. For example, phiC31 integrase is a sequence-specific recombinase encoded within the genome of bacteriophage phiC31. The phiC31 integrase mediates recombination between two sequences of 34 base pairs, one found in phage and the other in a bacterial host, called the attachment site (att). This serine integrase has been shown to function efficiently in many different cell types, including mammalian cells. In the presence of the phiC31 integrase, the attB-containing donor plasmid is unidirectionally integrated into the target genome via recombination at a site that has sequence similarity to the native attP site (called the pseudo-attP site). The phiC31 integrase can integrate plasmids of any size as a single copy and does not require cofactors. The integrated transgene is stably expressed and hereditary.

Nanoparticles that result in selective in vivo genetic modification of targeted cell types have been described and can be used within the teachings of the current disclosure. In particular embodiments, the nanoparticles can be those described in WO2014153114, WO2017181110, and WO201822672.

(III-B) Recombinant Receptors. In particular embodiments, a recombinant receptor is or includes a binding domain that binds a target antigen, wherein the recombinant receptor is expressed by a cell following the artificial introduction of nucleic acid encoding the recombinant receptor into the cell. The recombinant receptor can be, e.g., a CAR, an engineered T cell receptor (eTCR), or a hybrid thereof.

CAR, for example, include several distinct subcomponents that allow genetically modified cells (e.g., T cells) to recognize and kill target cells, such as cancer cells. The subcomponents include at least an extracellular component and an intracellular component. The extracellular component includes a binding domain that binds a marker that is preferentially present on the surface of unwanted cells. When the binding domain binds such markers, the intracellular component activates the cell to destroy the bound cell. CAR can additionally include a transmembrane domain that links the extracellular component to the intracellular component, and other subcomponents that can increase the recombinant receptor's function. For example, the inclusion of a spacer and/or one or more linker sequences can allow the recombinant receptor to have additional conformational flexibility, often increasing the binding domain's ability to bind the targeted cell marker.

(III-B-1) Binding Domains. In certain examples, the current disclosure provides binding domains for use in a recombinant receptor (e.g., CAR) based on antibodies that bind BAFF-R.

BAFF-R also known as tumor necrosis factor receptor superfamily member 13C (TNFRSF13C) is a membrane protein that enhance B-cell survival in vitro and is a regulator of the peripheral B-cell population. In particular embodiments, BAFF-R includes the sequence identified in UniProt reference number Q96RJ3, UniProt reference number Q9D8D0, NCBI reference number GI:16445027, or NCBI reference number GI:16306481 or a variant or homolog having substantial identity thereto.

Antibodies are one example of binding domains and include whole antibodies or binding fragments of an antibody, e.g., Fv, Fab, Fab′, F(ab′)2, and single chain (sc) forms and fragments thereof that bind specifically a cellular marker (such as BAFF-R). Antibodies or antigen binding fragments can include all or a portion of polyclonal antibodies, monoclonal antibodies, human antibodies, humanized antibodies, synthetic antibodies, non-human antibodies, recombinant antibodies, chimeric antibodies, bispecific antibodies, mini bodies, and linear antibodies.

Antibodies are produced from two genes, a heavy chain gene and a light chain gene. Generally, an antibody includes two identical copies of a heavy chain, and two identical copies of a light chain. Within a variable heavy chain and variable light chain, segments referred to as complementary determining regions (CDRs) dictate epitope binding. Each heavy chain has three CDRs (i.e., CDRH1, CDRH2, and CDRH3) and each light chain has three CDRs (i.e., CDRL1, CDRL2, and CDRL3). CDR regions are flanked by framework residues (FR).

The assignment of amino acids to each domain can be in accordance with Kabat numbering (Kabat et al. (1991), “Sequences of Proteins of Immunological Interest,” 5th Ed. Public Health Service, National Institutes of Health, Bethesda, Md. (“Kabat” numbering scheme)); Chothia (Al-Lazikani et al., (1997) JMB 273, 927-948 (“Chothia” numbering scheme)), Martin (Abinandan et al., Mol Immunol. 45:3832-3839 (2008), “Analysis and improvements to Kabat and structurally correct numbering of antibody variable domains”), Gelfand, Contact (MacCallum et al., J. Mol. Biol. 262:732-745 (1996), “Antibody-antigen interactions: Contact analysis and binding site topography,” J. Mol. Biol. 262, 732-745.” (Contact numbering scheme)), IMGT (Lefranc M P et al., “IMGT unique numbering for immunoglobulin and T cell receptor variable domains and Ig superfamily V-like domains,” Dev Comp Immunol, 2003 January; 27(1):55-77 (“IMGT” numbering scheme)), AHo (Honegger A and Plückthun A, “Yet another numbering scheme for immunoglobulin variable domains: an automatic modeling and analysis tool,” J Mol Biol, 2001 Jun. 8; 309(3):657-70, (AHo numbering scheme)), North (North et al., J Mol Biol. 406(2):228-256 (2011), “A new clustering of antibody CDR loop conformations”), or other numbering schemes.

The boundaries of a given CDR or FR may vary depending on the scheme used for identification. For example, the Kabat scheme is based on structural alignments, while the Chothia scheme is based on structural information. Numbering for both the Kabat and Chothia schemes is based upon the most common antibody region sequence lengths, with insertions accommodated by insertion letters, for example, “30a,” and deletions appearing in some antibodies. The two schemes place certain insertions and deletions (“indels”) at different positions, resulting in differential numbering. The Contact scheme is based on analysis of complex crystal structures and is similar in many respects to the Chothia numbering scheme. In particular embodiments, the antibody CDR sequences disclosed herein are according to Kabat numbering. North numbering uses longer sequences in the structural analysis of the conformations of CDR loops. CDR residues can be identified using software programs such as ABodyBuilder.

In some instances, additional scFvs based on the binding domains described herein and for use in a recombinant receptor (e.g., CAR) can be prepared according to methods known in the art (see, for example, Bird et al., (1988) Science 242:423-426 and Huston et al., (1988) Proc. Natl. Acad. Sci. USA 85:5879-5883). ScFv molecules can be produced by linking VH and VL regions of an antibody together using flexible polypeptide linkers. If a short polypeptide linker is employed (e.g., between 5-10 amino acids) intrachain folding is prevented. Interchain folding is also required to bring the two variable regions together to form a functional epitope binding site. For examples of linker orientations and sizes see, e.g., Hollinger et al. 1993 Proc Natl Acad. Sci. U.S.A. 90:6444-6448, US 2005/0100543, US 2005/0175606, US 2007/0014794, and WO2006/020258 and WO2007/024715. More particularly, linker sequences that are used to connect the VL and VH of an scFv are generally five to 35 amino acids in length. In particular embodiments, a VL-VH linker includes from five to 35, ten to 30 amino acids or from 15 to 25 amino acids. Variation in the linker length may retain or enhance activity, giving rise to superior efficacy in activity studies. scFv are commonly used as the binding domains of CAR. In particular embodiments, the recombinant receptor includes a binding domain that binds BAFF-R. In particular embodiments, the binding domain that binds BAFF-R is an scFv. In particular embodiments, the binding domain that binds BAFF-R is an scFV derived from an H90 monoclonal antibody. In particular embodiments, the binding domain that binds BAFF-R includes the sequence:

(SEQ ID NO: 1)
VQLQESGPGLVKPSQTLSLTCTVSGDSITSGYWNWIRQHPGKGLEYIGY
ISYSGSTYYNPSLKSRVTISRDTSKNQYSLKLSSVTAADTAVYYCASPN
YPFYAMDYWGQGTLVTVSSGGGGSGGGGSGGGGSDIVLTQSPATLSLSP
GERATLSCRASESVDNYGISFMNWFQQKPGQAPRLLIYAASNRATGIPA
RFSGSGSGTDFTLTISSLEPEDFAVYYCQQSKEVPWTFGGGTKVEIKR.

In particular embodiments, the binding domain that binds BAFF-R includes the sequence:

(SEQ ID NO: 6)
DIVLTQSPATLSLSPGERATLSCRASESVDNYGISFMNWFQQKPGQAPR
LLIYAASNRATGIPARFSGSGSGTDFTLTISSLEPEDFAVYYCQQSKEV
PWTFGGGTKVEIKRGGGGSGGGGSGGGGSVQLQESGPGLVKPSQTLSLT
CTVSGDSITSGYWNWIRQHPGKGLEYIGYISYSGSTYYNPSLKSRVTIS
RDTSKNQYSLKLSSVTAADTAVYYCASPNYPFYAMDYWGQGTLVTVSS.

In particular embodiments, the binding domain that binds BAFF-R includes a variable heavy chain with complementarity determining regions (CDRH) 1 including the sequence GDSITSGY (SEQ ID NO: 2), a CDRH2 including the sequence ISYSGST (SEQ ID NO: 3), and a CDRH3 including the sequence ASPNYPFYAMDY (SEQ ID NO: 4), and a variable light chain complementarity determining region (CDRL) 1 including the sequence ESVDNYGISF (SEQ ID NO: 5), a CDRL2 including the sequence AAS, and a CDRL3 including the sequence QQSKEVPWT (SEQ ID NO: 7).

In particular embodiments, the binding domain that binds BAFF-R includes a variable heavy chain including the sequence:

(SEQ ID NO: 8)
VQLQESGPGLVKPSQTLSLTCTVSGDSITSGYWNWIRQHPGKGLEYIGY
ISYSGSTYYNPSLKSRVTISRDTSKNQYSLKLSSVTAADTAVYYCASPN
YPFYAMDYWGQGTLVTVSS

and a variable light chain including the sequence:

(SEQ ID NO: 9)
DIVLTQSPATLSLSPGERATLSCRASESVDNYGISFMNWFQQKPGQAPR
LLIYAASNRATGIPARFSGSGSGTDFTLTISSLEPEDFAVYYCQQSKEV
PWTFGGGTKVEIKR.

In particular embodiments, the binding domain that binds BAFF-R includes a humanized VH domain including a sequence including:

(SEQ ID NO: 32)
VQLQESGPGLVKPSQTLSLTCTVSGDSITSGYWNWIRQHPGKGLEYIGY
ISYSGSTYYNPSLKSRVTISRDTSKNQFSLKLSSVTAADTAVYYCASPN
YPFYAMDYWGQGTLVTVSS,
(SEQ ID NO: 8)
VQLQESGPGLVKPSQTLSLTCTVSGDSITSGYWNWIRQHPGKGLEYIGY
ISYSGSTYYNPSLKSRVTISRDTSKNQYSLKLSSVTAADTAVYYCASPN
YPFYAMDYWGQGTLVTVSS,
or
(SEQ ID NO: 34)
VQLQESGPGLVKPSETLSLTCSVSGDSITSGYWNWIRQPPGKGLEYIGY
ISYSGSTYYNPSLKSRVTISRDTSKNQYSLRLSSVTAADTALYYCASPN
YPFYAMDYWGQGTRVTVSS;

and a humanized VL domain including a sequence including:

(SEQ ID NO: 35)
EIVLTQSPATLSLSPGERATLSCRASESVDNYGISFLNWFQQKPGQAPR
LLIYAASNRATGIPARFSGSGSGTDFTLTISSLEPEDFAVYYCQQSKEV
PWTFGGGTKVEIKR,
(SEQ ID NO: 9)
DIVLTQSPATLSLSPGERATLSCRASESVDNYGISFMNWFQQKPGQAPR
LLIYAASNRATGIPARFSGSGSGTDFTLTISSLEPEDFAVYYCQQSKEV
PWTFGGGTKVEIKR,
or
(SEQ ID NO: 37)
DIVMTQSPSSLSASVGDRVTITCRASESVDNYGISFMNWFQQKPGKAPK
LLIYAASNLGSGVPSRFSGSGSGTDFTLTISSLQPEDFATYYCQQSKEV
PWTFGQGTKVEIKR.

Other binding fragments, such as Fv, Fab, Fab′, F(ab′)2, can also be used within the recombinant receptor (e.g., CAR) disclosed herein. Additional examples of antibody-based binding domain formats for use in a recombinant receptor include scFv-based grababodies and soluble VH domain antibodies. These antibodies form binding regions using only heavy chain variable regions. See, for example, Jespers et al., Nat. Biotechnol. 22:1161, 2004; Cortez-Retamozo et al., Cancer Res. 64:2853, 2004; Baral et al., Nature Med. 12:580, 2006; and Barthelemy et al., J. Biol. Chem. 283:3639, 2008.

In particular embodiments, the binding domain includes a humanized antibody or an engineered fragment thereof. In particular embodiments, a non-human antibody is humanized, where one or more amino acid residues of the antibody are modified to increase similarity to an antibody naturally produced in a human or fragment thereof. These nonhuman amino acid residues are often referred to as “import” residues, which are typically taken from an “import” variable domain. As provided herein, humanized antibodies or antibody fragments include one or more CDRs from nonhuman immunoglobulin molecules and framework regions wherein the amino acid residues including the framework are derived completely or mostly from human germline. A humanized antibody can be produced using a variety of techniques known in the art, including CDR-grafting (see, e.g., European Patent No. EP 239,400; WO 91/09967; and U.S. Pat. Nos. 5,225,539, 5,530,101, and 5,585,089), veneering or resurfacing (see, e.g., EP 592,106 and EP 519,596; Padlan, 1991, Molecular Immunology, 28(4/5):489-498; Studnicka et al., 1994, Protein Engineering, 7(6):805-814; and Roguska et al., 1994, PNAS, 91:969-973), chain shuffling (see, e.g., U.S. Pat. No. 5,565,332), and techniques disclosed in, e.g., US 2005/0042664, US 2005/0048617, U.S. Pat. Nos. 6,407,213, 5,766,886, WO 9317105, Tan et al., J. Immunol., 169:1119-25 (2002), Caldas et al., Protein Eng., 13(5):353-60 (2000), Morea et al., Methods, 20(3):267-79 (2000), Baca et al., J. Biol. Chem., 272(16): 10678-84 (1997), Roguska et al., Protein Eng., 9(10):895-904 (1996), Couto et al., Cancer Res., 55 (23 Supp):5973s-5977s (1995), Couto et al., Cancer Res., 55(8):1717-22 (1995), Sandhu J S, Gene, 150(2):409-10 (1994), and Pedersen et al., J. Mol. Biol., 235(3):959-73 (1994). Often, framework residues in the framework regions will be substituted with the corresponding residue from the CDR donor antibody to alter, for example improve, BAFF-R binding. These framework substitutions are identified by methods well-known in the art, e.g., by modeling of the interactions of the CDR and framework residues to identify framework residues important for BAFF-R binding and sequence comparison to identify unusual framework residues at particular positions. (See, e.g., U.S. Pat. No. 5,585,089; and Riechmann et al., 1988, Nature, 332:323).

Functional variants include one or more residue additions or substitutions that do not substantially impact the physiological effects of the protein. Functional fragments include one or more deletions or truncations that do not substantially impact the physiological effects of the protein. A lack of substantial impact can be confirmed by observing experimentally comparable results in an activation study or a binding study. Functional variants and functional fragments of intracellular domains (e.g., intracellular signaling components) transmit activation or inhibition signals comparable to a wild-type reference when in the activated state of the current disclosure. Functional variants and functional fragments of binding domains bind their cognate antigen or ligand at a level comparable to a wild-type reference.

In particular embodiments, a VL region in a binding domain of the present disclosure is derived from or based on a VL of an antibody disclosed herein and contains one or more (e.g., 2, 3, 4, 5, 6, 7, 8, 9, 10) insertions, one or more (e.g., 2, 3, 4, 5, 6, 7, 8, 9, 10) deletions, one or more (e.g., 2, 3, 4, 5, 6, 7, 8, 9, 10) amino acid substitutions (e.g., conservative amino acid substitutions), or a combination of the above-noted changes, when compared with the VL of the antibody disclosed herein. An insertion, deletion or substitution may be anywhere in the VL region, including at the amino- or carboxy-terminus or both ends of this region, provided that each CDR includes zero changes or at most one, two, or three changes and provided a binding domain containing the modified VL region can still specifically bind its target with an affinity similar to the wild type binding domain.

In particular embodiments, a binding domain VH region of the present disclosure can be derived from or based on a VH of an antibody disclosed herein and can contain one or more (e.g., 2, 3, 4, 5, 6, 7, 8, 9, 10) insertions, one or more (e.g., 2, 3, 4, 5, 6, 7, 8, 9, 10) deletions, one or more (e.g., 2, 3, 4, 5, 6, 7, 8, 9, 10) amino acid substitutions (e.g., conservative amino acid substitutions or non-conservative amino acid substitutions), or a combination of the above-noted changes, when compared with the VH of the antibody disclosed herein. An insertion, deletion or substitution may be anywhere in the VH region, including at the amino-or carboxy-terminus or both ends of this region, provided that each CDR includes zero changes or at most one, two, or three changes and provided a binding domain containing the modified VH region can still specifically bind its target with an affinity similar to the wild type binding domain.

In particular embodiments, a binding domain includes or is a sequence that is at least 90%, at least 91%, at least 92%, at least 93%, at least 94%, at least 95%, at least 96%, at least 97%, at least 98%, at least 99%, at least 99.5%, or 100% identical to an amino acid sequence of a light chain variable region (VL) or to a heavy chain variable region (VH), or both, wherein each CDR includes zero changes or at most one, two, or three changes, from an antibody disclosed herein or fragment or derivative thereof that binds to BAFF-R.

(III-B-2) Intracellular Effector Domains. The intracellular effector domains of a recombinant receptor (e.g., CAR) are responsible for activation of the cell in which the recombinant receptor is expressed. The term “effector domain” is thus meant to include any portion of the intracellular domain sufficient to transduce an activation signal. An effector domain can directly or indirectly promote a biological or physiological response in a cell when receiving the appropriate signal. In certain embodiments, an effector domain is part of a protein or protein complex that receives a signal when bound, or it binds directly to a target molecule, which triggers a signal from the effector domain. An effector domain may directly promote a cellular response when it contains one or more signaling domains or motifs, such as an immunoreceptor tyrosine-based activation motif (ITAM). In other embodiments, an effector domain will indirectly promote a cellular response by associating with one or more other proteins that directly promote a cellular response, such as co-stimulatory domains.

Effector domains can provide for activation of at least one function of a modified cell upon binding to the cellular marker expressed by a cancer cell. Activation of the modified cell can include one or more of differentiation, proliferation and/or activation or other effector functions. In particular embodiments, an effector domain can include an intracellular signaling component including a T cell receptor and a co-stimulatory domain which can include the cytoplasmic sequence from co-receptor or co-stimulatory molecule.

An effector domain can include one, two, three or more intracellular signaling components (e.g., receptor signaling domains, cytoplasmic signaling sequences), co-stimulatory domains, or combinations thereof. Exemplary effector domains include signaling and stimulatory domains selected from: 4-1BB (CD137), CARD11, CD3γ, CD3δ, CD3ε, CD3ζ, CD27, CD28, CD79A, CD79B, DAP10, FcRα, FcRβ (FcεR1b), FcRγ, Fyn, HVEM (LIGHTR), ICOS, LAG3, LAT, Lck, LRP, NKG2D, NOTCH1, pTα, PTCH2, OX40, ROR2, Ryk, SLAMF1, Slp76, TCRα, TCRβ, TRIM, Wnt, Zap70, or any combination thereof. In particular embodiments, exemplary effector domains include signaling and co-stimulatory domains selected from: CD86, FcγRIIa, DAP12, CD30, CD40, PD-1, lymphocyte function-associated antigen-1 (LFA-1), CD2, CD7, LIGHT, NKG2C, B7-H3, a ligand that binds CD83, CDS, ICAM-1, GITR, BAFFR, SLAMF7, NKp80 (KLRF1), CD127, CD160, CD19, CD4, CD8α, CD8β, IL2Rβ, IL2Rγ, IL7Rα, ITGA4, VLA1, CD49a, IA4, CD49D, ITGA6, VLA-6, CD49f, ITGAD, CD11d, ITGAE, CD103, ITGAL, CD11a, ITGAM, CD11b, ITGAX, CD11c, ITGB1, CD29, ITGB2, CD18, ITGB7, TNFR2, TRANCE/RANKL, DNAM1 (CD226), SLAMF4 (CD244, 2B4), CD84, CD96 (Tactile), CEACAM1, CRTAM, Ly9 (CD229), PSGL1, CD100 (SEMA4D), CD69, SLAMF6 (NTB-A, Ly108), SLAM (CD150, IPO-3), BLAME (SLAMF8), SELPLG (CD162), LTBR, GADS, PAG/Cbp, NKp44, NKp30, NKp46, TLR1, TLR2, TLR3, TLR4, TLR5, TLR6, TLR7, TLR8, or TLR9. In particular embodiments, the effector domain includes a CD3ζ signaling domain.

Intracellular signaling component sequences that act in a stimulatory manner may include iTAMs. Examples of iTAMs including primary cytoplasmic signaling sequences include those derived from CD3γ, CD3δ, CD3ε, CD3ζ, CD5, CD22, CD66d, CD79a, CD79b, and common FcRγ (FCER1G), FcγRIIa, FcRβ (Fcε Rib), DAP10, and DAP12. In particular embodiments, variants of CD3ζ retain at least one, two, three, or all ITAM regions.

In particular embodiments, an effector domain includes a cytoplasmic portion that associates with a cytoplasmic signaling protein, wherein the cytoplasmic signaling protein is a lymphocyte receptor or signaling domain thereof, a protein including a plurality of ITAMs, a co-stimulatory domain, or any combination thereof.

Additional examples of intracellular signaling components include the cytoplasmic sequences of the CD3ζ chain, and/or co-receptors that act in concert to initiate signal transduction following binding domain engagement.

A co-stimulatory domain is a domain whose activation can be required for an efficient lymphocyte response to cellular marker binding. Some molecules are interchangeable as intracellular signaling components or co-stimulatory domains. Examples of costimulatory domains include CD27, CD28, 4-1BB (CD 137), OX40, CD30, CD40, PD-1, ICOS, lymphocyte function-associated antigen-1 (LFA-1), CD2, CD7, LIGHT, NKG2C, B7-H3, and a ligand that binds CD83. For example, CD27 co-stimulation has been demonstrated to enhance expansion, effector function, and survival of human CAR-T cells in vitro and augments human T cell persistence and anti-cancer activity in vivo (Song et al. Blood. 2012; 119(3):696-706). Further examples of such co-stimulatory domain molecules include CDS, ICAM-1, GITR, BAFFR, HVEM (LIGHTR), SLAMF7, NKp80 (KLRF1), NKp44, NKp30, NKp46, CD160, CD19, CD4, CD8α, CD8β, IL2Rβ, IL2Rγ, IL7Rα, ITGA4, VLA1, CD49a, ITGA4, IA4, CD49D, ITGA6, VLA-6, CD49f, ITGAD, CDIId, ITGAE, CD103, ITGAL, CDIIa, ITGAM, CDI Ib, ITGAX, CDIIc, ITGBI, CD29, ITGB2, CD18, ITGB7, TNFR2, TRANCE/RANKL, DNAM1 (CD226), SLAMF4 (CD244, 2B4), CD84, CD96 (Tactile), NKG2D, CEACAM1, CRTAM, Ly9 (CD229), PSGL1, CD100 (SEMA4D), CD69, SLAMF6 (NTB-A, Lyl08), SLAM (SLAMF1, CD150, IPO-3), BLAME (SLAMF8), SELPLG (CD162), LTBR, LAT, GADS, SLP-76, PAG/Cbp, and CD19a. In particular embodiments, the co-stimulatory domain includes CD27, CD28, 4-1BB, OX-40, CD30, CD40, PD-1, ICOS, LFA-1, CD2, CD7, NKG2C, and/or B7-H3. In particular embodiments, the co-stimulatory domain includes a 4-1BB signaling domain.

In particular embodiments, the nucleic acid sequences encoding the intracellular signaling components includes CD3ζ encoding sequence (SEQ ID NO: 23) and a variant of the 4-1BB signaling encoding sequence (SEQ ID NO: 21). In particular embodiments, the amino acid sequence of the intracellular signaling component includes a variant of CD3ζ (SEQ ID NO: 22) and a portion of the 4-1BB (SEQ ID NO: 20) intracellular signaling component. In particular embodiments, the amino acid sequence including the CD3ζ and 4-1BB intracellular signaling domains is set forth in SEQ ID NO: 24.

In particular embodiments, the intracellular signaling component includes (i) all or a portion of the signaling domain of CD3ζ, (ii) all or a portion of the signaling domain of 4-1BB, or (iii) all or a portion of the signaling domain of CD3ζ and 4-1BB.

Intracellular components may also include one or more of a protein of a Wnt signaling pathway (e.g., LRP, Ryk, or ROR2), NOTCH signaling pathway (e.g., NOTCH1, NOTCH2, NOTCH3, or NOTCH4), Hedgehog signaling pathway (e.g., PTCH or SMO), receptor tyrosine kinases (RTKs) (e.g., epidermal growth factor (EGF) receptor family, fibroblast growth factor (FGF) receptor family, hepatocyte growth factor (HGF) receptor family, insulin receptor (IR) family, platelet-derived growth factor (PDGF) receptor family, vascular endothelial growth factor (VEGF) receptor family, tropomycin receptor kinase (Trk) receptor family, ephrin (Eph) receptor family, AXL receptor family, leukocyte tyrosine kinase (LTK) receptor family, tyrosine kinase with immunoglobulin-like and EGF-like domains 1 (TIE) receptor family, receptor tyrosine kinase-like orphan (ROR) receptor family, discoidin domain (DDR) receptor family, rearranged during transfection (RET) receptor family, tyrosine-protein kinase-like (PTK7) receptor family, related to receptor tyrosine kinase (RYK) receptor family, or muscle specific kinase (MuSK) receptor family); G-protein-coupled receptors, GPCRs (Frizzled or Smoothened); serine/threonine kinase receptors (BMPR or TGFR); or cytokine receptors (IL1R, IL2R, IL7R, or IL15R).

(III-B-3) Transmembrane Domains. As indicated, transmembrane domains within a recombinant receptor (e.g., CAR) serve to connect the extracellular component and intracellular component through the cell membrane. The transmembrane domain can anchor the expressed molecule in the modified cell's membrane.

The transmembrane domain can be derived either from a natural and/or a synthetic source. When the source is natural, the transmembrane domain can be derived from any membrane-bound or transmembrane protein. Transmembrane domains can include at least the transmembrane region(s) of the α, β or ζ chain of a T cell receptor, CD28, CD27, CD3 epsilon, CD45, CD4, CD5, CD8, CD9, CD16, CD22; CD33, CD37, CD64, CD80, CD86, CD134, CD137 CD154, Toll-like receptor 1 (TLR1), TLR2, TLR3, TLR4, TLR5, TLR6, TLR7, TLR8, and TLR9. In particular embodiments, a transmembrane domain may include at least the transmembrane region(s) of, e.g., KIRDS2, OX40, CD2, CD27, LFA-1 (CD 11a, CD18), ICOS (CD278), 4-1BB (CD137), GITR, CD40, BAFFR, HVEM (LIGHTR), SLAMF7, NKp80 (KLRF1), NKp44, NKp30, NKp46, CD160, CD19, IL2Rβ, IL2Rγ, IL7R a, ITGA1, VLA1, CD49a, ITGA4, IA4, CD49D, ITGA6, VLA-6, CD49f, ITGAD, CDI Id, ITGAE, CD103, ITGAL, CDI Ia, ITGAM, CDI Ib, ITGAX, CDI Ic, ITGB1, CD29, ITGB2, CD18, ITGB7, TNFR2, DNAM1(CD226), SLAMF4 (CD244, 2B4), CD84, CD96 (Tactile), CEACAM1, CRT AM, Ly9(CD229), PSGL1, CD100 (SEMA4D), SLAMF6 (NTB-A, Lyl08), SLAM (SLAMF1, CD150, IPO-3), BLAME (SLAMF8), SELPLG (CD162), LTBR, PAG/Cbp, NKG2D, or NKG2C. In particular embodiments, a variety of human hinges can be employed as well including the human Ig (immunoglobulin) hinge (e.g., an IgG4 hinge, an IgD hinge), a GS linker (e.g., a GS linker described herein), a KIR2DS2 hinge or a CD8a hinge. In particular embodiments, the recombinant receptor (e.g., CAR) includes a CD28 transmembrane domain. It has been shown that a CD28 transmembrane domain reduces the antigen-threshold for second-generation 4-1BB CAR T cell activation.

In particular embodiments, a transmembrane domain has a three-dimensional structure that is thermodynamically stable in a cell membrane, and generally ranges in length from 15 to 30 amino acids. The structure of a transmembrane domain can include an α helix, a β barrel, a β sheet, a β helix, or any combination thereof.

A transmembrane domain can include one or more additional amino acids adjacent to the transmembrane region, e.g., one or more amino acid within the extracellular region of the recombinant receptor (e.g., up to 15 amino acids of the extracellular region) and/or one or more additional amino acids within the intracellular region of the recombinant receptor (e.g., up to 15 amino acids of the intracellular components). In one aspect, the transmembrane domain is from the same protein that the signaling domain, co-stimulatory domain or the hinge domain is derived from. In another aspect, the transmembrane domain is not derived from the same protein that any other domain of the recombinant receptor is derived from. In some instances, the transmembrane domain can be selected or modified by amino acid substitution to avoid binding of such domains to the transmembrane domains of the same or different surface membrane proteins to minimize interactions with other unintended members of the receptor complex. In particular embodiments, the transmembrane domain is encoded by the nucleic acid sequence encoding the CD28 transmembrane domain (SEQ ID NO: 18). In particular embodiments, the transmembrane domain includes the amino acid sequence of the CD28 transmembrane domain (SEQ ID NO: 17). In particular embodiments, a sequence including the amino acid sequence of CD8a hinge and transmembrane domain is set forth in SEQ ID NO: 19.

(III-B-4) Spacers, Linkers, and Multimerization Domains. Spacers are used to create appropriate distances and/or flexibility from other recombinant receptor sub-components. As indicated, in particular embodiments, the length of a spacer is customized for binding targeted (BAFF-R-expressing) cells and mediating destruction. In particular embodiments, a spacer length can be selected based upon the location of a cellular marker epitope, affinity of a binding domain for the epitope, and/or the ability of the BAFF-R-binding agent to mediate cell destruction following BAFF-R binding.

Spacers typically include those having 10 to 250 amino acids, 10 to 200 amino acids, 10 to 150 amino acids, 10 to 100 amino acids, 10 to 50 amino acids, or 10 to 25 amino acids.

In particular embodiments, a spacer is 5 amino acids, 8 amino acids, 10 amino acids, 12 amino acids, 14 amino acids, 20 amino acids, 21 amino acids, 26 amino acids, 27 amino acids, 45 amino acids, 50 amino acids, or 75 amino acids. In particular embodiments, the spacer is 10-15 amino acids (or residues) in length. In particular embodiments, the spacer includes a length of 12 amino acids. These lengths qualify as short spacers.

In particular embodiments, a spacer is 76 amino acids, 90 amino acids, 100 amino acids, 110 amino acids, 120 amino acids, 125 amino acids, 128 amino acids, 131 amino acids, 135 amino acids, 140 amino acids, 150 amino acids, 160 amino acids, 170 amino acids, or 179 amino acids. In particular embodiments, the spacer is 110-130 amino acids (or residues) in length. In particular embodiments, the spacer includes a length of 119 amino acids. These lengths qualify as medium spacers.

In particular embodiments, a spacer is 180 amino acids, 190 amino acids, 200 amino acids, 210 amino acids, 212 amino acids, 214 amino acids, 216 amino acids, 218 amino acids, 220 amino acids, 228 amino acids, 230 amino acids, 240 amino acids, 250 amino acids, 260 amino acids, or 270 amino acids. In particular embodiments, the spacer is 10-15 amino acids (or residues) in length. 230-240. In particular embodiments, the spacer includes a length of 229 amino acids. These lengths qualify as long spacers.

Exemplary spacers include all or a portion of an immunoglobulin hinge region. An immunoglobulin hinge region may be a wild-type immunoglobulin hinge region or an altered wild-type immunoglobulin hinge region. In certain embodiments, an immunoglobulin hinge region is a human immunoglobulin hinge region. As used herein, a “wild type immunoglobulin hinge region” refers to a naturally occurring upper and middle hinge amino acid sequences interposed between and connecting the CH1 and CH2 domains (for IgG, IgA, and IgD) or interposed between and connecting the CH1 and CH3 domains (for IgE and IgM) found in the heavy chain of an antibody.

An immunoglobulin hinge region may be an IgG, IgA, IgD, IgE, or IgM hinge region. An IgG hinge region may be an IgG1, IgG2, IgG3, or IgG4 hinge region. Sequences from IgG1, IgG2, IgG3, IgG4 or IgD can be used alone or in combination with all or a portion of a CH2 region; all or a portion of a CH3 region; or all or a portion of a CH2 region and all or a portion of a CH3 region.

In particular embodiments, the spacer is a short spacer including an IgG4 hinge region. In particular embodiments, the short spacer includes the sequence as set forth in SEQ ID NO: 15. In particular embodiments the short spacer is encoded the sequence as set forth in SEQ ID NO: 16. In particular embodiments, the spacer is a medium spacer including an IgG4 hinge region and an IgG4 CH3 region. In particular embodiments the medium spacer includes the sequence as set forth in SEQ ID NO: 13. In particular embodiments the medium spacer is encoded by SEQ ID NO: 14. In particular embodiments, the spacer is a long spacer including an IgG4 hinge region, an IgG4 CH2 region, and an IgG4 CH3 region. In particular embodiments the long spacer includes the sequence as set forth in SEQ ID NO: 11. In particular embodiments the long spacer is encoded by SEQ ID NO: 12.

Other examples of hinge regions that can be used in recombinant receptors described herein include the hinge region present in the

As used herein, a linker can include a chemical moiety that serves to connect two other subcomponents of the molecule. Some linkers serve no purpose other than to link components while many linkers serve an additional purpose. Linkers can, for example, link VL and VH of antibody derived binding domains of scFvs and serve as junction amino acids between subcomponent portions of a recombinant receptor.

Linkers can be flexible, rigid, or semi-rigid, depending on the desired function of the linker. Linkers can include junction amino acids. For example, in particular embodiments, linkers provide flexibility and room for conformational movement between different components of a recombinant receptor. Commonly used flexible linkers include Gly-Ser linkers. In particular embodiments, the linker sequence includes sets of glycine and serine repeats such as from one to ten repeats of (GlyₓSer_{y})ₙ, wherein x and y are independently an integer from 0 to 10 provided that x and y are not both 0 and wherein n is an integer of 1, 2, 3, 4, 5, 6, 7, 8, 9 or 10). Particular examples include (Gly₄Ser)ₙ (SEQ ID NO: 38), (Gly₃Ser)ₙ(Gly₄Ser)ₙ (SEQ ID NO: 39), (Gly₃Ser)ₙ(Gly₂Ser)ₙ (SEQ ID NO: 40), or (Gly₃Ser)ₙ(Gly₄Ser)₁ (SEQ ID NO: 41). In particular embodiments, the linker is (Gly₄Ser)₄ (SEQ ID NO: 42), (Gly₄Ser)₃ (SEQ ID NO: 43), (Gly₄Ser)₂ (SEQ ID NO: 44), (Gly₄Ser)₁ (SEQ ID NO: 45), (Gly₃Ser)₂ (SEQ ID NO: 46), (Gly₃Ser)₁ (SEQ ID NO: 47), (Gly₂Ser)₂ (SEQ ID NO: 48) or (Gly₂Ser)₁, GGSGGGSGGSG (SEQ ID NO: 49), GGSGGGSGSG (SEQ ID NO: 50), or GGSGGGSG (SEQ ID NO: 51).

In particular embodiments, a linker region is (GGGGS)ₙ (SEQ ID NO: 38) wherein n is an integer including, 1, 2, 3, 4, 5, 6, 7, 8, 9, or more. In particular embodiments, the spacer is (EAAAK)ₙ (SEQ ID NO: 52) wherein n is an integer including 1, 2, 3, 4, 5, 6, 7, 8, 9, or more.

In some situations, flexible linkers may be incapable of maintaining a distance or positioning of recombinant receptor needed for a particular use. In these instances, rigid or semi-rigid linkers may be useful. Examples of rigid or semi-rigid linkers include proline-rich linkers. In particular embodiments, a proline-rich linker is a peptide sequence having more proline residues than would be expected based on chance alone. In particular embodiments, a proline-rich linker is one having at least 30%, at least 35%, at least 36%, at least 39%, at least 40%, at least 48%, at least 50%, or at least 51% proline residues. Particular examples of proline-rich linkers include fragments of proline-rich salivary proteins (PRPs).

Linkers can be susceptible to cleavage (cleavable linker), such as, acid-induced cleavage, photo-induced cleavage, peptidase-induced cleavage, esterase-induced cleavage, and disulfide bond cleavage. Alternatively, linkers can be substantially resistant to cleavage (e.g., stable linker or noncleavable linker). In some aspects, the linker is a procharged linker, a hydrophilic linker, or a dicarboxylic acid-based linker.

Junction amino acids can be a linker which can be used to connect sequences when the distance provided by a spacer is not needed and/or wanted. For example, junction amino acids can be short amino acid sequences that can be used to connect co-stimulatory intracellular signaling components. In particular embodiments, junction amino acids are 9 amino acids or less (e.g., 2, 3, 4, 5, 6, 7, 8, or 9 amino acids). In particular embodiments, a glycine-serine doublet can be used as a suitable junction amino acid linker. In particular embodiments, a single amino acid, e.g., an alanine, a glycine, can be used as a suitable junction amino acid.

In particular embodiments, the recombinant receptor can optionally include a multimerization domain. Protein biological activities depend upon their tertiary and quaternary structure. The quaternary structure requires the physical and chemical interaction of different protein subunits or polypeptides. A “multimerization domain” is a domain that causes two or more proteins (monomers) to interact with each other through covalent and/or non-covalent association(s). Multimerization domains present in proteins can result in protein interactions that form dimers, trimers, tetramers, pentamers, hexamers, heptamers, etc., depending on the number of units/monomers incorporated into the multimer.

(III-B-5) Control Features Including Promoters, Tag Cassettes, Transduction Markers, Selection Cassettes, and/or Suicide Switches. In particular embodiments, genetic constructs can encode one or more control features. Control features can be used to activate, promote proliferation of, detect, enrich for, isolate, track, deplete and/or eliminate genetically modified cells in vitro, in vivo and/or ex vivo.

Promoters can include general promoters, tissue-specific promoters, cell-specific promoters, and/or promoters specific for the cytoplasm. Promoters may include strong promoters, weak promoters, constitutive expression promoters, and/or inducible promoters. Inducible promoters direct expression in response to certain conditions, signals or cellular events. For example, the promoter may be an inducible promoter that requires a particular ligand, small molecule, transcription factor or hormone protein in order to effect transcription from the promoter. Particular examples of promoters include EF-1α, CMV, Rho, SV40 immediately early promoter, the Hsp68 minimal promoter (proHSP68), and the Rous Sarcoma Virus (RSV) long-terminal repeat (LTR) promoter.

“Tag cassette” refers to a unique synthetic peptide sequence affixed to, fused to, or that is part of a recombinant receptor, to which a cognate binding molecule (e.g., ligand, antibody, or other binding partner) is capable of specifically binding where the binding property can be used to activate, promote proliferation of, detect, enrich for, isolate, track, deplete and/or eliminate the tagged protein and/or cells expressing the tagged protein.

Tag cassettes that bind cognate binding molecules include, for example, His tag (HHHHHH; SEQ ID NO: 53), Flag tag (DYKDDDDK; SEQ ID NO: 54), Xpress tag (DLYDDDDK; SEQ ID NO: 55), Avi tag (GLNDIFEAQKIEWHE; SEQ ID NO: 56), Calmodulin tag (KRRWKKNFIAVSAANRFKKISSSGAL; SEQ ID NO: 57), Polyglutamate tag, HA tag (YPYDVPDYA; SEQ ID NO: 58), Myc tag (EQKLISEEDL; SEQ ID NO: 59), Strep tag (which refers the original STREP® tag (WRHPQFGG; SEQ ID NO: 60), STREP® tag II (WSHPQFEK SEQ ID NO: 61 (IBA Institut fur Bioanalytik, Germany); see, e.g., U.S. Pat. No. 7,981,632), Softag 1 (SLAELLNAGLGGS; SEQ ID NO: 62), Softag 3 (TQDPSRVG; SEQ ID NO: 63), and V5 tag (GKPIPNPLLGLDST; SEQ ID NO: 64).

Conjugate binding molecules that specifically bind tag cassette sequences disclosed herein are commercially available. For example, His tag antibodies are commercially available from suppliers including Life Technologies, Pierce Antibodies, and GenScript. Flag tag antibodies are commercially available from suppliers including Pierce Antibodies, GenScript, and Sigma-Aldrich. Xpress tag antibodies are commercially available from suppliers including Pierce Antibodies, Life Technologies and GenScript. Avi tag antibodies are commercially available from suppliers including Pierce Antibodies, IsBio, and Genecopoeia. Calmodulin tag antibodies are commercially available from suppliers including Santa Cruz Biotechnology, Abcam, and Pierce Antibodies. HA tag antibodies are commercially available from suppliers including Pierce Antibodies, Cell Signal and Abcam. Myc tag antibodies are commercially available from suppliers including Santa Cruz Biotechnology, Abcam, and Cell Signal. Strep tag antibodies are commercially available from suppliers including Abcam, Iba, and Qiagen.

Transduction markers can serve the same purposes but are derived from naturally occurring molecules and are often expressed using a skipping element that separates the transduction marker from the rest of the recombinant receptor. Transduction markers may be selected from at least one of a truncated CD19 (tCD19; see Budde et al., Blood 122:1660, 2013); a truncated human EGFR (tEGFR or EGFRt; see Wang et al., Blood 118:1255, 2011); an ECD of human CD34; and/or RQR8 which combines target epitopes from CD34 (see Fehse et al, Mol. Therapy 1(5 Pt 1); 448-456, 2000) and CD20 antigens (see Philip et al, Blood 124: 1277-1278). Exemplary transduction markers and cognate pairs are described in U.S. Ser. No. 13/463,247. In particular embodiments, cells are genetically modified to express EGFRt.

In particular embodiments, a selection cassette provides for positive selection or negative selection of a desired cell population. Negative selection is when several cell types are removed, leaving the cell type of interest. Positive selection involves targeting the desired cell population to only retain desired cells.

A selection cassette can encode proteins that (a) confer resistance to antibiotics or other toxins, (b) complement auxotrophic deficiencies, or (c) supply critical nutrients not available from complex media, e.g., the gene encoding D-alanine racemase for Bacilli. Any number of selection systems may be used to recover transformed cells. In particular embodiments, a positive selection cassette includes resistance genes to neomycin, hygromycin, ampicillin, puromycin, phleomycin, zeomycin, blasticidin, or viomycin. In particular embodiments, a selection cassette includes the DHFR (dihydrofolate reductase) gene or DHFR double mutant (DHFRdm) gene providing resistance to methotrexate (MTX), the MGMT P140K gene responsible for the resistance to O⁶BG/BCNU, the HPRT (Hypoxanthine phosphoribosyl transferase) gene responsible for the transformation of specific bases present in the HAT selection medium (aminopterin, hypoxanthine, thymidine) or other genes for detoxification with respect to some drugs. In particular embodiments, the selection agent includes neomycin, hygromycin, puromycin, phleomycin, zeomycin, blasticidin, viomycin, ampicillin, O⁶BG/BCNU, MTX, tetracycline, aminopterin, hypoxanthine, thymidine kinase, DHFR, Gln synthetase, or ADA.

In particular embodiments, the selection cassette includes DHFRdm.

In particular embodiments, negative selection cassettes include a gene for transformation of a substrate present in the culture medium into a toxic substance for the cell that expresses the gene. These molecules include detoxification genes of diptheria toxin (DTA) (Yagi et al., Anal Biochem. 214(1):77-86, 1993; Yanagawa et al., Transgenic Res. 8(3):215-221, 1999), the kinase thymidine gene of the Herpes virus (HSV TK) sensitive to the presence of ganciclovir or FIAU. The HPRT gene may also be used as a negative selection by addition of 6-thioguanine (6TG) into the medium. and for all positive and negative selections, a poly A transcription termination sequence from different origins, the most classical being derived from SV40 poly A, or a eukaryotic gene poly A (bovine growth hormone, rabbit β-globin, etc.).

In particular embodiments, genetic constructs can include a polynucleotide that encodes a self-cleaving polypeptide, wherein the polynucleotide encoding the self-cleaving polypeptide is located between the polynucleotide encoding the recombinant receptor and a polynucleotide encoding a transduction marker (e.g., EGFRt) or selection cassette (e.g., DHFRdm). Exemplary self-cleaving polypeptides include 2A peptide from porcine teschovirus-1 (P2A), Thosea asigna virus (T2A), equine rhinitis A virus (E2A), foot-and-mouth disease virus (F2A), or variants thereof. Further exemplary nucleic acid and amino acid sequences of 2A peptides are set forth in, for example, Kim et al. (PLOS One 6:e18556 (2011). In particular embodiments, cells are genetically modified to include a self-cleaving polypeptide. In particular embodiments, the self-cleaving polypeptide includes T2A. In particular embodiments, the self-cleaving polypeptide includes P2A.

Control features may be present in multiple copies in a genetic construct or can be expressed as distinct molecules with the use of a skipping element. For example, a genetic construct can have one, two, three, four or five tag cassettes; one, two, three, four, or five transduction markers; and or one, two, three, four, or five selection cassettes could also be expressed. For example, embodiments can include a genetic construct having two Myc tag cassettes, or a His tag and an HA tag cassette, or a HA tag and a Softag 1 tag cassette, or a Myc tag and a SBP tag cassette.

One advantage of including at least one control feature in a genetic construct is that cells expressing the genetic construct administered to a subject can be increased or depleted using the cognate binding molecule to a tag cassette. In certain embodiments, the present disclosure provides a method for depleting a modified cell expressing a genetic construct by using an antibody specific for the tag cassette, using a cognate binding molecule specific for the control feature, or by using a second modified cell expressing a genetic construct and having specificity for the control feature. Elimination of modified cells may be accomplished using depletion agents specific for a control feature. For example, if EGFRt is used, then an anti-EGFRt binding domain (e.g., antibody, scFv) fused to or conjugated to a cell-toxic reagent (such as a toxin, radiometal) may be used, or an anti-EGFRt/anti-CD3 bispecific scFv, or an anti-EGFRt CAR T cell may be used.

A popular suicide switch for drug-induced cell apoptosis uses an inducible caspase suicide gene system by using a modified human caspase 9 to the FK506 binding protein (FKBP) that dimerizes in the presence of a small-molecule drug. This suicide switch is referred to as inducible caspase 9 or iCasp9 (Straathof et al., Blood. 2005, 105 (11): 4247-4254). This kill switch has shown efficacy in both preclinical and clinical contexts (Diaconu et al., Mol Ther. 2017, 25 (3): 580-592; and Stasi et al., N Engl J Med. 2011, 365 (18): 1673-1683). In particular embodiments, FDA-approved small molecules such as rapamycin can be used to control iCasp9 suicide switches (Stavrou et al., mBio. 2018, 9 (3): e00923-18).

A suicide switch can be prepared by transcriptionally linking a cell division locus (CDL) and a sequence encoding a negative selectable marker. This allows a user to inducibly kill proliferating host cells including the suicide switch or inhibit the host cell's proliferation by killing at least a portion of proliferating cells by exposing the modified cells to an inducer of the negative selectable marker. A cell modified to include the suicide switch can be treated with an inducer (e.g., a drug) of the negative selectable marker in order to ablate proliferating cells or to inhibit cell proliferation by killing at least a portion of proliferating cells.

Example CDLs include CDK1, TOP2A, CENPA, BIRC5, and EEF2. Example negative selectable markers include Herpes Simplex Virus type 1 (HSV) thymidine kinase/ganciclovir (TK/GCV), deaminase/5-fluorocytosine (CD/5-FC), and carboxyl esterase/irinotecan (CE/CPT-11).

Host cells modified with the HSV-TK/GCV negative selectable marker will produce thymidine kinase (TK) and the TK protein will convert GCV into GCV monophosphate, which is then converted into GCV triphosphate by cellular kinases. GCV triphosphate incorporates into the replicating DNA during S phase, which leads to the termination of DNA elongation and cell apoptosis (Halloran and Fenton, 1998, Cancer Res. 58(17): 3855-65).

The CD/5-FC negative selectable marker system is a widely used suicide gene system. Cytosine deaminase (CD) is a non-mammalian enzyme that may be obtained from bacteria or yeast (e.g., from Escherichia coli or Saccharomyces cerevisiae, respectively) (Ramnaraine et al., 2003). CD catalyzes conversion of cytosine into uracil and is an important member of the pyrimidine salvage pathway in prokaryotes and fungi, but it does not exist in mammalian cells. 5-fluorocytosine (5-FC) is an antifungal prodrug that causes a low level of cytotoxicity in humans (Denny, 2003, J Biomed Biotechnol). CD catalyzes conversion of 5-FC into the genotoxic agent 5-FU, which has a high level of toxicity in humans (Ireton et al., 2002, J Molec. Biol. 315(4):687-697).

The CE/CPT-11 system is based on the carboxyl esterase enzyme, which is a serine esterase found in a different tissues of mammalian species (Humerickhouse et al., 2000, Cancer Res. 60(5):1189-92). The anti-cancer agent CPT-11 is a prodrug that is activated by CE to generate an active referred to as 7-ethyl-10-hydroxycamptothecin (SN-38), which is a strong mammalian topoisomerase I inhibitor (Wierdl et al., 2001). SN-38 induces accumulation of double-strand DNA breaks in dividing cells (Kojima et al., 1998, Anal Chem. 70(13:2446-53).

In particular embodiments, the suicide switch includes the inducible caspase suicide gene system or the HSVTK/GCV suicide gene system.

In certain embodiments, modified cells expressing a genetic construct including a control feature may be detected or tracked in vivo by using antibodies that bind with specificity to a control feature (e.g., anti-Tag antibodies), or by other cognate binding molecules that specifically bind the control feature, which binding partners for the control feature are conjugated to a fluorescent dye, radio-tracer, iron-oxide nanoparticle or other imaging agent known in the art for detection by X-ray, CT-scan, MRI-scan, PET-scan, ultrasound, flow-cytometry, near infrared imaging systems, or other imaging modalities (see, e.g., Yu, et al., Theranostics 2:3, 2012).

Thus, modified cells expressing at least one control feature can be, e.g., more readily identified, isolated, sorted, induced to proliferate, tracked, and/or eliminated as compared to a modified cell without a tag cassette.

(IV) Characterization of Genetically Engineered Cells. In particular embodiments, the engineered cells can be assessed for surface expression of the recombinant receptor (e.g., CAR or eTCR). In particular embodiments, at least 50%, at least 55%, at least 60%, at least 65%, at least 70%, at least 75%, at least 80%, at least 85%, at least 90%, at least 95%, or at least 99% of the engineered cells express a detectable level of the recombinant receptor.

Surface protein expression can be determined by flow cytometry using methods known in the art. By labeling a population of cells with an element that targets the desired cell surface marker (e.g., an antibody) and is tagged with a fluorescent molecule, flow cytometry can be used to quantify the portion of the population that is positive for the surface marker, as well as the level of surface marker expression.

Genomic incorporation of a recombinant receptor within engineered cells can be determined by digital droplet PCR (ddPCR). Digital PCR enables quantification of DNA concentration in a sample. Digital PCR is performed by fractionating a mixture of a PCR reaction (e.g., containing a sample of nucleic acid molecules and copies of a PCR probe) such that some fractions contain no PCR probe copy, while other fractions contain one or more PCR probe copies. A PCR amplification of the fractions is performed and the fractions are analyzed for a PCR reaction. A fraction containing one or more probes and one or more target DNA molecules yields a positive end-point, while a fraction containing no PCR probe yields a negative end-point. The fraction of positive reactions is then fitted to a Poisson distribution to determine the absolute copy number of target DNA molecules per given volume of the unfractionated sample (i.e., copies per microliter of sample) (see Hindson, B. et al., (2011) Anal Chem. 83:8604-8610). Digital droplet PCR is a variation of digital PCR wherein a sample of nucleic acids is fractionated into droplets using a water-oil emulsion. PCR amplification is performed on the droplets collectively, whereupon a fluidics system is used to separate the droplets and provide analysis of each individual droplet. For one skilled in the art, ddPCR is used to provide an absolute quantification of DNA in a sample, to perform a copy number variation analysis, or to assess efficiency of genomic edits.

Engineered cells can also be assessed for cytokine-independent growth. Engineered cells are expected to only grow in the presence of stimulatory cytokines (e.g., IL-2, IL-7). Growth in the absence of cytokines is an indicator of tumorigenic potential. In particular embodiments, engineered cells are grown for 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, or 20 days in either the presence or in the absence of one or more stimulatory cytokines (e.g., IL-2, IL-7). In particular embodiments, proliferation is assessed by cell count and viability using conventional methods (e.g., flow cytometry, microscopy, optical density, metabolic activity). In particular embodiments, proliferation is assessed starting on day 1, day 2, day 3, day 4, day 5, day 6. In particular embodiments, proliferation is assessed every 1 day, every 2 days, every 3 days, every 4 days, every 5 days, every 6 days, every 7 days, or every 8 days. In particular embodiments, growth in the absence of cytokines is assessed at the end of a growth period. In some particular e embodiments, engineered cells with no growth in the absence of cytokines is defined as lacking tumorigenic potential. In particular embodiments, no growth is defined as an expansion of the population that is less than 0.1, 0.2, 0.3, 0.4, 0.5, 0.6, 0.7, 0.8, 0.9, 1.0, 1.1, 1.2, 1.3, 1.4, or 1.5 fold between the end of the growth period relative to the beginning of the growth period. In particular embodiments, the engineered cells do not proliferate in the absence of cytokine stimulation, growth factor stimulation, or antigen stimulation.

(V) Cell Activating Culture Conditions. Cell populations can be incubated in a culture-initiating composition to expand cell populations. The incubation can be carried out in a culture vessel, such as a bag, cell culture plate, flask, chamber, chromatography column, cross-linked gel, cross-linked polymer, column, culture dish, hollow fiber, microtiter plate, silica-coated glass plate, tube, tubing set, well, vial, or other container for culture or cultivating cells.

In particular embodiments, the cell population can be incubated in the culture-initiating composition before or after genetic engineering the cell populations. In particular embodiments, the incubation can be carried out for 1 day to 6 days, 1 day to 5 days, 1 day to 4 days, 1 day to 3 days, 1 day to 2 days, or 1 day before genetically engineering the cell populations. In particular embodiments, the incubation can be carried out for 1 day to 6 days, 1 day to 5 days, 1 day to 4 days, 1 day to 3 days, 1 day to 2 days, or 1 day after genetically engineering the cell populations. In particular embodiments, the incubation can be carried out at the same time as genetically engineering the cell populations.

Culture conditions can include one or more of particular media, temperature, oxygen content, carbon dioxide content, time, agents, e.g., nutrients, amino acids, antibiotics, ions, and/or stimulatory factors, such as cytokines, chemokines, antigens, binding partners, fusion proteins, recombinant soluble receptors, and any other agents designed to activate the cells.

In some aspects, incubation is carried out in accordance with techniques such as those described in U.S. Pat. No. 6,040,177, Klebanoff et al. (2012) J Immunother. 35(9): 651-660, Terakura et al. (2012) Blood. 1:72-82, and/or Wang et al. (2012) J Immunother. 35(9):689-701.

Exemplary culture media for culturing T cells include (i) RPMI supplemented with non-essential amino acids, sodium pyruvate, and penicillin/streptomycin; (ii) RPMI with HEPES, 5-15% human serum, 1-3% L-Glutamine, 0.5-1.5% penicillin/streptomycin, and 0.25×10-4-0.75×10-4 M β-MercaptoEthanol; (iii) RPMI-1640 supplemented with 10% fetal bovine serum (FBS), 2 mM L-glutamine, 10 mM HEPES, 100 U/ml penicillin and 100 m/mL streptomycin; (iv) DMEM medium supplemented with 10% FBS, 2 mM L-glutamine, 10 mM HEPES, 100 U/ml penicillin and 100 m/mL streptomycin; and (v) X-Vivo 15 medium (Lonza, Walkersville, MD) supplemented with 5% human AB serum (Gemcell, West Sacramento, CA), 1% HEPES (Gibco, Grand Island, NY), 1% Pen-Strep (Gibco), 1% GlutaMax (Gibco), and 2% N-acetyl cysteine (Sigma-Aldrich, St. Louis, MO). T cell culture media are also commercially available from Hyclone (Logan, UT). Additional T cell activating components that can be added to such culture media are described in more detail below.

In some embodiments, the T cells are expanded by adding to the culture-initiating composition feeder cells, such as non-dividing peripheral blood mononuclear cells (PBMC), (e.g., such that the resulting population of cells contains at least 5, 10, 20, or 40 or more PBMC feeder cells for each T lymphocyte in the initial population to be expanded); and incubating the culture (e.g., for a time sufficient to expand the numbers of T cells). In some aspects, the non-dividing feeder cells can include gamma-irradiated PBMC feeder cells. In some embodiments, the PBMC are irradiated with gamma rays in the range of 3000 to 3600 rads to prevent cell division. In some aspects, the feeder cells are added to culture medium prior to the addition of the populations of T cells.

Optionally, the incubation may further include adding non-dividing EBV-transformed lymphoblastoid cells (LCL) as feeder cells. LCL can be irradiated with gamma rays in the range of 6000 to 10,000 rads. The LCL feeder cells in some aspects is provided in any suitable amount, such as a ratio of LCL feeder cells to initial T lymphocytes of at least 10:1.

In some embodiments, the stimulating conditions include temperature suitable for the growth of human T lymphocytes, for example, at least 25° C., at least 30° C., or 37° C.

The activating culture conditions for T cells include conditions whereby T cells of the culture-initiating composition proliferate or expand. T cell activating conditions can include one or more cytokines, for example, interleukin (IL)-2, IL-7, IL-15 and/or IL-21. IL-2 can be included at a range of 10-100 ng/ml (e.g., 40, 50, or 60 ng/ml). IL-7, IL-15, and/or IL-21 can be individually included at a range of 0.1-50 ng/ml (e.g., 5, 10, or 15 ng/ml). Particular embodiments utilize IL-2 at 50 ng/ml. Particular embodiments utilize, IL-7, IL-15 and IL-21 individually included at 10 ng/ml.

In particular embodiments, T cell activating culture condition conditions can include T cell stimulating epitopes. T cell stimulating epitopes include CD3, CD27, CD2, CD4, CD5, CD7, CD8, CD28, CD30, CD40, CD56, CD83, CD90, CD95, 4-1BB (CD 137), B7-H3, CTLA-4, Frizzled-1 (FZD1), FZD2, FZD3, FZD4, FZD5, FZD6, FZD7, FZD8, FZD9, FZD10, HVEM, ICOS, IL-1R, LAT, LFA-1, LIGHT, MHCI, MHCII, NKG2D, OX40, ROR2 and RTK.

CD3 is a primary signal transduction element of T cell receptors. As indicated previously, CD3 is expressed on all mature T cells. In particular embodiments, the CD3 stimulating molecule (i.e., CD3 binding domain) can be derived from the OKT3 antibody (see U.S. Pat. Nos. 5,929,212; 4,361,549; ATCC® CRL-8001™; and Arakawa et al., J. Biochem. 120, 657-662 (1996)), the 20G6-F3 antibody, the 4B4-D7 antibody, the 4E7-C9, or the 18F5-H10 antibody.

In particular embodiments, CD3 stimulating molecules can be included within culture media at a concentration of at least 0.25 or 0.5 ng/ml or at a concentration of 2.5-10 μg/ml. Particular embodiments utilize a CD3 stimulating molecule (e.g., OKT3) at 5 μg/ml.

In particular embodiments, activating molecules associated with avi-tags can be biotinylated and bound to streptavidin beads. This approach can be used to create, for example, a removable T cell epitope stimulating activation system.

An exemplary binding domain for CD28 can include or be derived from TGN1412, CD80, CD86 or the 9D7 antibody. Additional antibodies that bind CD28 include 9.3, KOLT-2, 15E8, 248.23.2, EX5.3D10, and CD28.3 (deposited as a synthetic single chain Fv construct under GenBank Accession No. AF451974.1; see also Vanhove et al., BLOOD, 15 Jul. 2003, Vol. 102, No. 2, pages 564-570).

4-1BB binding domains can be derived from LOB12, IgG2a, LOB12.3, or IgG1 as described in Taraban et al. Eur J Immunol. 2002 December; 32(12):3617-27. In particular embodiments a 4-1BB binding domain is derived from a monoclonal antibody described in U.S. Pat. No. 9,382,328. Additional 4-1BB binding domains are described in U.S. Pat. Nos. 6,569,997, 6,303,121, and Mittler et al. Immunol Res. 2004; 29(1-3):197-208.

OX40 (CD134) and/or ICOS activation may also be used. OX40 binding domains are described in US20100196359, US20150307617, WO 2015/153513, WO2013/038191 and Melero et al. Clin Cancer Res. 2013 Mar. 1; 19(5):1044-53. Exemplary binding domains that can bind and activate ICOS are described in e.g., US20080279851 and Deng et al. Hybrid Hybridomics. 2004 June; 23(3):176-82.

When in soluble form, T cell activating agents can be coupled with another molecule, such as polyethylene glycol (PEG) molecule. Any suitable PEG molecule can be used. Typically, PEG molecules up to a molecular weight of 1000 Da are soluble in water or culture media. In some cases, such PEG based reagent can be prepared using commercially available activated PEG molecules (for example, PEG-NHS derivatives available from NOF North America Corporation, Irvine, Calif., USA, or activated PEG derivatives available from Creative PEGWorks, Chapel Hills, N.C., USA).

In particular embodiments, cell stimulating agents are immobilized on a solid phase within the culture media. In particular embodiments, the solid phase is a surface of the culture vessel (e.g., bag, cell culture plate, chamber, chromatography column, cross-linked gel, cross-linked polymer, column, culture dish, hollow fiber, microtiter plate, silica-coated glass plate, tube, tubing set, well, vial, other structure or container for culture or cultivation of cells).

In particular embodiments, a solid phase can be added to a culture media. Such solid phases can include, for example, beads, hollow fibers, resins, membranes, and polymers.

Exemplary beads include magnetic beads, polymeric beads, and resin beads (e.g., Strep-Tactin® Sepharose, Strep-Tactin® Superflow, and Strep-Tactin® MacroPrep IBA GmbH, Gottingen)). Anti-CD3/anti-CD28 beads are commercially available reagents for T cell expansion (Invitrogen). These beads are uniform, 4.5 μm superparamagnetic, sterile, non-pyrogenic polystyrene beads coated with a mixture of affinity purified monoclonal antibodies against the CD3 and CD28 cell surface molecules on human T cells. Hollow fibers are available from TerumoBCT Inc. (Lakewood, Colo., USA). Resins include metal affinity chromatography (IMAC) resins (e.g., TALON® resins (Westburg, Leusden)). Membranes include paper as well as the membrane substrate of a chromatography matrix (e.g., a nitrocellulose membrane or a polyvinylidene difluoride (PVDF) membrane).

Exemplary polymers include polysaccharides, such as polysaccharide matrices. Such matrices include agarose gels (e.g., Superflow™ agarose or a Sepharose® material such as Superflow™ Sepharose® that are commercially available in different bead and pore sizes) or a gel of crosslinked dextran(s). A further illustrative example is a particulate cross-linked agarose matrix, to which dextran is covalently bonded, that is commercially available (in various bead sizes and with various pore sizes) as Sephadex® or Superdex®, both available from GE Healthcare.

Synthetic polymers that may be used include polyacrylamide, polymethacrylate, a co-polymer of polysaccharide and agarose (e.g. a polyacrylamide/agarose composite) or a polysaccharide and N,N′-methylenebisacrylamide. An example of a copolymer of a dextran and N,N′-methylenebisacrylamide is the Sephacryl® (Pharmacia Fine Chemicals, Inc., Piscataway, NJ) series of materials.

Particular embodiments may utilize silica particles coupled to a synthetic or to a natural polymer, such as polysaccharide grafted silica, polyvinylpyrrolidone grafted silica, polyethylene oxide grafted silica, poly(2-hydroxyethylaspartamide) silica and poly(N-isopropylacrylamide) grafted silica.

Cell activating agents can be immobilized to solid phases through covalent bonds or can be reversibly immobilized through non-covalent attachments.

(VI) Ex Vivo Manufactured Cell Formulations. In particular embodiments, genetically modified cells can be harvested from a culture medium and washed and concentrated into a carrier in a therapeutically-effective amount. Exemplary carriers include saline, buffered saline, physiological saline, water, Hanks' solution, Ringer's solution, Normosol-R (Abbott Labs), PLASMA-LYTE A® (Baxter Laboratories, Inc., Morton Grove, IL), and combinations thereof.

In particular embodiments, carriers can be supplemented with human serum albumin (HSA) or other human serum components or fetal bovine serum. In particular embodiments, a carrier for infusion includes buffered saline with 5% HSA or dextrose. Additional isotonic agents include polyhydric sugar alcohols including trihydric or higher sugar alcohols, such as glycerin, erythritol, arabitol, xylitol, sorbitol, or mannitol.

Carriers can include buffering agents, such as citrate buffers, succinate buffers, tartrate buffers, fumarate buffers, gluconate buffers, oxalate buffers, lactate buffers, acetate buffers, phosphate buffers, histidine buffers, and/or trimethylamine salts.

Stabilizers refer to a broad category of excipients which can range in function from a bulking agent to an additive which helps to prevent cell adherence to container walls. Typical stabilizers can include polyhydric sugar alcohols; amino acids, such as arginine, lysine, glycine, glutamine, asparagine, histidine, alanine, ornithine, L-leucine, 2-phenylalanine, glutamic acid, and threonine; organic sugars or sugar alcohols, such as lactose, trehalose, stachyose, mannitol, sorbitol, xylitol, ribitol, myoinisitol, galactitol, glycerol, and cyclitols, such as inositol; PEG; amino acid polymers; sulfur-containing reducing agents, such as urea, glutathione, thioctic acid, sodium thioglycolate, thioglycerol, alpha-monothioglycerol, and sodium thiosulfate; low molecular weight polypeptides (i.e., <10 residues); proteins such as HSA, bovine serum albumin, gelatin or immunoglobulins; hydrophilic polymers such as polyvinylpyrrolidone; monosaccharides such as xylose, mannose, fructose and glucose; disaccharides such as lactose, maltose and sucrose; trisaccharides such as raffinose, and polysaccharides such as dextran.

Where necessary or beneficial, formulations can include a local anesthetic such as lidocaine to ease pain at a site of injection.

Exemplary preservatives include phenol, benzyl alcohol, meta-cresol, methyl paraben, propyl paraben, octadecyldimethylbenzyl ammonium chloride, benzalkonium halides, hexamethonium chloride, alkyl parabens such as methyl or propyl paraben, catechol, resorcinol, cyclohexanol, and 3-pentanol.

Therapeutically effective amounts of cells within formulations can be greater than 10² cells, greater than 10³ cells, greater than 10⁴ cells, greater than 10⁵ cells, greater than 10⁶ cells, greater than 10⁷ cells, greater than 10⁸ cells, greater than 10⁹ cells, greater than 10¹⁰ cells, or greater than 10¹¹.

In formulations disclosed herein, cells are generally in a volume of a liter or less, 500 ml or less, 250 ml or less or 100 ml or less. Hence the density of administered cells is typically greater than 10⁴ cells/ml, 10⁷ cells/ml or 10⁸ cells/ml.

In particular embodiments, formulations can include one or more genetically modified cell types (e.g., modified T cells, NK cells, or stem cells). Formulations can include different types of genetically-modified cells (e.g., T cells, NK cells, and/or stem cells in combination).

Different types of genetically-modified cells or cell subsets (e.g., modified T cells, NK cells, and/or stem cells) can be provided in different ratios e.g., a 1:1:1 ratio, 2:1:1 ratio, 1:2:1 ratio, 1:1:2 ratio, 5:1:1 ratio, 1:5:1 ratio, 1:1:5 ratio, 10:1:1 ratio, 1:10:1 ratio, 1:1:10 ratio, 2:2:1 ratio, 1:2:2 ratio, 2:1:2 ratio, 5:5:1 ratio, 1:5:5 ratio, 5:1:5 ratio, 10:10:1 ratio, 1:10:10 ratio, 10:1:10 ratio, etc. These ratios can also apply to numbers of cells expressing the same or different recombinant receptor components. If only two of the cell types are combined or only 2 combinations of expressed recombinant receptor components are included within a formulation, the ratio can include any 2-number combination that can be created from the 3 number combinations provided above. In embodiments, the combined cell populations are tested for efficacy and/or cell proliferation in vitro, in vivo and/or ex vivo, and the ratio of cells that provides for efficacy and/or proliferation of cells is selected. Particular embodiments include a 1:1 ratio of CD4 T cells and CD8 T cells.

The cell-based formulations disclosed herein can be prepared for administration by, e.g., injection, infusion, perfusion, or lavage. The formulations can further be formulated for bone marrow, intravenous, intradermal, intraarterial, intranodal, intralymphatic, intraperitoneal, intralesional, intraprostatic, intravaginal, intrarectal, intrathecal, intratumoral, intramuscular, intravesicular, and/or subcutaneous injection.

(VII) Targeted Viral Vectors & Nanoparticles for In Vivo Cell Modification. Targeted viral vectors and/or nanoparticles can also be used to genetically-modify immune cells in vivo or ex vivo. Viral vectors that can be used to deliver recombinant receptor-encoding genes to cells are described elsewhere herein, and numerous targeted (e.g., pseudotyped) viral vectors are known in the art.

Exemplary cell-targeted nanoparticles include a cell targeting ligand (e.g., CD3, CD4, CD8, CD34) on the surface of the nanoparticle wherein the cell targeting ligand results in selective uptake of the nanoparticle by a selected cell type. The nanoparticle then delivers gene modifying components that result in expression of the recombinant receptor (e.g., CAR).

Exemplary nanoparticles include liposomes (microscopic vesicles including at least one concentric lipid bilayer surrounding an aqueous core), liposomal nanoparticles (a liposome structure used to encapsulate another smaller nanoparticle within its core); and lipid nanoparticles (liposome-like structures that lack the continuous lipid bilayer characteristic of liposomes). Other polymer-based nanoparticles can also be used as well as porous nanoparticles constructed from any material capable of forming a porous network. Exemplary materials include metals, transition metals and metalloids (e.g., lithium, magnesium, zinc, aluminum and silica).

For in vivo delivery and cellular uptake, nanoparticles can have a neutral or negatively-charged coating and a size of 130 nm or less. Dimensions of the nanoparticles can be determined using, e.g., conventional techniques, such as dynamic light scattering and/or electron microscopy. In particular embodiments, the nanoparticles can be those described in WO2014153114, WO2017181110, and WO201822672.

Therapeutically effective amounts of vectors and/or nanoparticles within formulations can range from 0.1 to 5 μg/kg or from 0.5 to 1 μg/kg. In other examples, a dose can include 1 μg/kg, 30 μg/kg, 90 μg/kg, 150 μg/kg, 500 μg/kg, 750 μg/kg, 0.1 to 5 mg/kg or from 0.5 to 1 mg/kg. In other examples, a dose can include 1 mg/kg, 10 mg/kg, 30 mg/kg, 50 mg/kg, 70 mg/kg, 100 mg/kg, 300 mg/kg, 500 mg/kg, 700 mg/kg, 1000 mg/kg or more.

(viii) Methods of Use. Methods disclosed herein include treating subjects (humans, non- human primates, veterinary animals (dogs, cats, reptiles, birds, etc.) livestock (horses, cattle, goats, pigs, chickens, etc.) and research animals (monkeys, rats, mice, fish, etc.)) with formulations disclosed herein. Treating subjects includes delivering therapeutically effective amounts. Therapeutically effective amounts include those that provide effective amounts, prophylactic treatments and/or therapeutic treatments.

An “effective amount” is the amount of a formulation necessary to result in a desired physiological change in the subject. For example, an effective amount can provide an immunogenic anti-cancer effect. Effective amounts are often administered for research purposes. Effective amounts disclosed herein can cause a statistically significant effect in an animal model or in vitro assay relevant to the assessment of a cancer development or progression. An immunogenic formulation can be provided in an effective amount, wherein the effective amount stimulates an immune response.

A “prophylactic treatment” includes a treatment administered to a subject who does not display signs or symptoms of a cancer or displays only early signs or symptoms of a cancer such that treatment is administered for the purpose of diminishing or decreasing the risk of developing the cancer further. Thus, a prophylactic treatment functions as a preventative treatment against a BAFF-R-expressing cancer. In particular embodiments, prophylactic treatments reduce, delay, or prevent metastasis from a primary a cancer tumor site from occurring.

A “therapeutic treatment” includes a treatment administered to a subject who displays symptoms or signs of a cancer and is administered to the subject for the purpose of diminishing or eliminating those signs or symptoms of the cancer. The therapeutic treatment can reduce, control, or eliminate the presence or activity of the cancer and/or reduce control or eliminate side effects of the cancer.

Function as an effective amount, prophylactic treatment or therapeutic treatment are not mutually exclusive, and in particular embodiments, administered dosages may accomplish more than one treatment type.

In particular embodiments, therapeutically effective amounts provide anti-cancer effects. Anti-cancer effects include a decrease in the number of cancer cells, decrease in the number of metastases, a decrease in tumor volume, an increase in life expectancy, induced chemo- or radiosensitivity in cancer cells, inhibited cancer cell proliferation, inhibited tumor growth, prevented or reduced metastases, prolonged subject life, reduced cancer-associated pain, and/or reduced relapse or re-occurrence of cancer following treatment. In particular embodiments, therapeutically effective amounts provide anti-cancer effects in low antigen density conditions.

In particular embodiments, therapeutically effective amounts induce an immune response. The immune response can be against a BAFF-R-expressing cancer cell.

The term “BAFF-R-positive cell” refers to a cell that expresses BAFF-R on its surface. The term “BAFF-R-positive cancer cell” refers to a cancer cell that expresses BAFF-R on its surface. In some embodiments, expression of BAFF-R on the cell surface is determined, for example, using antibodies to BAFF-R in a method such as immunohistochemistry, FACS, etc. Alternatively, BAFF-R mRNA expression is considered to correlate to BAFF-R expression on the cell surface and can be determined by, for example, in situ hybridization and/or RT-PCR (including quantitative RT-PCR).

Examples of BAFF-R-related disorders that can be treated with genetic constructs disclosed herein include mantle cell lymphoma (MCL), multiple myeloma (MM), acute lymphoblastic leukemia (ALL), and diffuse large B-cell lymphoma (DLBCL).

Recombinant receptors disclosed herein can be used to treat subjects having cancers with low antigen density conditions, as described above. Certain examples include assessing a subject's cancer for BAFF-R antigen expression levels and selecting a recombinant receptor of the current disclosure to treat the subject based on the presence of low antigen density conditions.

The recombinant receptor disclosed herein are not limited to treating subjects having cancers with low antigen density conditions, and can also be used in subjects having cancers with high antigen density conditions. High antigen density conditions include those with more than 50,000 BAFF-R molecules per diseased cell; more than 60,000 BAFF-R molecules per diseased cell; more than 70,000 BAFF-R molecules per diseased cell; more than 80,000 BAFF-R molecules per diseased cell; more than 90,000 BAFF-R molecules per diseased cell; or more than 100,000 BAFF-R molecules per diseased cell.

For administration, therapeutically effective amounts (also referred to herein as doses) can be initially estimated based on results from in vitro assays and/or animal model studies. Such information can be used to more accurately determine useful doses in subjects of interest. The actual dose amount administered to a particular subject can be determined by a physician, veterinarian or researcher taking into account parameters such as physical and physiological factors including target, body weight, severity of condition, type of cancer, stage of cancer, previous or concurrent therapeutic interventions, idiopathy of the subject and route of administration.

Therapeutically effective amounts of cell-based formulations can include 10⁴ to 10⁹ cells/kg body weight, or 10³ to 10¹¹ cells/kg body weight. Therapeutically effective amounts to administer can include greater than 10² cells, greater than 10³ cells, greater than 10⁴ cells, greater than 10⁵ cells, greater than 10⁶ cells, greater than 10⁷ cells, greater than 10⁸ cells, greater than 10⁹ cells, greater than 10¹⁰ cells, or greater than 10¹¹.

Therapeutically effective amounts of vectors and/or nanoparticles within formulations can range from 0.1 to 5 μg/kg or from 0.5 to 1 μg/kg. In other examples, a dose can include 1 μg/kg, 30 μg/kg, 90 μg/kg, 150 μg/kg, 500 μg/kg, 750 μg/kg, 0.1 to 5 mg/kg or from 0.5 to 1 mg/kg. In other examples, a dose can include 1 mg/kg, 10 mg/kg, 30 mg/kg, 50 mg/kg, 70 mg/kg, 100 mg/kg, 300 mg/kg, 500 mg/kg, 700 mg/kg, 1000 mg/kg or more.

Therapeutically effective amounts can be achieved by administering single or multiple doses during the course of a treatment regimen (e.g., daily, every other day, every 3 days, every 4 days, every 5 days, every 6 days, weekly, every 2 weeks, every 3 weeks, monthly, every 2 months, every 3 months, every 4 months, every 5 months, every 6 months, every 7 months, every 8 months, every 9 months, every 10 months, every 11 months or yearly). In particular embodiments, the treatment protocol may be dictated by a clinical trial protocol or an FDA-approved treatment protocol.

Therapeutically effective amounts can be administered by, e.g., injection, infusion, perfusion, or lavage. Routes of administration can include bolus intravenous, intradermal, intraarterial, intraparenteral, intranodal, intralymphatic, intraperitoneal, intralesional, intraprostatic, intravaginal, intrarectal, topical, intrathecal, intratumoral, intramuscular, intravesicular, and/or subcutaneous administration.

In certain embodiments, formulations and/or compositions are administered to a patient in conjunction with (e.g., before, simultaneously or following) any number of relevant treatment modalities. In particular embodiments, cells may be used in combination with chemotherapy, radiation, immunosuppressive agents, such as cyclosporin, azathioprine, methotrexate, mycophenolate, and FK506, antibodies, or other immunoablative agents such as CAM PATH, anti-CD3 antibodies or other antibody therapies, cytoxin, fludaribine, cyclosporin, FK506, rapamycin, mycoplienolic acid, steroids, FR901228, cytokines, and irradiation.

In certain embodiments, cell formulations and/or modifying formulations (or formulations) may be administered in conjunction with any number of chemotherapeutic agents. Examples of chemotherapeutic agents include alkylating agents; alkyl sulfonates; aziridines; ethylenimines and methylamelamines; nitrogen mustards; nitrosureas; antibiotics; anti-metabolites; folic acid analogues; purine analogs; pyrimidine analogs; androgens; anti-adrenals; folic acid replenisher; platinum analogs; retinoic acid; and pharmaceutically acceptable salts, acids or derivatives of any of the above. Also included in this definition are anti-hormonal agents that act to regulate or inhibit hormone action on tumors such as anti-estrogens and anti-androgens and pharmaceutically acceptable salts, acids or derivatives of any of the above. Combinations of chemotherapeutic agents are also administered where appropriate, including, CHOP, i.e., Cyclophosphamide (Cytoxan®), Doxorubicin (hydroxydoxorubicin), Vincristine (Oncovin®), and Prednisone.

In some embodiments, the chemotherapeutic agent is administered at the same time or within one week after the administration of the engineered cell or nucleic acid. In other embodiments, the chemotherapeutic agent is administered from 1 to 4 weeks or from 1 week to 1 month, 1 week to 2 months, 1 week to 3 months, 1 week to 6 months, 1 week to 9 months, or 1 week to 12 months after the administration of the engineered cell or nucleic acid. In other embodiments, the chemotherapeutic agent is administered at least 1 month before administering the cell or nucleic acid. In some embodiments, the methods further include administering two or more chemotherapeutic agents.

In additional embodiments, the formulations including recombinant receptor-containing immune cells can be administered with an anti-inflammatory agent. Anti-inflammatory agents or drugs include steroids and glucocorticoids (including betamethasone, budesonide, dexamethasone, hydrocortisone acetate, hydrocortisone, hydrocortisone, methylprednisolone, prednisolone, prednisone, triamcinolone), nonsteroidal anti-inflammatory drugs (NSAIDS) including aspirin, ibuprofen, naproxen, methotrexate, sulfasalazine, leflunomide, anti-TNF medications, cyclophosphamide and mycophenolate.

In certain embodiments, the formulations and/or compositions described herein are administered in conjunction with a cytokine. “Cytokine” as used herein is meant to refer to proteins released by one cell population that act on another cell as intercellular mediators. Examples of cytokines are lymphokines, monokines, and traditional polypeptide hormones. Included among the cytokines are growth hormones such as human growth hormone, N-methionyl human growth hormone, and bovine growth hormone; parathyroid hormone; thyroxine; insulin; proinsulin; relaxin; prorelaxin; glycoprotein hormones such as follicle stimulating hormone (FSH), thyroid stimulating hormone (TSH), and luteinizing hormone (LH); hepatic growth factor (HGF); fibroblast growth factor (FGF); prolactin; placental lactogen; mullerian-inhibiting substance; mouse gonadotropin-associated peptide; inhibin; activin; vascular endothelial growth factor; integrin; thrombopoietin (TPO); nerve growth factors (NGFs) such as NGF-beta; platelet-growth factor; transforming growth factors (TGFs) such as TGF-alpha and TGF-beta; insulin-like growth factor-I and -II; erythropoietin (EPO); osteoinductive factors; interferons such as interferon-alpha, beta, and -gamma; colony stimulating factors (CSFs) such as macrophage-CSF (M-CSF); granulocyte-macrophage-CSF (GM-CSF); and granulocyte-CSF (G-CSF); interleukins (ILs) such as IL-I, IL-1 alpha, IL-2, IL-3, IL-4, IL-5, IL-6, IL-7, IL-8, IL-9, IL-10, IL-II, IL-12; IL-15, a tumor necrosis factor such as TNF-alpha or TNF-beta; and other polypeptide factors including LIF and kit ligand (KL). As used herein, the term cytokine includes proteins from natural sources or from recombinant cell culture, and biologically active equivalents of the native sequence cytokines.

In particular embodiments, an antibody or antigen binding fragment thereof that binds a transduction marker expressed by the genetically modified cell can be administered. In particular embodiments, the antibody or antigen binding fragment thereof can be a part of an antibody conjugate (i.e., an antibody linked to a detectable label or cytotoxic agent). In particular embodiments, a detectable label includes fluorescent proteins (e.g., GFP, YFP, RFP, EGFP, mCherry), radiolabels (e.g., ³⁵S, ¹²⁵I, ³²P, ³H, ¹⁴C, and ¹³¹I), radioacoustic labels, enzyme labels (e.g., horseradish peroxidase, hydrolases, luciferase, and alkaline phosphatase), chemiluminescence labels, fluorescence labels (e.g., rhodamine, phycoerythrin, and fluorescein), gold beads, magnetic beads (e.g., Dynabeads™), and biotin (with labeled avidin or streptavidin). In particular embodiments, a cytotoxic agent includes a toxin (e.g., holotoxin or hemitoxin) or a drug (e.g., chemotherapeutic agent).

In particular embodiments, genetically modified cells described herein can be further genetically modified to express a second recombinant receptor. The second recombinant receptor can provide additional cancer cell specificity by targeting cells that co-express cancer antigens or targeting cell-specific ligands. In particular embodiments, the second recombinant receptor includes a binding domain that binds a second cancer antigen, a B-cell specific ligand, or a small molecule. In particular embodiments, the second cancer antigen can include EGFR, B7H3, CD171, ROR1, HER2, IL-13Rα2, GD2, CA-125, MUC-1, EphA2, MAGE-A3, MAGE-A4, MAGE-C2, PRAME, SSX2, adipophilin, AIM2, ALDH1A1, BCLX, EpCAM, CS274, CPSF, cyclin D1, DKK1, ENAH, EPHA3, EZH2, FGF5, G250, HLA-DOB, ID01, IGF2B3, KIF20A, M-CSF, MCSP, MDM2, Meloe, MMP-2, MMP-7, MUC1, MUC5AC, p53, PAX5, PBF, PRAME, PSMA, RAGE-1, RGS5, RhoC, RNF43, RUF43, FU2AS, SOX10, STEAP1, TPBG, VEGF, WT1, or NY-ESO-1. In particular embodiments, the B-cell specific ligand includes EGFR, B7H3, CD171, ROR1, or IL-13Rα2. In particular embodiments, the B-cell specific ligand includes CD19, CD20, CD22, CD1d, CD5, CD21, CD23, CD24, CD25, CD27, CD32, CD34, CD35, CD38, CD40, CD44, CD45, CD45.1, CD45.2, CD54, CD69, CD72, CD79, CD80, CD84, LFA-1, CALLA, BCMA, B220 isoform of CD45, CD93, CD84, CD86, TNFSF7, TNFRSF5, ENPP-1, HVEM, BLIMP1, CXCR4, CD148, or CD147. In particular embodiments, the B-cell specific ligand includes CD19, CD20, or CD22. In particular embodiments, the small molecule includes fluorescein, dinitrophenol, biotin, folate, or a derivative thereof.

(VIII) Kits. The current disclosure also includes kits. Kits can include various components to practice methods disclosed herein. For example, depending on the aspect of the methods practiced, kits could include one or more of nucleic acids encoding a recombinant receptor; one or more nucleic acids encoding a CAR; nucleic acids encoding second-generation 4-1BB chimeric antigen receptors (CAR); lentiviral BAFF-R CAR construct with a short, medium, and long spacers; a nucleic acid encoding an scFv; a nucleic acid encoding a VL; a nucleic acid encoding a VH; a nucleic acid encoding a transmembrane domain; a nucleic acid encoding EGFRt; a nucleic acid encoding DHFRdm, a nucleic acid encoding Her2t, cells (e.g., immune cells, T cells, CD4 T cells, CD8 T cells, B cells, natural killer (NK) cells, NK-T cells, monocytes/macrophages, lymphocytes, hematopoietic stem cells (HSCs), hematopoietic progenitor cells (HPC), and/or a mixture of HSC and HPC (i.e., HSPC), untransduced T cells, BAFF-R-BBZ CAR T cells; cell lines (e.g., NALM6, TM-LCL, Raji, K562, lentiviral transduced cell lines); tissue samples (e.g., peripheral blood mononuclear cells (PBMCs), leukocytes, bone marrow, thymus, tissue biopsy, tumor, lymph node, gut associated lymphoid tissue, mucosa associated lymphoid tissue, spleen, other lymphoid tissues, liver, lung, stomach, intestine, colon, kidney, pancreas, breast, bone, prostate, cervix, testes, ovaries, tonsil, or other organ, and/or cells derived therefrom); genetic expression components (e.g., genes for expression provided by vectors (e.g., lentiviral vector, retroviral vector), CRISPR components, ZFNs, TALENs, MegaTALs, targeted viral vectors and/or nanoparticles); cell formulation or activation components (e.g., saline, buffered saline, phosphate buffered saline (PBS); biocompatible buffers such as, Ca++/Mg++ free PBS; physiological saline, water, Hanks' solution, Ringer's solution, T cell stimulating epitopes (e.g., anti-CD3/anti-CD28 conjugated beads; OKT3, TGN1412), culture-initiating compositions, RPMI, non-essential amino acids, sodium pyruvate, penicillin/streptomycin, non-dividing EBV-transformed lymphoblastoid cells (LCL), IL-21, human serum albumin (HSA) or other human serum components or fetal bovine serum, dextrose, stabilizers, preservatives); combination therapy components (e.g., local anesthetics, chemotherapeutic agents, immunosuppressive agents, anti-inflammatory agents); an antibody tagged with a fluorescent molecule; PCR amplification sequences; cytokines (e.g., IL-2, IL-7, IL-15, IL-21); culture vessels; GAPDH; enzyme-linked immunosorbent assay (ELISA); culture plates; etc.

The Exemplary Embodiments and Example below are included to demonstrate particular embodiments of the disclosure. Those of ordinary skill in the art should recognize in light of the present disclosure that many changes can be made to the specific embodiments disclosed herein and still obtain a like or similar result without departing from the spirit and scope of the disclosure.

### (IX) EXEMPLARY EMBODIMENTS

1. A nucleic acid including a recombinant receptor coding sequence, wherein the recombinant receptor, when expressed by a cell, includes
   an extracellular component including
      a binding domain including
         a variable heavy chain with a complementarity determining region (CDRH) 1 as set forth in SEQ ID NO: 2, a CDRH2 as set forth in SEQ ID NO: 3, and a CDRH3 as set forth in SEQ ID NO: 4, and
         a variable light chain complementarity determining region (CDRL) 1 as set forth in SEQ ID NO: 5, a CDRL2 including the sequence AAS, and a CDRL3 as set forth in SEQ ID NO: 7; and
      a spacer including an IgG4 hinge domain;
   an intracellular component including an effector domain; and
   a transmembrane domain linking the extracellular component to the intracellular component.
2. The nucleic acid of embodiment 1, wherein the intracellular component includes a CD3ζ signaling domain and a 41-BB co-stimulatory domain.
3. The nucleic acid of embodiment 2, wherein the CD34 signaling domain is encoded by the sequence as set forth in SEQ ID NO: 23.
4. The nucleic acid of embodiments 2 or 3, wherein the 41-BB co-stimulatory domain is encoded by the sequence as set forth in SEQ ID NO: 21.
5. A nucleic acid encoding a recombinant receptor, wherein the recombinant receptor, when expressed by a cell, includes
   an extracellular component including a binding domain that binds a B-cell activating factor receptor (BAFF-R);
   an intracellular component including an effector domain; and
   a transmembrane domain linking the extracellular component to the intracellular component.
6. The nucleic acid of embodiment 5, wherein the encoded recombinant receptor includes a chimeric antigen receptor (CAR), an engineered T cell receptor, or a hybrid thereof.
7. The nucleic acid of embodiments 5 or 6, wherein the encoded recombinant receptor includes a CAR.
8. The nucleic acid of any of embodiments 5-7, wherein the binding domain is derived from an H90 monoclonal antibody.
9. The nucleic acid of any of embodiments 5-8, wherein the binding domain includes: a variable heavy chain with a complementarity determining region (CDRH) 1 having at least 95% sequence identity to the sequence as set forth in SEQ ID NO: 2, a CDRH2 having at least 95% sequence identity to the sequence as set forth in SEQ ID NO: 3, and a CDRH3 having at least 95% sequence identity to the sequence as set forth in SEQ ID NO: 4 or a sequence having 0-5 conservative amino acid substitutions thereof; and
   a variable light chain complementarity determining region (CDRL) 1 having at least 95% sequence identity to the sequence as set forth in SEQ ID NO: 5, a CDRL2 having at least 95% sequence identity to the sequence AAS, and a CDRL3 having at least 95% sequence identity to the sequence as set forth in SEQ ID NO: 7 or a sequence having 0-5 conservative amino acid substitutions thereof.
10. The nucleic acid of any of embodiments 5-9, wherein the binding domain includes: a variable heavy chain with complementarity determining regions (CDRH) 1 as set forth in SEQ ID NO: 2, a CDRH2 as set forth in SEQ ID NO: 3, and a CDRH3 as set forth in SEQ ID NO: 4, and
   a variable light chain complementarity determining region (CDRL) 1 as set forth in SEQ ID NO: 5, a CDRL2 including the sequence AAS, and a CDRL3 as set forth in SEQ ID NO: 7.
11. The nucleic acid of any of embodiments 5-10, wherein the binding domain includes a variable heavy chain having at least 95% sequence identity to the sequence as set forth in SEQ ID NO: 8 or a sequence having 0-10 conservative amino acid substitutions thereof; and a variable light chain having at least 95% sequence identity to the sequence set forth in SEQ ID NO: 9 or a sequence having 0-10 conservative amino acid substitutions thereof.
12. The nucleic acid of any of embodiments 5-11, wherein the binding domain includes a variable heavy chain as set forth in SEQ ID NO: 8 and a variable light chain set forth in SEQ ID NO: 9.
13. The nucleic acid of any of embodiments 5-12, wherein the binding domain includes a humanized amino acid sequence.
14. The nucleic acid of any of embodiments 5-13, wherein the binding domain includes a humanized VH domain including a sequence having at least 95% sequence identity to the sequence as set forth in SEQ ID NO: 32, SEQ ID NO: 8, or SEQ ID NO: 34; and a humanized VL domain including a sequence having at least 95% sequence identity to the sequence as set forth in SEQ ID NO: 35, SEQ ID NO: 9, or SEQ ID NO: 37.
15. The nucleic acid of any of embodiments 5-14, wherein the binding domain includes a humanized VH domain including a sequence as set forth in SEQ ID NO: 32, SEQ ID NO: 8,or SEQ ID NO: 34; and a humanized VL domain including a sequence as set forth in SEQ ID NO: 35, SEQ ID NO: 9, or SEQ ID NO: 37.
16. The nucleic acid of any of embodiments 5-15, wherein the binding domain includes an scFv.
17. The nucleic acid of embodiment 16, wherein the scFv has at least 95% sequence identity to the sequence as set forth in SEQ ID NO: 1 or SEQ ID NO: 6.
18. The nucleic acid of embodiments 16 or 17, wherein the scFv has 1-10 conservative amino acid substitutions as compared to the sequence as set forth in SEQ ID NO: 1 or SEQ ID NO: 6.
19. The nucleic acid of any of embodiments 16-18, wherein the scFv includes the sequence as set forth in SEQ ID NO: 1 or SEQ ID NO: 6.
20. The nucleic acid of any of embodiments 5-19, wherein the extracellular component further includes a spacer.
21. The nucleic acid of embodiment 20, wherein the spacer includes a long spacer, medium spacer, or short spacer.
22. The nucleic acid of embodiments 20 or 21, wherein the spacer has a length of 10-15 residues, 110-130 residues, or 230-240 residues.
23. The nucleic acid of embodiments 21 or 22, wherein the short spacer has a length of 12 residues.
24. The nucleic acid of embodiments 21 or 22, wherein the medium spacer has a length of 119 residues.
25. The nucleic acid of embodiments 21 or 22, wherein the long spacer has a length of 229 residues.
26. The nucleic acid of any of embodiments 20-25, wherein the spacer includes an IgG4 hinge domain.
27. The nucleic acid of any of embodiments 20-26, wherein the spacer further includes an IgG4 CH3 domain.
28. The nucleic acid of any of embodiments 20-27, wherein the spacer further includes an IgG4 CH2 domain.
29. The nucleic acid of any of embodiments 20-25, wherein the spacer includes a CD8a hinge domain.
30. The nucleic acid of any of embodiments 20-29, wherein the spacer includes a sequence having at least 95% sequence identity to the sequence as set forth in SEQ ID NO: 15, SEQ ID NO: 13, SEQ ID NO: 11, or SEQ ID NO: 70, or a sequence having 0-3 conservative amino acid substitutions thereof.
31. The nucleic acid of any of embodiments 20-30, wherein the spacer includes a sequence as set forth in SEQ ID NO: 15, SEQ ID NO: 13, SEQ ID NO: 11, or SEQ ID NO: 70.
32. The nucleic acid of any of embodiments 5-31, wherein the effector domain includes all or a portion of the signaling domain of CD3ζ, CD27, CD28, 4-1BB, OX-40, CD30, CD40, PD-1, ICOS, LFA-1, CD2, CD7, NKG2C, and/or B7-H3.
33. The nucleic acid of any of embodiments 5-32, wherein the effector domain includes all or a portion of the signaling domain of CD37 and 4-1BB.
34. The nucleic acid of embodiment 33, wherein the signaling domain of CD3ζ includes a sequence having at least 95% sequence identity to the sequence as set forth in SEQ ID NO: 22.
35. The nucleic acid of embodiments 33 or 34, wherein the signaling domain of CD3ζ has a sequence having 1-5 conservative amino acid substitutions as compared to SEQ ID NO: 22.
36. The nucleic acid of embodiments 33 or 34, wherein the signaling domain of CD3Z includes a sequence as set forth in SEQ ID NO: 22.
37. The nucleic acid of any of embodiments 33-36, wherein the signaling domain of 4-1BB includes a sequence having at least 95% sequence identity to the sequence as set forth in SEQ ID NO: 20.
38. The nucleic acid of any of embodiments 33-37, wherein the signaling domain of 4-1BB has 1-5 conservative amino acid substitutions as compared to SEQ ID NO: 20.
39. The nucleic acid of any of embodiments 33-37, wherein the signaling domain of 4-1BB includes a sequence as set forth in SEQ ID NO: 20.
40. The nucleic acid of any of embodiments 5-39, wherein the transmembrane domain includes a CD28 transmembrane domain.
41. The nucleic acid of embodiment 40, wherein the CD28 transmembrane domain includes a sequence having at least 95% sequence identity to the amino acid sequence of SEQ ID NO: 17.
42. The nucleic acid of embodiments 40 or 41, wherein the CD28 transmembrane domain has 1-3 conservative amino acid substitutions as compared to SEQ ID NO: 17.
43. The nucleic acid of embodiments 40 or 41, wherein the CD28 transmembrane domain includes a sequence as set forth in SEQ ID NO: 17.
44. The nucleic acid of any of embodiments 5-43, wherein the transmembrane domain includes a CD8a transmembrane domain.
45. The nucleic acid of embodiment 44, wherein the CD8a transmembrane domain includes a sequence having at least 95% sequence identity to the amino acid sequence of SEQ ID NO: 71.
46. The nucleic acid of embodiments 44 or 45, wherein the CD8a transmembrane domain has 1-3 conservative amino acid substitutions as compared to SEQ ID NO: 71.
47. The nucleic acid of embodiments 44 or 45, wherein the CD8a transmembrane domain includes a sequence as set forth in SEQ ID NO: 71.
48. The nucleic acid of any of embodiments 5-47, further including or encoding a control feature selected from a tag cassette, a transduction marker, a selection cassette, and/or a suicide switch.
49. The nucleic acid of embodiment 48, wherein the transduction marker includes a truncated EGFR (EGFRt) polypeptide or a truncated Her2 (Her2t).
50. The nucleic acid of embodiments 48 or 49, wherein the transduction marker includes a sequence having at least 95% sequence identity to the sequence as set forth in SEQ ID NO: 29.
51. The nucleic acid of any of embodiments 48-50, wherein the transduction marker has 1-5 conservative amino acid substitutions as compared to SEQ ID NO: 29.
52. The nucleic acid of any of embodiments 48-50, wherein the transduction marker includes a sequence as set forth in SEQ ID NO: 29.
53. The nucleic acid of any of embodiments 48-52, having the transduction marker encoding sequence as set forth in SEQ ID NO: 30.
54. The nucleic acid of embodiments 48 or 49, wherein the transduction marker includes a sequence having at least 95% sequence identity to the sequence as set forth in SEQ ID NO: 85.
55. The nucleic acid of any of embodiments 48-54, wherein the transduction marker has 1-5 conservative amino acid substitutions as compared to SEQ ID NO: 85.
56. The nucleic acid of any of embodiments 48-55, wherein the transduction marker includes a sequence as set forth in SEQ ID NO: 85.
57. The nucleic acid of any of embodiments 48-56, having the transduction marker encoding sequence as set forth in SEQ ID NO: 86.
58. The nucleic acid of any of embodiments 48-57, wherein the selection cassette includes a dihydrofolate reductase double mutant (DHFRdm).
59. The nucleic acid of embodiment 58, wherein the DHFRdm includes a sequence having at least 95% sequence identity to the sequence as set forth in SEQ ID NO: 26.
60. The nucleic acid of embodiments 58 or 59, wherein the DHFRdm has 1-5 conservative amino acid substitutions as compared to SEQ ID NO: 26.
61. The nucleic acid of embodiments 58 or 59, wherein the DHFRdm includes a sequence as set forth in SEQ ID NO: 26.
62. The nucleic acid of any of embodiments 48-61, wherein the suicide switch includes herpes simplex virus thymidine kinase/ganciclovir (HSVTK/GCV) suicide gene system, or an inducible caspase suicide gene system.
63. The nucleic acid of any of embodiments 5-62, further including a ribosomal skip element.
64. The nucleic acid of embodiment 63, wherein the ribosomal skip element includes T2A, P2A, E2A, or F2A.
65. The nucleic acid of embodiments 63 or 64, wherein the ribosomal skip element includes T2A.
66. The nucleic acid of any of embodiments 63-65, wherein the ribosomal skip element includes P2A.
67. The nucleic acid sequence of any of embodiments 5-66, further including a promoter operably linked to the coding sequence encoding the recombinant receptor.
68. The nucleic acid sequence of embodiment 67, wherein the promoter includes a constitutive promoter.
69. The nucleic acid of embodiment 68, wherein the constitutive promoter includes an EF1α promoter.
70. The nucleic acid of any of embodiments 5-69, further including an inducible promoter operably linked to the coding sequence encoding the recombinant receptor.
71. The nucleic acid of any of embodiments 5-70, wherein the nucleic acid includes cDNA.
72. A nanoparticle encapsulating the nucleic acid of any of embodiments 5-71.
73. A vector including the nucleic acid any of embodiments 5-71.
74. The vector of embodiment 73, wherein the vector includes a viral vector, a transposon vector, an integrase vector, or an mRNA vector.
75. The vector of embodiment 74, wherein the viral vector includes a lentiviral vector, a foamy viral vector, a retroviral vector, or a gamma retroviral vector.
76. The vector of embodiments 74 or 75, wherein the viral vector includes a lentiviral vector.
77. Use of the nanoparticle of embodiment 72 or the vector of any of embodiments 73-76 to treat a subject in need thereof, wherein the subject has low BAFF-R antigen cell density.
78. A cell genetically modified to include the nucleic acid of any of embodiments 5-71.
79. The cell of embodiment 78, further including a genetic construct encoding a second recombinant receptor.
80. The cell of embodiment 79, wherein the second recombinant receptor includes an extracellular domain including a binding domain that binds a cancer antigen, a B-cell specific ligand, and/or a small molecule.
81. The cell of embodiment 80, wherein the cancer antigen includes EGFR, B7H3, CD171, ROR1, HER2, IL-13Rα2, GD2, CA-125, MUC-1, EphA2, MAGE-A3, MAGE-A4, MAGE-C2, PRAME, SSX2, adipophilin, AIM2, ALDH1A1, BCLX, EpCAM, CS274, CPSF, cyclin D1, DKK1, ENAH, EPHA3, EZH2, FGF5, G250, HLA-DOB, ID01, IGF2B3, KIF20A, M-CSF, MCSP, MDM2, Meloe, MMP-2, MMP-7, MUC1, MUC5AC, p53, PAX5, PBF, PRAME, PSMA, RAGE-1, RGS5, RhoC, RNF43, RUF43, FU2AS, SOX10, STEAP1, TPBG, VEGF, WT1, or NY-ESO-1.
82. The cell of embodiments 80 or 81, wherein the B-cell specific ligand includes EGFR, B7H3, CD171, ROR1, or IL-13Rα2.
83. The cell of any of embodiments 80-82, wherein the B-cell specific ligand includes CD19, CD20, CD22, CD1d, CD5, CD21, CD23, CD24, CD25, CD27, CD32, CD34, CD35, CD38, CD40, CD44, CD45, CD45.1, CD45.2, CD54, CD69, CD72, CD79, CD80, CD84, LFA-1, CALLA, BCMA, B220 isoform of CD45, CD93, CD84, CD86, TNFSF7, TNFRSF5, ENPP-1, HVEM, BLIMP1, CXCR4, CD148, or CD147.
84. The cell of any of embodiments 80-83, wherein the B-cell specific ligand includes CD19, CD20, or CD22.
85. The cell of any of embodiments 80-84, wherein the small molecule includes fluorescein, dinitrophenol, biotin, folate, or a derivative thereof.
86. The cell of any of embodiments 80-85, wherein the extracellular domain includes a spacer.
87. The cell of embodiment 86, wherein the spacer includes a long spacer, medium spacer, or short spacer.
88. The cell of embodiments 86 or 87, wherein the spacer has a length of 10-15 residues, 110-130 residues, or 230-240 residues.
89. The cell of embodiments 87 or 88, wherein the short spacer has a length of 12 residues.
90. The cell of embodiments 87 or 88, wherein the medium spacer has a length of 119 residues.
91. The cell of embodiments 87 or 88, wherein the long spacer has a length of 229 residues.
92. The cell of any of embodiments 86-91, wherein the spacer includes an IgG4 hinge domain.
93. The cell of any of embodiments 86-92, wherein the spacer further includes an IgG4 CH3 domain.
94. The cell of any of embodiments 86-93, wherein the spacer further includes an IgG4 CH2 domain.
95. The cell of any of embodiments 86-94, wherein the spacer includes a CD8a hinge domain.
96. The cell of any of embodiments 86-95, wherein the spacer includes a sequence having at least 95% sequence identity to the sequence as set forth in SEQ ID NO: 15, SEQ ID NO: 13, SEQ ID NO: 11, or SEQ ID NO: 70, or a sequence having 0-3 conservative amino acid substitutions thereof.
97. The cell of any of embodiments 86-96, wherein the spacer includes a sequence as set forth in SEQ ID NO: 15, SEQ ID NO: 13, SEQ ID NO: 11, or SEQ ID NO: 70.
98. The cell of any of embodiments 78-97, wherein the cell is an autologous cell or an allogeneic cell in reference to a subject.
99. The cell of any of embodiments 78-98, wherein the cell is a T cell, B cell, natural killer (NK) cell, NK-T cell, monocyte/macrophage, hematopoietic stem cells (HSC), or a hematopoietic progenitor cell (HPC).
100. The cell of any of embodiments 78-99, wherein the cell is a T cell selected from a CD3+ T cell, a CD4+ T cell, a CD8+ T cell, a central memory T cell, an effector memory T cell, and/or a naïve T cell.
101. The cell of any of embodiments 78-100, wherein the cell is a CD8+ T cell and/or a CD4+ T cell.
102. The cell of any of embodiments 78-102, wherein the cell is in vivo, in vitro, or ex vivo.
103. The cell of any of embodiments 78-102, wherein the cell is mammalian.
104. The cell of any of embodiments 78-103, wherein the cell is human.
105. A formulation including the cell of any of embodiments 78-104 and a pharmaceutically acceptable carrier.
106. A composition including i) the nucleic acid of any of embodiments 5-71, the nanoparticle of embodiment 72, or the vector of any of embodiments 73-76; and ii) a pharmaceutically acceptable carrier.
107. A method of treating a subject in need thereof including administering a therapeutically effective amount of the formulation of embodiment 105 and/or the composition of embodiment 106 to the subject thereby treating the subject in need thereof.
108. The method of embodiment 107, wherein the subject in need thereof has cancer.
109. The method of embodiment 108, wherein the cancer includes a BAFF-R+cell.
110. The method of embodiments 108 or 109, wherein the cancer includes a B cell cancer.
111. The method of any of embodiments 108-110, wherein cancer includes mantle cell lymphoma (MCL), multiple myeloma (MM), acute lymphoblastic leukemia (ALL), and diffuse large B-cell lymphoma (DLBCL).
112. The method of any of embodiments 107-111, wherein the formulation includes autologous cells or allogeneic cells.
113. The method of any of embodiments 107-112, wherein the subject is mammalian.
114. The method of any of embodiments 107-113, wherein the subject is human.
115. The method of any of embodiments 107-114, wherein the subject in need thereof has low BAFF-R antigen cell density.
116. The method of embodiment 115, wherein the low BAFF-R antigen cell density includes less than 50,000 BAFF-R molecules per diseased cell.
117. The method of embodiments 115 or 116, wherein the low BAFF-R antigen cell density includes less than 30,000 BAFF-R molecules per diseased cell.
118. The method of any of embodiments 115-117, wherein the low BAFF-R antigen cell density includes less than 15,000 BAFF-R molecules per diseased cell.
119. The method of any of embodiments 115-118, wherein the low BAFF-R antigen cell density includes less than 10,000 BAFF-R molecules per diseased cell.
120. The method of any of embodiments 115-119, wherein the low BAFF-R antigen cell density includes less than 7,000 BAFF-R molecules per diseased cell.
121. The method of any of embodiments 107-120, further including obtaining a sample of the diseased cells and measuring the BAFF-R antigen cell density.
122. Use of the cell of any of embodiments 78-104 in the manufacture of a medicament for treating, inhibiting or ameliorating a disorder in a subject.
123. Use of the cell of any of embodiments 78-104, to treat a subject in need thereof, wherein the subject has low BAFF-R antigen cell density.
124. The cell of any of embodiments 123 for use as a medicament.
125.A method of genetically modifying a cell including introducing the nucleic acid of any of embodiments 5-71, the nanoparticle of embodiment 72, the vector of any of embodiments 73-76, or the composition of embodiment 106 into the cell.
126. The method of embodiment 125, further including stimulating the cell by contacting the cell with a cytokine.
127. The method of embodiment 126, wherein the cytokine is selected from IL-2, IL-7, IL-15, or IL-21.
128. The method of embodiments 126 or 127, wherein the stimulating is performed prior to the introducing.
129. The method of any of embodiments 126-128, further including initiating the cell by contacting the cell with an anti-CD3 antibody or antigen binding fragment thereof, and/or an anti-CD28 antibody or antigen binding fragment thereof
130. The method of embodiment 129, wherein the initiating is performed prior to the introducing.
131. The method of any of embodiments 126-130, further including culturing the cell to obtain a plurality of the cell in an amount sufficient to administer an immunotherapy to a subject.
132. The method of any of embodiments 126-131, wherein the cell is an autologous cell or an allogeneic cell in reference to a subject.
133. The method of any of embodiments 126-132, wherein the cell is a T cell, B cell, natural killer (NK) cell, NK-T cell, monocyte/macrophage, hematopoietic stem cells (HSC), or a hematopoietic progenitor cell (HPC).
134. The method of any of embodiments 126-133, wherein the cell is a T cell selected from a CD3+ T cell, a CD4+ T cell, a CD8+ T cell, a central memory T cell, an effector memory T cell, and/or a naïve T cell.
135. The method of any of embodiments 126-134, wherein the cell is a CD8+ T cell and/or a CD4+ T cell.
136. The method of any of embodiments 126-135, wherein the cell is in vivo, in vitro, or ex vivo.
137. The method of any of embodiments 126-136, wherein the cell is mammalian.
138. The method of any of embodiments 126-137, wherein the cell is human.
139.A composition including: a recombinant receptor encoded by the nucleic acid of any of embodiments 5-71, wherein the recombinant receptor is specifically bound to a BAFF-R polypeptide or fragment thereof.
140. The composition of embodiment 139, wherein an effector cell includes the recombinant receptor.
141. The composition of embodiment 140, wherein the effector cell is a T cell, a CD4+ T cell, CD8+ T cell, a precursor T-cell, or a hematopoietic stem cell.
142. The composition of any of embodiments 139-141, wherein a target cell includes the BAFF-R polypeptide or fragment thereof.
143. The composition of embodiment 142, wherein the target cell is a B cell.
144. The composition of any of embodiments 140-143, wherein the effector cell further includes a transduction marker.
145. The composition of embodiment 144, wherein the transduction marker includes a truncated EGFR (EGFRt) polypeptide or a truncated Her2 (Her2t).
146. The composition of embodiments 144 or 145, further including an antibody or antigen binding fragment thereof specifically bound to the transduction marker.
147. The composition of embodiment 146, wherein the antibody or antigen binding fragment thereof further includes a detectable label or cytotoxic agent.

### (X) EXAMPLES

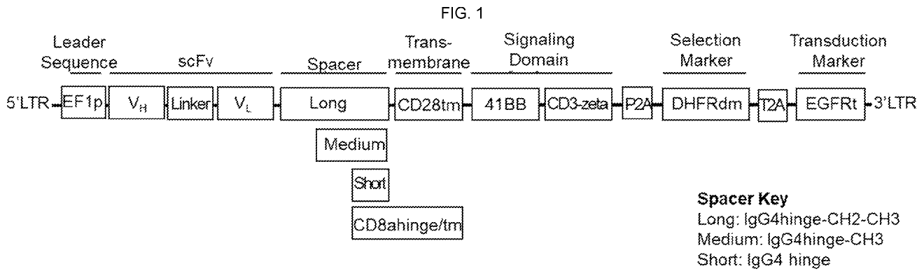

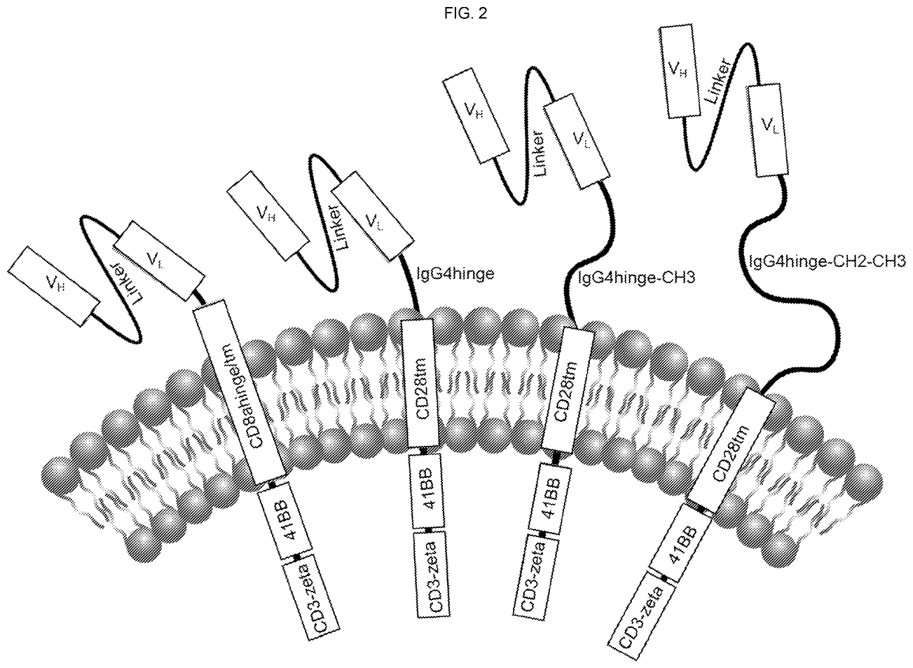

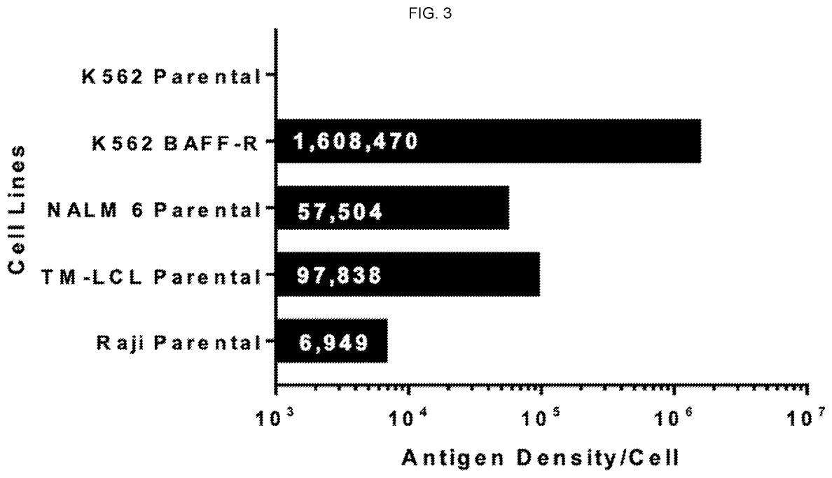

Example 1. Anti-BAFF-R CAR. Constructs depicted in FIG. 1 were prepared and levels of BAFF-R expression were measured in the following cell lines: K562, which is human immortalized myelogenous leukemia cell line; K562 BAFF-R, which included a BAFF-R expression construct; NALM6, which is a B cell precursor leukemia cell line; TM-LCL, which is a lymphoblastoid cell line; and Raji, which is a lymphoblast-like cell line. FIG. 3 depicts a bar graph of target cell antigen density for BAFF-R, and shows that endogenous expression of BAFF-R in NALM6, TM-LCL, and Raji cell lines varies, and that K562 cells transduced to express BAFF-R had substantially higher levels of expression compared to other cell lines shown.

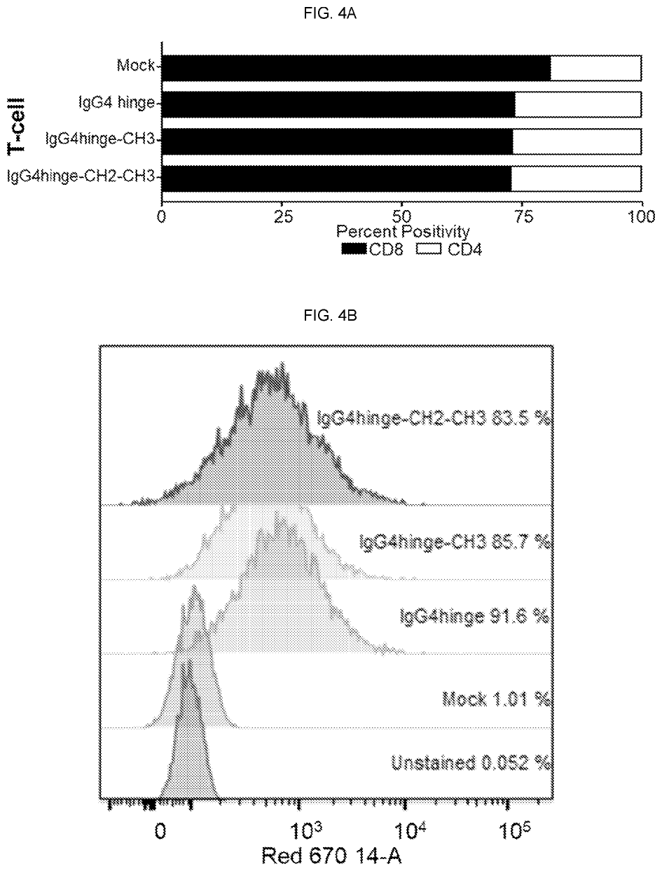

Example 2. Expression of anti-BAFF-R CAR in effector cells. Expression of anti-BAFF-R CAR in effector cells, namely CD4+ T cells and CD8+ T cells was measured. Anti-BAFF-R CAR included either an IgG4 hinge polypeptide spacer (short), an IgG4hinge-CH3 polypeptide spacer (medium), or an IgG4hinge-CH2-CH3 polypeptide spacer (long). FIGS. 4A and FIG. 4B depict proportions of CD8+ T-cells or CD4+ T cells expressing anti-BAFF-R CAR (FIG. 4A); and a fluorescence-activated cell sorting (FACS) analysis of cells expressing anti-BAFF-R CAR (FIG. 4B).

Example 3. Specific lysis of target cells by effector cells in vitro. An in vitro specific lysis assay of target cells by effector cells expressing anti-BAFF-R CAR was performed. Target cells including cell lines: K562, K562 OKT3, K562 BAFF-R, NALM6, TM-LCL, and Raji, were cultured in the presence of effector T cells expressing anti-BAFF-R CAR as set forth herein.

Lytic capability of anti-BAFF-R CAR-T cells was measured by co-culturing effector cells and fluorescent protein-expressing target cells. An Incucyte instrument was used to monitor the propagation of target cells during the co-culture period, thus providing data upon which to compare the ability of effector cells expressing each CAR to lyse a variety of target tumor cells.

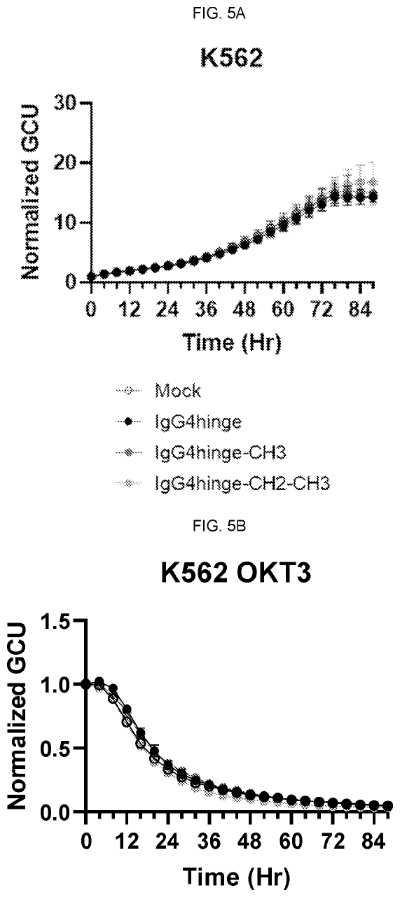

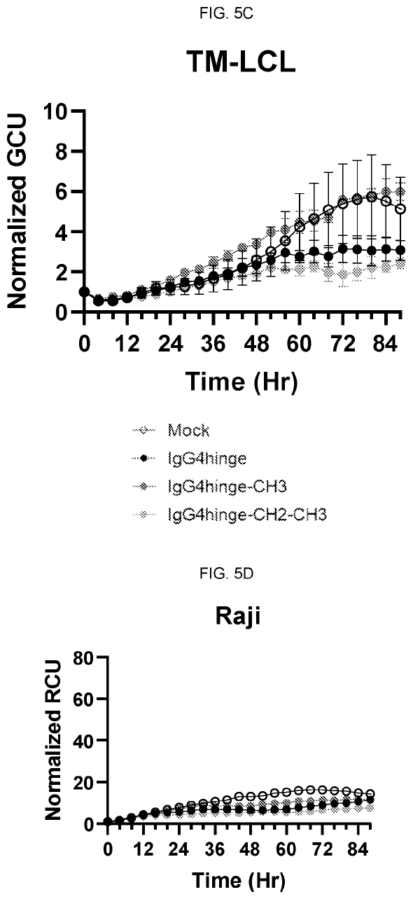

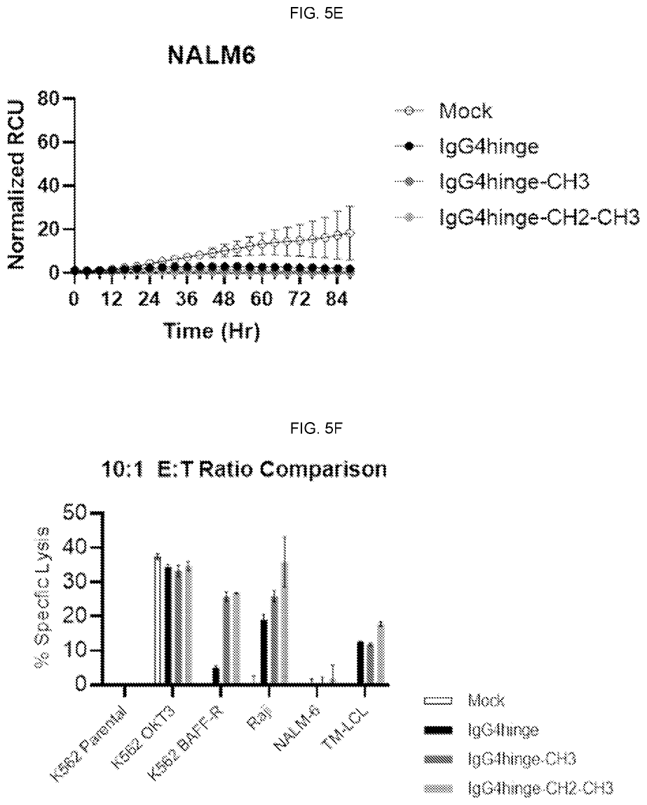

FIGS. 5A-5E depict results of a cytotoxicity assay for specific lysis of target cells by effector cells which expressed anti-BAFF-R CAR, in which target cells were K562 Parental (negative control, do not express BAFF-R), K562 OKT3+ (positive control, targeted for killing by T cell receptor), and BAFF-R+ targets K562 BAFF-R+, TM-LCL, Raji, and NALM6, respectively. FIG. 5F summarizes the results and provides a bar graph for specific percentage lysis of target cells by effector cells expressing anti-BAFF-R CAR.

Example 4. Cytokine induction in target cells by effector cells in vitro. An in vitro cytokine induction assay of target cells by effector cells expressing anti-BAFF-R CAR was performed. Target cells including cell lines: K562, K562 OKT3, K562 BAFF-R, NALM6, TM-LCL, and Raji, were cultured in the presence of effector T cells expressing anti-BAFF-R CAR as set forth herein.

The ability of each CAR to induce the production of cytokines in the presence of target cells was measured using a co-culture assay. Briefly, effector cells and target cells were combined in the well of a microplate and allowed to incubate for 24 hours. After the incubation time, supernatants from each co-culture were analyzed for interferon gamma (IFNγ), interleukin-2 (IL-2), and tumor necrosis factor alpha (TNFα).

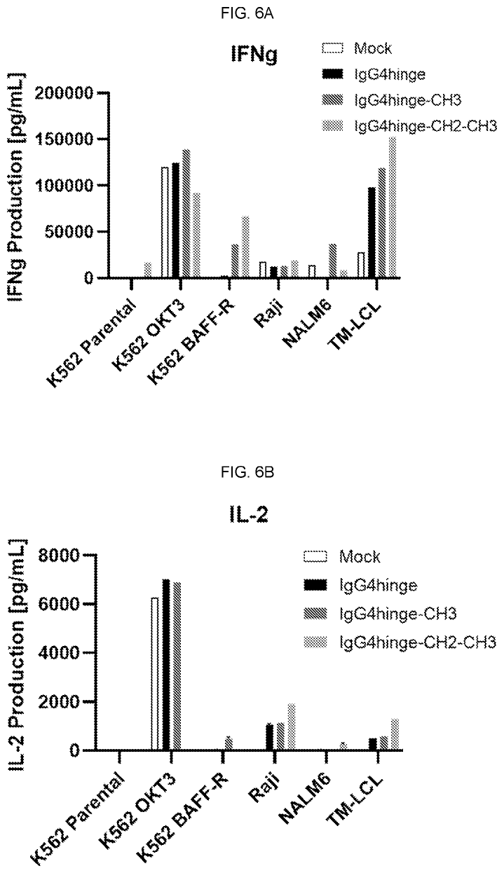

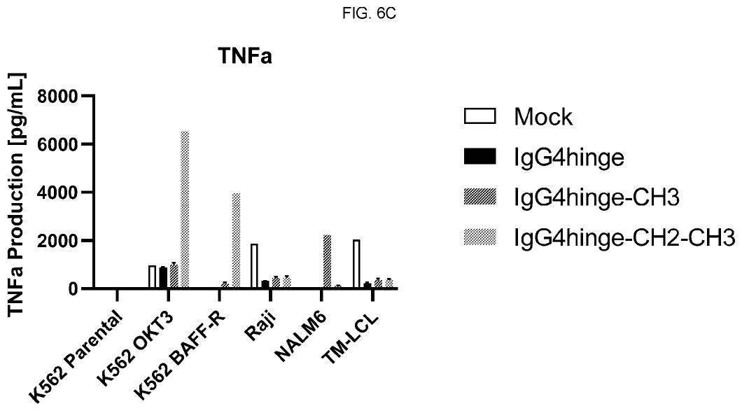

Results are shown in FIGS. 6A-6C, which depict bar graphs for cytokine production induced in effector cells expressing anti-BAFF-R CAR in the presence of target cells K562 Parental (negative control, do not express BAFF-R), K562 OKT3+ (positive control, targeted for killing by T cell receptor), and BAFF-R+ targets K562 BAFF-R+, TM-LCL, Raji, and NALM6, for interferon gamma (IFNγ), interleukin-2 (IL-2), and tumor necrosis factor alpha (TNFα), respectively.

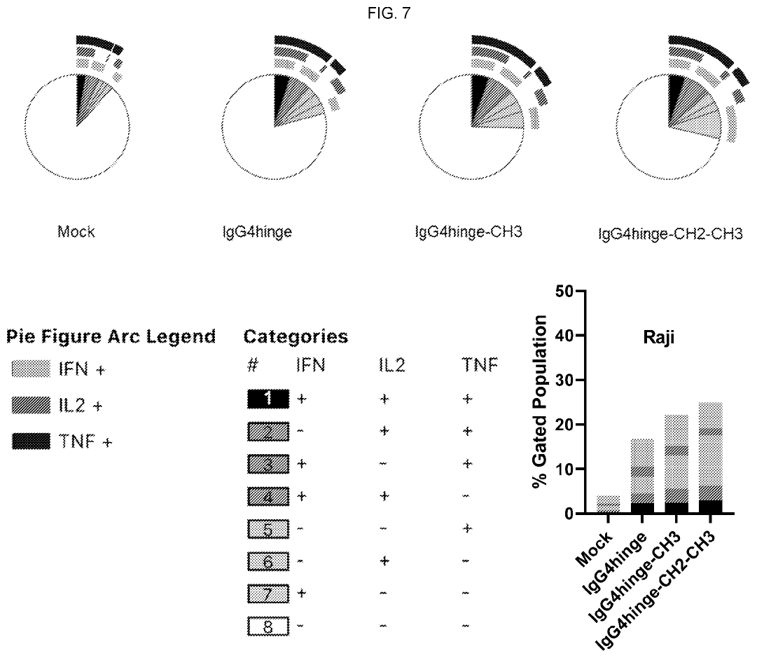

FIG. 7 depicts the proportion of gated cells (gated on: lymphocytes/single/live/CD8/EGFRt+) in a series of pie charts (upper panel), and a bar graph (lower right panel) for expression of cytokines (IFNγ, IL-2, TNFα) induced in CD8+ T cells expressing an anti-BAFF-R CAR in the presence of target Raji cells.

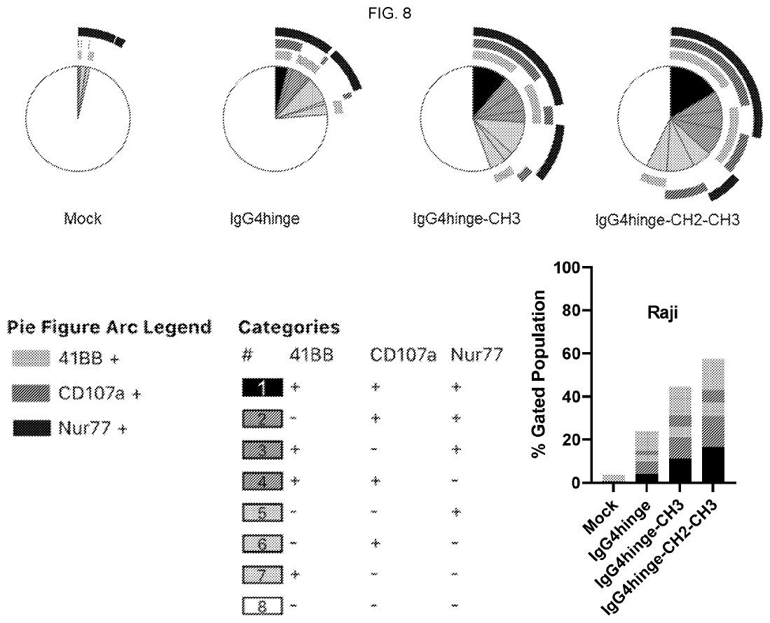

FIG. 8 depicts the proportion of gated cells (gated on: lymphocytes/single/live/CD8/EGFRt+) in a series of pie charts (upper panel), and a bar graph (lower right panel) for expression of 4-1BB, CD107a, or Nur77 induced in CD8+ T cells expressing an anti-BAFF-R CAR in the presence of target Raji cells.

Example 5. In vivo administration of effector cells in a xenomorphic model. Activity of anti-BAFF-R CAR T cells was investigated in a xenomorphic model. Mice were administered Raji cells expressing reporter genes: mCherry and luciferase, and treated with T cells expressing anti-BAFF-R CAR. Mice were imaged over the course of the study by intraperitoneal (i.p.) injection of D-luciferin followed by imaging using an IVIS Spectrum imager, which detected luminescent tumor cells in the mice. During the course of the study, mice were evaluated for signs of increased tumor burden, xenogeneic graft-versus-host disease (xGVHD), and toxicity. On two occasions, days 35 and 60, mice were re-administered a dose of Raji cells to determine the durability of T cell engraftment and to evaluate the ability of T cells with each CAR to resist exhaustion.

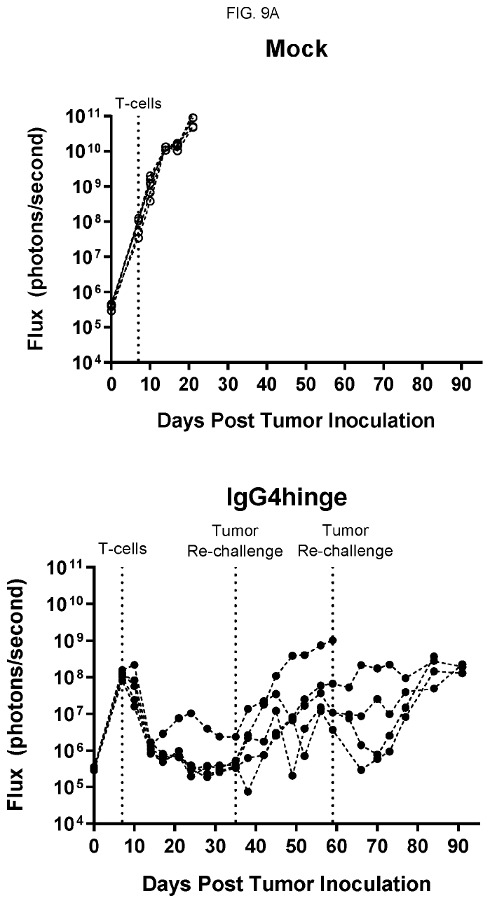

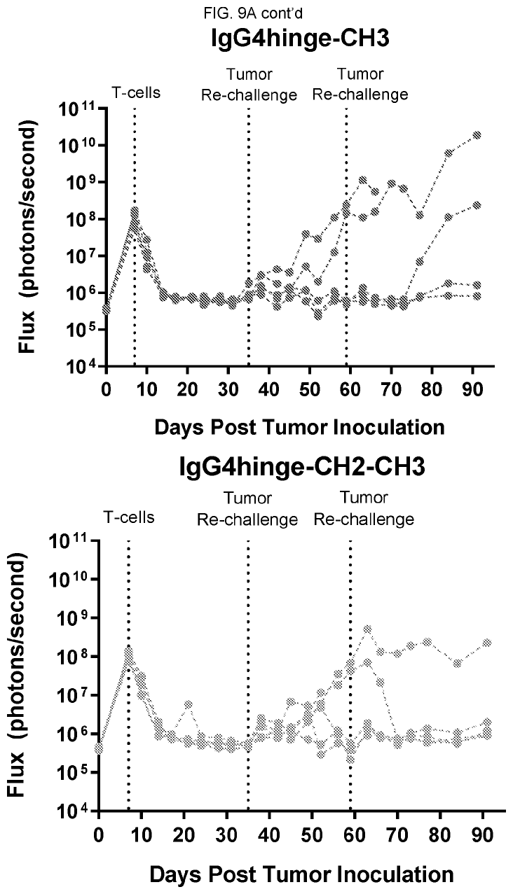

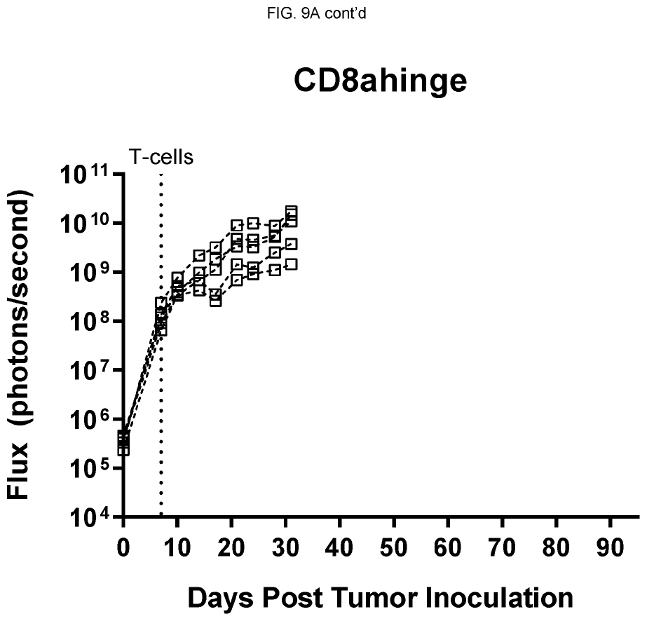

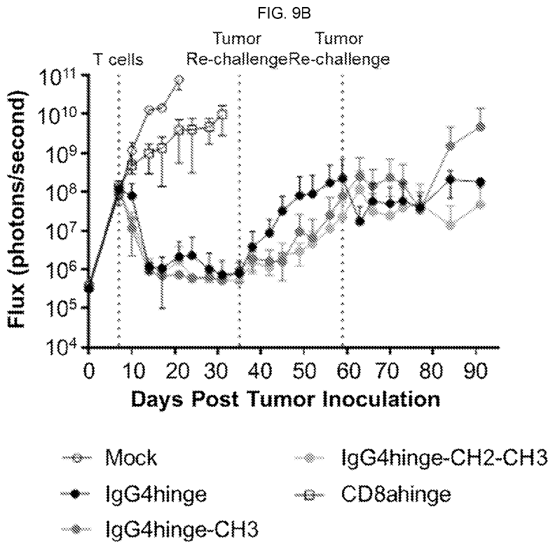

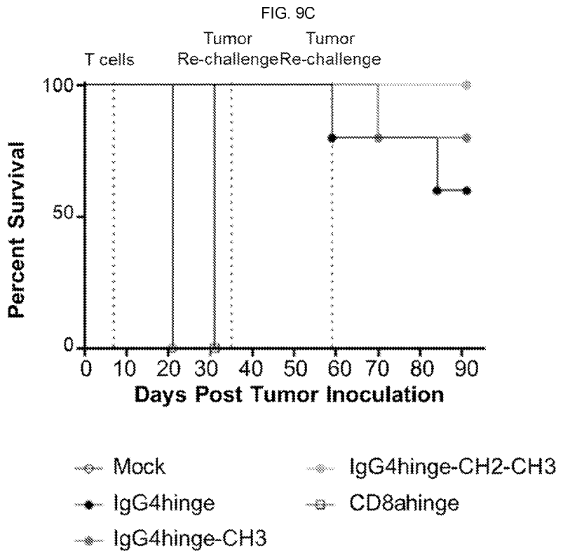

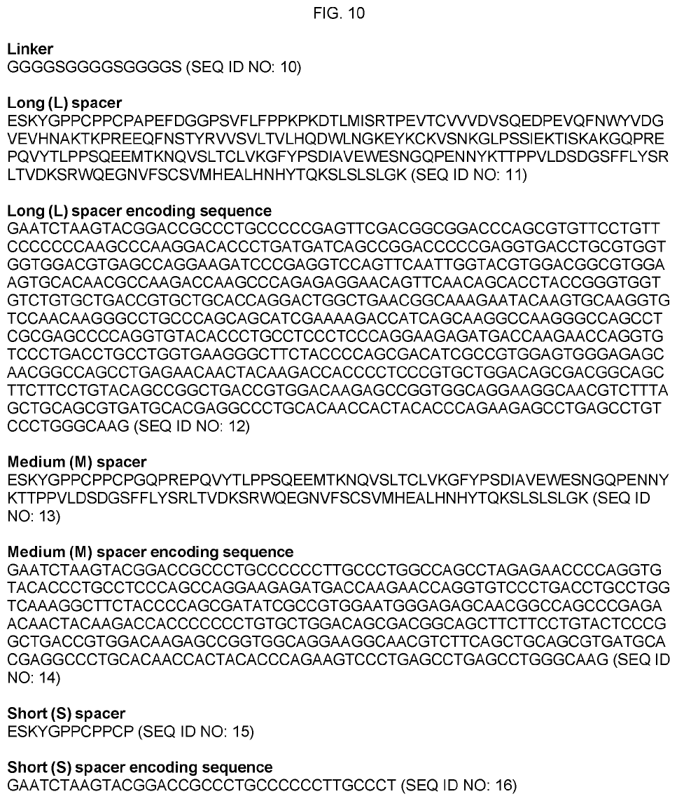

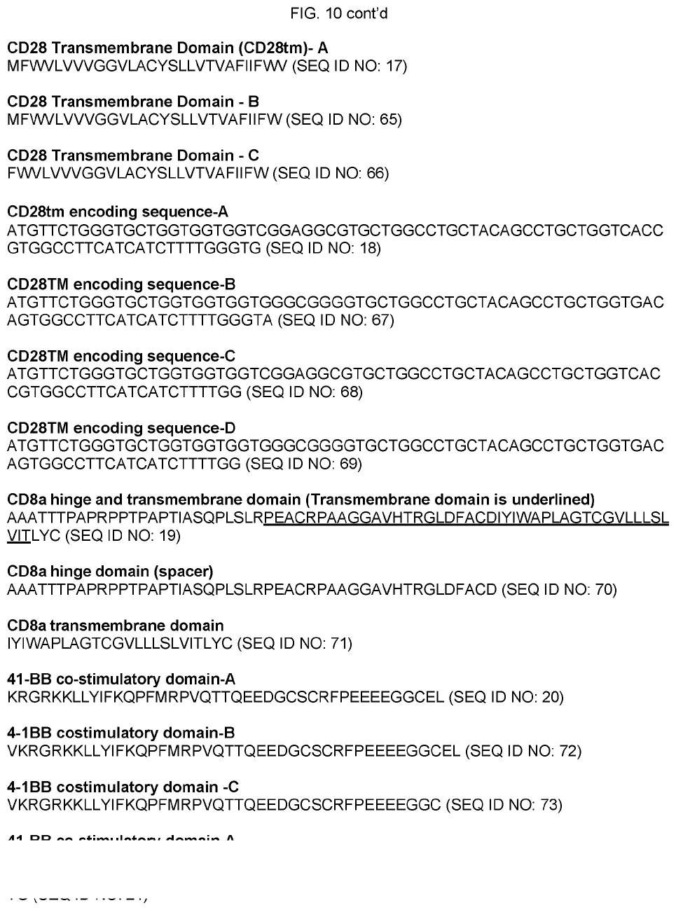

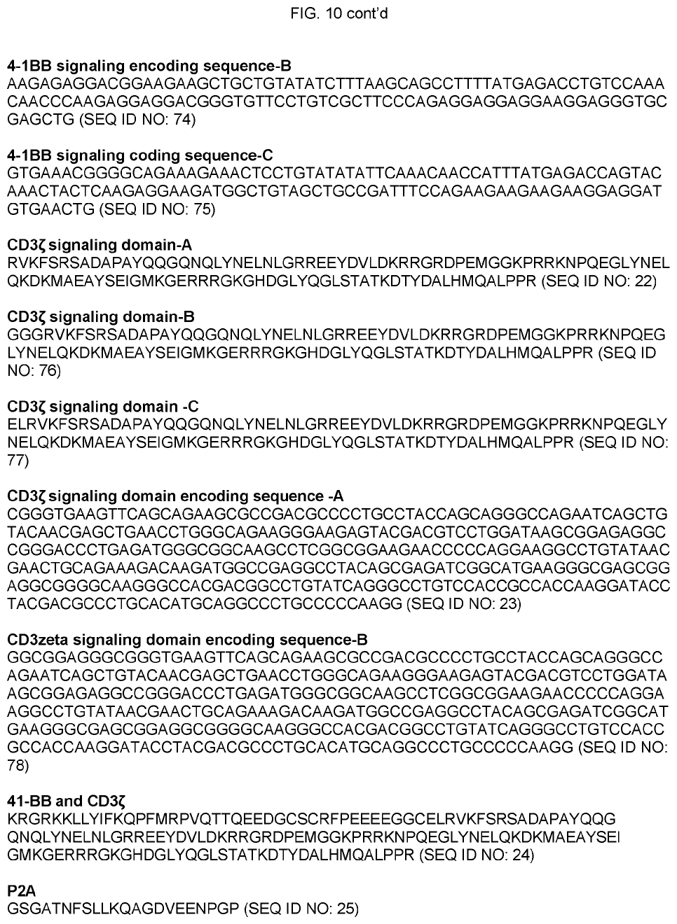

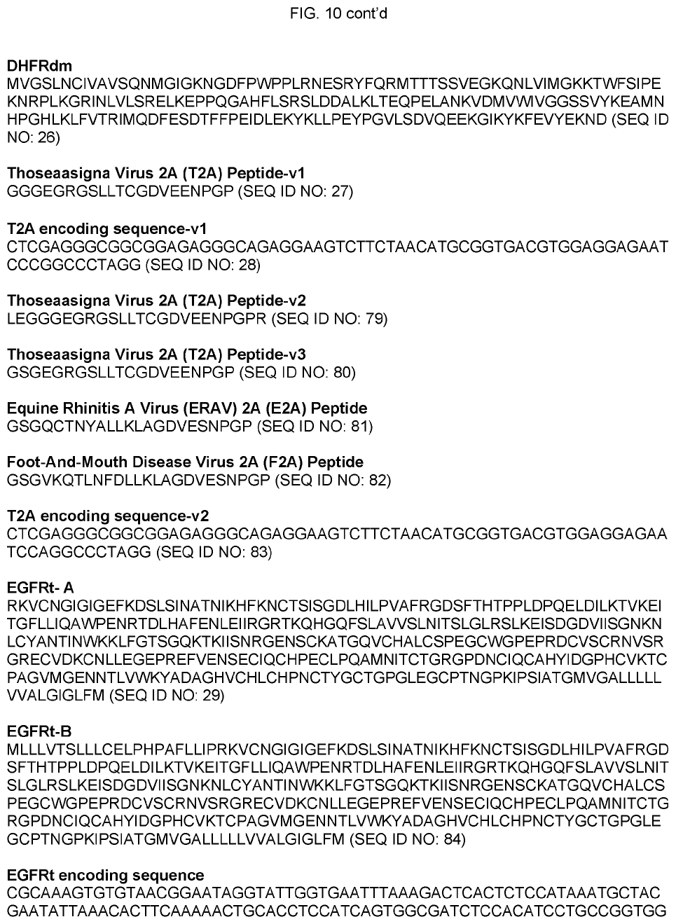

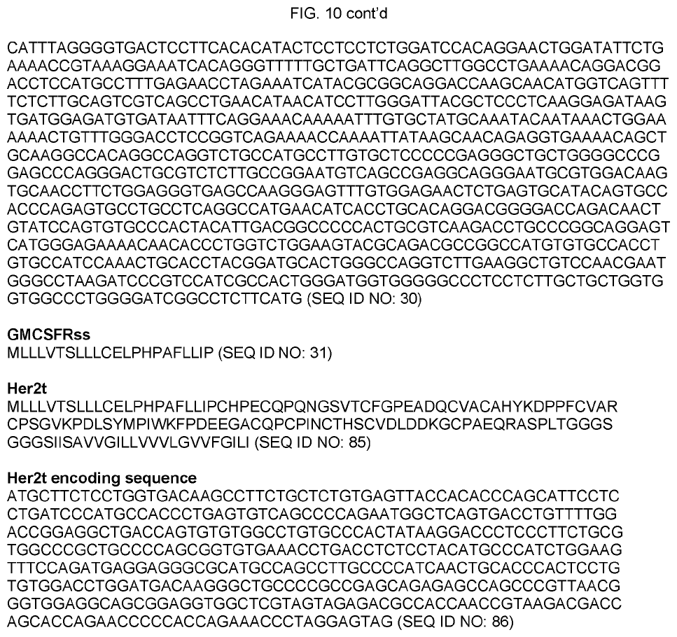

FIG. 9A depicts a series of graphs of bioluminescence in mice, with each line indicative of a subject. FIG. 9B depicts a graph of average bioluminescence for the results shown in FIG. 9A. FIG. 9C depicts percentage survival and tumor related death for mice treated as described for FIG. 9A. As shown in FIG. 9A and FIG. 9B, unexpectedly, mice contacted with anti-BAFF-R CAR T cells in which the CAR contained an IgG4 hinge polypeptide spacer had (i) a lower tumor burden, as indicated by lower flux levels, and (ii) longer survival, as compared to mice treated with anti-BAFF-R CAR T cells in which the CAR contained a CD8a hinge polypeptide spacer.

(XI) Closing Paragraphs. The nucleic acid and amino acid sequences provided herein are shown using letter abbreviations for nucleotide bases and amino acid residues, as defined in 37 C.F.R. §1.831-1.835 and set forth in WIPO Standard ST.26 (implemented on Jul. 1, 2022). Only one strand of each nucleic acid sequence is shown, but the complementary strand is understood as included in embodiments where it would be appropriate.

Variants of the sequences disclosed and referenced herein are also included. Guidance in determining which amino acid residues can be substituted, inserted, or deleted without abolishing biological activity can be found using computer programs well known in the art, such as DNASTAR™ (Madison, Wisconsin) software. Preferably, amino acid changes in the protein variants disclosed herein are conservative amino acid changes, i.e., substitutions of similarly charged or uncharged amino acids. A conservative amino acid change involves substitution of one of a family of amino acids which are related in their side chains.

In a peptide or protein, suitable conservative substitutions of amino acids are known to those of skill in this art and generally can be made without altering a biological activity of a resulting molecule. Those of skill in this art recognize that, in general, single amino acid substitutions in non-essential regions of a polypeptide do not substantially alter biological activity (see, e.g., Watson et al. Molecular Biology of the Gene, 4th Edition, 1987, The Benjamin/Cummings Pub. Co., p. 224). Naturally occurring amino acids are generally divided into conservative substitution families as follows: Group 1: Alanine (Ala), Glycine (Gly), Serine (Ser), and Threonine (Thr); Group 2: (acidic): Aspartic acid (Asp), and Glutamic acid (Glu); Group 3: (acidic; also classified as polar, negatively charged residues and their amides): Asparagine (Asn), Glutamine (Gln), Asp, and Glu; Group 4: Gln and Asn; Group 5: (basic; also classified as polar, positively charged residues): Arginine (Arg), Lysine (Lys), and Histidine (His); Group 6 (large aliphatic, nonpolar residues): Isoleucine (Ile), Leucine (Leu), Methionine (Met), Valine (Val) and Cysteine (Cys); Group 7 (uncharged polar): Tyrosine (Tyr), Gly, Asn, Gln, Cys, Ser, and Thr; Group 8 (large aromatic residues): Phenylalanine (Phe), Tryptophan (Trp), and Tyr; Group 9 (non-polar): Proline (Pro), Ala, Val, Leu, Ile, Phe, Met, and Trp; Group 11 (aliphatic): Gly, Ala, Val, Leu, and Ile; Group 10 (small aliphatic, nonpolar or slightly polar residues): Ala, Ser, Thr, Pro, and Gly; and Group 12 (sulfur-containing): Met and Cys. Additional information can be found in Creighton (1984) Proteins, W.H. Freeman and Company.

In making such changes, the hydropathic index of amino acids may be considered. The importance of the hydropathic amino acid index in conferring interactive biologic function on a protein is generally understood in the art (Kyte and Doolittle, 1982, J. Mol. Biol. 157(1), 105-32). Each amino acid has been assigned a hydropathic index on the basis of its hydrophobicity and charge characteristics (Kyte and Doolittle, 1982). These values are: Ile (+4.5); Val (+4.2); Leu (+3.8); Phe (+2.8); Cys (+2.5); Met (+1.9); Ala (+1.8); Gly (−0.4); Thr (−0.7); Ser (−0.8); Trp (−0.9); Tyr (−1.3); Pro (−1.6); His (−3.2); Glutamate (−3.5); Gln (−3.5); aspartate (−3.5); Asn (−3.5); Lys (−3.9); and Arg (−4.5).

It is known in the art that certain amino acids may be substituted by other amino acids having a similar hydropathic index or score and still result in a protein with similar biological activity, i.e., still obtain a biological functionally equivalent protein. In making such changes, the substitution of amino acids whose hydropathic indices are within ±2 is preferred, those within ±1 are particularly preferred, and those within ±0.5 are even more particularly preferred. It is also understood in the art that the substitution of like amino acids can be made effectively on the basis of hydrophilicity.

As detailed in U.S. Pat. No. 4,554,101, the following hydrophilicity values have been assigned to amino acid residues: Arg (+3.0); Lys (+3.0); aspartate (+3.0+1); glutamate (+3.0+1); Ser (+0.3); Asn (+0.2); Gln (+0.2); Gly (0); Thr (−0.4); Pro (−0.5±1); Ala (−0.5); His (−0.5); Cys (−1.0); Met (−1.3); Val (−1.5); Leu (−1.8); Ile (−1.8); Tyr (−2.3); Phe (−2.5); Trp (−3.4). It is understood that an amino acid can be substituted for another having a similar hydrophilicity value and still obtain a biologically equivalent, and in particular, an immunologically equivalent protein. In such changes, the substitution of amino acids whose hydrophilicity values are within ±2 is preferred, those within ±1 are particularly preferred, and those within ±0.5 are even more particularly preferred.

As outlined above, amino acid substitutions may be based on the relative similarity of the amino acid side-chain substituents, for example, their hydrophobicity, hydrophilicity, charge, size, and the like. As indicated elsewhere, variants of gene sequences can include codon optimized variants, sequence polymorphisms, splice variants, and/or mutations that do not affect the function of an encoded product to a statistically-significant degree.

Variants of the protein, nucleic acid, and gene sequences disclosed herein also include sequences with at least 70% sequence identity, 80% sequence identity, 85% sequence, 90% sequence identity, 95% sequence identity, 96% sequence identity, 97% sequence identity, 98% sequence identity, or 99% sequence identity to the protein, nucleic acid, or gene sequences disclosed herein.

“% sequence identity” refers to a relationship between two or more sequences, as determined by comparing the sequences. In the art, “identity” also means the degree of sequence relatedness between protein, nucleic acid, or gene sequences as determined by the match between strings of such sequences. “Identity” (often referred to as “similarity”) can be readily calculated by known methods, including those described in: Computational Molecular Biology (Lesk, A. M., ed.) Oxford University Press, NY (1988); Biocomputing: Informatics and Genome Projects (Smith, D. W., ed.) Academic Press, NY (1994); Computer Analysis of Sequence Data, Part I (Griffin, A. M., and Griffin, H. G., eds.) Humana Press, NJ (1994); Sequence Analysis in Molecular Biology (Von Heijne, G., ed.) Academic Press (1987); and Sequence Analysis Primer (Gribskov, M. and Devereux, J., eds.) Oxford University Press, NY (1992). Methods to determine identity are designed to give the best match between the sequences tested. Methods to determine identity and similarity are codified in publicly available computer programs. Sequence alignments and percent identity calculations may be performed using the Megalign program of the LASERGENE bioinformatics computing suite (DNASTAR, Inc., Madison, Wisconsin). Multiple alignment of the sequences can also be performed using the Clustal method of alignment (Higgins and Sharp CABIOS, 5, 151-153 (1989) with default parameters (GAP PENALTY=10, GAP LENGTH PENALTY=10). Relevant programs also include the GCG suite of programs (Wisconsin Package Version 9.0, Genetics Computer Group (GCG), Madison, Wisconsin); BLASTP, BLASTN, BLASTX (Altschul, et al., J. Mol. Biol. 215:403-410 (1990); DNASTAR (DNASTAR, Inc., Madison, Wisconsin); and the FASTA program incorporating the Smith-Waterman algorithm (Pearson, Comput. Methods Genome Res., [Proc. Int. Symp.] (1994), Meeting Date 1992, 111-20. Editor(s): Suhai, Sandor. Publisher: Plenum, New York, N.Y. Within the context of this disclosure it will be understood that where sequence analysis software is used for analysis, the results of the analysis are based on the “default values” of the program referenced. As used herein “default values” will mean any set of values or parameters, which originally load with the software when first initialized.

Variants also include nucleic acid molecules that hybridize under stringent hybridization conditions to a sequence disclosed herein and provide the same function as the reference sequence. Exemplary stringent hybridization conditions include an overnight incubation at 42° C. in a solution including 50% formamide, 5×SSC (750 mM NaCl, 75 mM trisodium citrate), 50 mM sodium phosphate (pH 7.6), 5×Denhardt′s solution, 10% dextran sulfate, and 20 μg/ml denatured, sheared salmon sperm DNA, followed by washing the filters in 0.1×SSC at 50° C. Changes in the stringency of hybridization and signal detection are primarily accomplished through the manipulation of formamide concentration (lower percentages of formamide result in lowered stringency); salt conditions, or temperature. For example, moderately high stringency conditions include an overnight incubation at 37° C. in a solution including 6×SSPE (20×SSPE=3M NaCl; 0.2M NaH2PO4; 0.02M EDTA, pH 7.4), 0.5% SDS, 30% formamide, 100 μg/ml salmon sperm blocking DNA; followed by washes at 50° C. with 1×SSPE, 0.1% SDS. In addition, to achieve even lower stringency, washes performed following stringent hybridization can be done at higher salt concentrations (e.g. 5×SSC). Variations in the above conditions may be accomplished through the inclusion and/or substitution of alternate blocking reagents used to suppress background in hybridization experiments. Typical blocking reagents include Denhardt's reagent, BLOTTO, heparin, denatured salmon sperm DNA, and commercially available proprietary formulations. The inclusion of specific blocking reagents may require modification of the hybridization conditions described above, due to problems with compatibility.

“Binds” refers to an association of a binding domain (of, for example, a recombinant receptor binding domain) to its cognate binding molecule with an affinity or Kₐ (i.e., an equilibrium association constant of a particular binding interaction with units of 1/M) equal to or greater than 10⁵ M⁻¹, while not significantly associating with any other molecules or components in a relevant environment sample. Binding domains may be classified as “high affinity” or “low affinity”. In particular embodiments, “high affinity” binding domains refer to those binding domains with a Kₐ of at least 10⁷ M⁻¹, at least 10⁸ M⁻¹, at least 10⁹ M⁻¹, at least 10¹⁰ M⁻¹, at least 10¹¹ M⁻¹, at least 10¹² M⁻¹, or at least 10¹³ M⁻¹. In particular embodiments, “low affinity” binding domains refer to those binding domains with a Kₐ of up to 10⁷ M⁻¹, up to 10⁶ M⁻¹, up to 10⁵ M⁻¹. Alternatively, affinity may be defined as an equilibrium dissociation constant (K_{d}) of a particular binding interaction with units of M (e.g., 10⁻⁵ M to 10⁻¹³ M). In certain embodiments, a binding domain may have “enhanced affinity,” which refers to a selected or engineered binding domains with stronger binding to a cognate binding molecule than a wild type (or parent) binding domain. For example, enhanced affinity may be due to a Kₐ (equilibrium association constant) for the cognate binding molecule that is higher than the reference binding domain or due to a K_{d} (dissociation constant) for the cognate binding molecule that is less than that of the reference binding domain, or due to an off-rate (K_{off}) for the cognate binding molecule that is less than that of the reference binding domain. A variety of assays are known for detecting binding domains that specifically bind a particular cognate binding molecule as well as determining binding affinities, such as Western blot, ELISA, and BIACORE® analysis (see also, e.g., Scatchard, et al., 1949, Ann. N. Y. Acad. Sci. 51:660; and U.S. Pat. Nos. 5,283,173, 5,468,614, or the equivalent).

Unless otherwise indicated, the practice of the present disclosure can employ conventional techniques of immunology, molecular biology, microbiology, cell biology and recombinant DNA. These methods are described in the following publications. See, e.g., Sambrook, et al. Molecular Cloning: A Laboratory Manual, 2nd Edition (1989); F. M. Ausubel, et al. eds., Current Protocols in Molecular Biology, (1987); the series Methods IN Enzymology (Academic Press, Inc.); M. MacPherson, et al., PCR: A Practical Approach, IRL Press at Oxford University Press (1991); MacPherson et al., eds. PCR 2: Practical Approach, (1995); Harlow and Lane, eds. Antibodies, A Laboratory Manual, (1988); and R. I. Freshney, ed. Animal Cell Culture (1987).

As will be understood by one of ordinary skill in the art, each embodiment disclosed herein can comprise, consist essentially of or consist of its particular stated element, step, ingredient or component. Thus, the terms “include” or “including” should be interpreted to recite: “comprise, consist of, or consist essentially of.” The transition term “comprise” or “comprises” means has, but is not limited to, and allows for the inclusion of unspecified elements, steps, ingredients, or components, even in major amounts. The transitional phrase “consisting of” excludes any element, step, ingredient or component not specified. The transition phrase “consisting essentially of” limits the scope of the embodiment to the specified elements, steps, ingredients or components and to those that do not materially affect the embodiment. A material effect would cause a statistically significant increase in BAFF-R-expressing cells in a treated subject according to experimental protocols described herein.

Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques. When further clarity is required, the term “about” has the meaning reasonably ascribed to it by a person skilled in the art when used in conjunction with a stated numerical value or range, i.e. denoting somewhat more or somewhat less than the stated value or range, to within a range of ±20% of the stated value; ±19% of the stated value; ±18% of the stated value; ±17% of the stated value; ±16% of the stated value; ±15% of the stated value; ±14% of the stated value; ±13% of the stated value; ±12% of the stated value; ±11% of the stated value; ±10% of the stated value; ±9% of the stated value; ±8% of the stated value; ±7% of the stated value; ±6% of the stated value; ±5% of the stated value; ±4% of the stated value; ±3% of the stated value; ±2% of the stated value; or ±1% of the stated value.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

The terms “a,” “an,” “the” and similar referents used in the context of describing the invention (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Certain embodiments of this invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Furthermore, numerous references have been made to patents, printed publications, journal articles and other written text throughout this specification (referenced materials herein). Each of the referenced materials are individually incorporated herein by reference in their entirety for their referenced teaching.

In closing, it is to be understood that the embodiments of the invention disclosed herein are illustrative of the principles of the present invention. Other modifications that may be employed are within the scope of the invention. Thus, by way of example, but not of limitation, alternative configurations of the present invention may be utilized in accordance with the teachings herein. Accordingly, the present invention is not limited to that precisely as shown and described.

The particulars shown herein are by way of example and for purposes of illustrative discussion of the preferred embodiments of the present invention only and are presented in the cause of providing what is believed to be the most useful and readily understood description of the principles and conceptual aspects of various embodiments of the invention. In this regard, no attempt is made to show structural details of the invention in more detail than is necessary for the fundamental understanding of the invention, the description taken with the drawings and/or examples making apparent to those skilled in the art how the several forms of the invention may be embodied in practice.

Definitions and explanations used in the present disclosure are meant and intended to be controlling in any future construction unless clearly and unambiguously modified in the examples or when application of the meaning renders any construction meaningless or essentially meaningless. In cases where the construction of the term would render it meaningless or essentially meaningless, the definition should be taken from Webster's Dictionary, 3rd Edition or a dictionary known to those of ordinary skill in the art, such as the Oxford Dictionary of Biochemistry and Molecular Biology (Eds. Attwood T et al., Oxford University Press, Oxford, 2006).

What is claimed is:

1. A nucleic acid comprising a recombinant receptor coding sequence, wherein the recombinant receptor, when expressed by a cell, comprises
an extracellular component comprising
a binding domain comprising
a variable heavy chain with a complementarity determining region (CDRH) 1 as set forth in SEQ ID NO: 2, a CDRH2 as set forth in SEQ ID NO: 3, and a CDRH3 as set forth in SEQ ID NO: 4, and
a variable light chain complementarity determining region (CDRL) 1 as set forth in SEQ ID NO: 5, a CDRL2 comprising the sequence AAS, and a CDRL3 as set forth in SEQ ID NO: 7; and
a spacer comprising an IgG4 hinge domain;
an intracellular component comprising an effector domain; and
a transmembrane domain linking the extracellular component to the intracellular component.

2. The nucleic acid of claim 1, wherein the intracellular component comprises a CD3ζ signaling domain and a 41-BB co-stimulatory domain.

3. The nucleic acid of claim 2, wherein the CD3ζ signaling domain is encoded by the sequence as set forth in SEQ ID NO: 23.

4. The nucleic acid of claim 2, wherein the 41-BB co-stimulatory domain is encoded by the sequence as set forth in SEQ ID NO: 21.

5. A nucleic acid encoding a recombinant receptor, wherein the recombinant receptor, when expressed by a cell, comprises
an extracellular component comprising a binding domain that binds a B-cell activating factor receptor (BAFF-R);
an intracellular component comprising an effector domain; and
a transmembrane domain linking the extracellular component to the intracellular component.

6. The nucleic acid of claim 5, wherein the encoded recombinant receptor comprises a chimeric antigen receptor (CAR), an engineered T cell receptor, or a hybrid thereof.

7. The nucleic acid of claim 5, wherein the encoded recombinant receptor comprises a CAR.

8. The nucleic acid of claim 5, wherein the binding domain is derived from an H90 monoclonal antibody.

9. The nucleic acid of claim 5, wherein the binding domain comprises: a variable heavy chain with a complementarity determining region (CDRH) 1 having at least 95% sequence identity to the sequence as set forth in SEQ ID NO: 2, a CDRH2 having at least 95% sequence identity to the sequence as set forth in SEQ ID NO: 3, and a CDRH3 having at least 95% sequence identity to the sequence as set forth in SEQ ID NO: 4 or a sequence having 0-5 conservative amino acid substitutions thereof; and
a variable light chain complementarity determining region (CDRL) 1 having at least 95% sequence identity to the sequence as set forth in SEQ ID NO: 5, a CDRL2 having at least 95% sequence identity to the sequence AAS, and a CDRL3 having at least 95% sequence identity to the sequence as set forth in SEQ ID NO: 7 or a sequence having 0-5 conservative amino acid substitutions thereof.

10. The nucleic acid of claim 5, wherein the binding domain comprises: a variable heavy chain with complementarity determining regions (CDRH) 1 as set forth in SEQ ID NO: 2, a CDRH2 as set forth in SEQ ID NO: 3, and a CDRH3 as set forth in SEQ ID NO: 4, and a variable light chain complementarity determining region (CDRL) 1 as set forth in SEQ ID NO: 5, a CDRL2 comprising the sequence AAS, and a CDRL3 as set forth in SEQ ID NO: 7.

11. The nucleic acid of claim 5, wherein the binding domain comprises a variable heavy chain having at least 95% sequence identity to the sequence as set forth in SEQ ID NO: 8 or a sequence having 0-10 conservative amino acid substitutions thereof; and a variable light chain having at least 95% sequence identity to the sequence set forth in SEQ ID NO: 9 or a sequence having 0-10 conservative amino acid substitutions thereof.

12. The nucleic acid of claim 5, wherein the binding domain comprises a variable heavy chain as set forth in SEQ ID NO: 8 and a variable light chain set forth in SEQ ID NO: 9.

13. The nucleic acid of claim 5, wherein the binding domain comprises a humanized amino acid sequence.

14. The nucleic acid of claim 5, wherein the binding domain comprises a humanized VH domain comprising a sequence having at least 95% sequence identity to the sequence as set forth in SEQ ID NO: 32, SEQ ID NO: 8, or SEQ ID NO: 34; and a humanized VL domain comprising a sequence having at least 95% sequence identity to the sequence as set forth in SEQ ID NO: 35, SEQ ID NO: 9, or SEQ ID NO: 37.

15. The nucleic acid of claim 5, wherein the binding domain comprises a humanized VH domain comprising a sequence as set forth in SEQ ID NO: 32, SEQ ID NO: 8, or SEQ ID NO: 34; and a humanized VL domain comprising a sequence as set forth in SEQ ID NO: 35, SEQ ID NO: 9, or SEQ ID NO: 37.

16. The nucleic acid of claim 5, wherein the binding domain comprises an scFv.

17. The nucleic acid of claim 16, wherein the scFv has at least 95% sequence identity to the sequence as set forth in SEQ ID NO: 1 or SEQ ID NO: 6.

18. The nucleic acid of claim 16, wherein the scFv has 1-10 conservative amino acid substitutions as compared to the sequence as set forth in SEQ ID NO: 1 or SEQ ID NO: 6.

19. The nucleic acid of claim 16, wherein the scFv comprises the sequence as set forth in SEQ ID NO: 1 or SEQ ID NO: 6.

20. The nucleic acid of claim 5, wherein the extracellular component further comprises a spacer.

21. The nucleic acid of claim 20, wherein the spacer comprises a long spacer, medium spacer, or short spacer.

22. The nucleic acid of claim 20, wherein the spacer has a length of 10-15 residues, 110-130 residues, or 230-240 residues.

23. The nucleic acid of claim 21, wherein the short spacer has a length of 12 residues.

24. The nucleic acid of claim 21, wherein the medium spacer has a length of 119 residues.

25. The nucleic acid of claim 21, wherein the long spacer has a length of 229 residues.

26. The nucleic acid of claim 20, wherein the spacer comprises an IgG4 hinge domain.

27. The nucleic acid of claim 20, wherein the spacer further comprises an IgG4 CH3 domain.

28. The nucleic acid of claim 20, wherein the spacer further comprises an IgG4 CH2 domain.

29. The nucleic acid of claim 20, wherein the spacer comprises a CD8a hinge domain.

30. The nucleic acid of claim 20, wherein the spacer comprises a sequence having at least 95% sequence identity to the sequence as set forth in SEQ ID NO: 15, SEQ ID NO: 13, SEQ ID NO: 11, or SEQ ID NO: 70, or a sequence having 0-3 conservative amino acid substitutions thereof.

31. The nucleic acid of claim 20, wherein the spacer comprises a sequence as set forth in SEQ ID NO: 15, SEQ ID NO: 13, SEQ ID NO: 11, or SEQ ID NO: 70.

32. The nucleic acid of claim 5, wherein the effector domain comprises all or a portion of the signaling domain of CD3ζ, CD27, CD28, 4-1BB, OX-40, CD30, CD40, PD-1, ICOS, LFA-1, CD2, CD7, NKG2C, and/or B7-H3.

33. The nucleic acid of claim 5, wherein the effector domain comprises all or a portion of the signaling domain of CD3ζ and 4-1BB.

34. The nucleic acid of claim 33, wherein the signaling domain of CD3ζ comprises a sequence having at least 95% sequence identity to the sequence as set forth in SEQ ID NO: 22.

35. The nucleic acid of claim 33, wherein the signaling domain of CD3ζ has a sequence having 1-5 conservative amino acid substitutions as compared to SEQ ID NO: 22.

36. The nucleic acid of claim 33, wherein the signaling domain of CD34 comprises a sequence as set forth in SEQ ID NO: 22.

37. The nucleic acid of claim 33, wherein the signaling domain of 4-1BB comprises a sequence having at least 95% sequence identity to the sequence as set forth in SEQ ID NO: 20.

38. The nucleic acid of claim 33, wherein the signaling domain of 4-1BB has 1-5 conservative amino acid substitutions as compared to SEQ ID NO: 20.

39. The nucleic acid of claim 33, wherein the signaling domain of 4-1BB comprises a sequence as set forth in SEQ ID NO: 20.

40. The nucleic acid of claim 5, wherein the transmembrane domain comprises a CD28 transmembrane domain.

41. The nucleic acid of claim 40, wherein the CD28 transmembrane domain comprises a sequence having at least 95% sequence identity to the amino acid sequence of SEQ ID NO: 17.

42. The nucleic acid of claim 40, wherein the CD28 transmembrane domain has 1-3 conservative amino acid substitutions as compared to SEQ ID NO: 17.

43. The nucleic acid of claim 40, wherein the CD28 transmembrane domain comprises a sequence as set forth in SEQ ID NO: 17.

44. The nucleic acid of claim 5, wherein the transmembrane domain comprises a CD8a transmembrane domain.

45. The nucleic acid of claim 44, wherein the CD8a transmembrane domain comprises a sequence having at least 95% sequence identity to the amino acid sequence of SEQ ID NO: 71.

46. The nucleic acid of claim 44, wherein the CD8a transmembrane domain has 1-3 conservative amino acid substitutions as compared to SEQ ID NO: 71.

47. The nucleic acid of claim 44, wherein the CD8a transmembrane domain comprises a sequence as set forth in SEQ ID NO: 71.

48. The nucleic acid of claim 5, further comprising or encoding a control feature selected from a tag cassette, a transduction marker, a selection cassette, and/or a suicide switch.

49. The nucleic acid of claim 48, wherein the transduction marker comprises a truncated EGFR (EGFRt) polypeptide or a truncated Her2 (Her2t).

50. The nucleic acid of claim 48, wherein the transduction marker comprises a sequence having at least 95% sequence identity to the sequence as set forth in SEQ ID NO: 29.

51. The nucleic acid of claim 48, wherein the transduction marker has 1-5 conservative amino acid substitutions as compared to SEQ ID NO: 29.

52. The nucleic acid of claim 48, wherein the transduction marker comprises a sequence as set forth in SEQ ID NO: 29.

53. The nucleic acid of claim 48, having the transduction marker encoding sequence as set forth in SEQ ID NO: 30.

54. The nucleic acid of claim 48, wherein the transduction marker comprises a sequence having at least 95% sequence identity to the sequence as set forth in SEQ ID NO: 85.

55. The nucleic acid of claim 48, wherein the transduction marker has 1-5 conservative amino acid substitutions as compared to SEQ ID NO: 85.

56. The nucleic acid of claim 48, wherein the transduction marker comprises a sequence as set forth in SEQ ID NO: 85.

57. The nucleic acid of claim 48, having the transduction marker encoding sequence as set forth in SEQ ID NO: 86.

58. The nucleic acid of claim 48, wherein the selection cassette comprises a dihydrofolate reductase double mutant (DHFRdm).

59. The nucleic acid of claim 58, wherein the DHFRdm comprises a sequence having at least 95% sequence identity to the sequence as set forth in SEQ ID NO: 26.

60. The nucleic acid of claim 58, wherein the DHFRdm has 1-5 conservative amino acid substitutions as compared to SEQ ID NO: 26.

61. The nucleic acid of claim 58, wherein the DHFRdm comprises a sequence as set forth in SEQ ID NO: 26.

62. The nucleic acid of claim 48, wherein the suicide switch comprises herpes simplex virus thymidine kinase/ganciclovir (HSVTK/GCV) suicide gene system, or an inducible caspase suicide gene system.

63. The nucleic acid of claim 5, further comprising a ribosomal skip element.

64. The nucleic acid of claim 63, wherein the ribosomal skip element comprises T2A, P2A, E2A, or F2A.

65. The nucleic acid of claim 63, wherein the ribosomal skip element comprises T2A.

66. The nucleic acid of claim 63, wherein the ribosomal skip element comprises P2A.

67. The nucleic acid sequence of claim 5, further comprising a promoter operably linked to the coding sequence encoding the recombinant receptor.

68. The nucleic acid sequence of claim 67, wherein the promoter comprises a constitutive promoter.

69. The nucleic acid of claim 68, wherein the constitutive promoter comprises an EF1a promoter.

70. The nucleic acid of claim 5, further comprising an inducible promoter operably linked to the coding sequence encoding the recombinant receptor.

71. The nucleic acid of claim 5, wherein the nucleic acid comprises cDNA.

72. A nanoparticle encapsulating the nucleic acid of claim 5.

73. A vector comprising the nucleic acid claim 5.

74. The vector of claim 73, wherein the vector comprises a viral vector, a transposon vector, an integrase vector, or an mRNA vector.

75. The vector of claim 74, wherein the viral vector comprises a lentiviral vector, a foamy viral vector, a retroviral vector, or a gamma retroviral vector.

76. The vector of claim 74, wherein the viral vector comprises a lentiviral vector.

77. Use of the nanoparticle of claim 72 or the vector of claim 73 to treat a subject in need thereof, wherein the subject has low BAFF-R antigen cell density.

78. A cell genetically modified to comprise the nucleic acid of claim 5.

79. The cell of claim 78, further comprising a genetic construct encoding a second recombinant receptor.

80. The cell of claim 79, wherein the second recombinant receptor comprises an extracellular domain comprising a binding domain that binds a cancer antigen, a B-cell specific ligand, and/or a small molecule.

81. The cell of claim 80, wherein the cancer antigen comprises EGFR, B7H3, CD171, ROR1, HER2, IL-13Rα2, GD2, CA-125, MUC-1, EphA2, MAGE-A3, MAGE-A4, MAGE-C2, PRAME, SSX2, adipophilin, AIM2, ALDH1A1, BCLX, EpCAM, CS274, CPSF, cyclin D1, DKK1, ENAH, EPHA3, EZH2, FGF5, G250, HLA-DOB, ID01, IGF2B3, KIF20A, M-CSF, MCSP, MDM2, Meloe, MMP-2, MMP-7, MUC1, MUC5AC, p53, PAX5, PBF, PRAME, PSMA, RAGE-1, RGS5, RhoC, RNF43, RUF43, FU2AS, SOX10, STEAP1, TPBG, VEGF, WT1, or NY-ESO-1.

82. The cell of claim 80, wherein the B-cell specific ligand comprises EGFR, B7H3, CD171, ROR1, or IL-13Rα2.

83. The cell of claim 80, wherein the B-cell specific ligand comprises CD19, CD20, CD22, CD1d, CD5, CD21, CD23, CD24, CD25, CD27, CD32, CD34, CD35, CD38, CD40, CD44, CD45, CD45.1, CD45.2, CD54, CD69, CD72, CD79, CD80, CD84, LFA-1, CALLA, BCMA, B220 isoform of CD45, CD93, CD84, CD86, TNFSF7, TNFRSF5, ENPP-1, HVEM, BLIMP1, CXCR4, CD148, or CD147.

84. The cell of claim 80, wherein the B-cell specific ligand comprises CD19, CD20, or CD22.

85. The cell of claim 80, wherein the small molecule comprises fluorescein, dinitrophenol, biotin, folate, or a derivative thereof.

86. The cell of claim 80, wherein the extracellular domain comprises a spacer.

87. The cell of claim 86, wherein the spacer comprises a long spacer, medium spacer, or short spacer.

88. The cell of claim 86, wherein the spacer has a length of 10-15 residues, 110-130 residues, or 230-240 residues.

89. The cell of claim 87, wherein the short spacer has a length of 12 residues.

90. The cell of claim 87, wherein the medium spacer has a length of 119 residues.

91. The cell of claim 87, wherein the long spacer has a length of 229 residues.

92. The cell of claim 86, wherein the spacer comprises an IgG4 hinge domain.

93. The cell of claim 86, wherein the spacer further comprises an IgG4 CH3 domain.

94. The cell of claim 86, wherein the spacer further comprises an IgG4 CH2 domain.

95. The cell of claim 86, wherein the spacer comprises a CD8a hinge domain.

96. The cell of claim 86, wherein the spacer comprises a sequence having at least 95% sequence identity to the sequence as set forth in SEQ ID NO: 15, SEQ ID NO: 13, SEQ ID NO: 11, or SEQ ID NO: 70, or a sequence having 0-3 conservative amino acid substitutions thereof.

97. The cell of claim 86, wherein the spacer comprises a sequence as set forth in SEQ ID NO: 15, SEQ ID NO: 13, SEQ ID NO: 11, or SEQ ID NO: 70.

98. The cell of claim 78, wherein the cell is an autologous cell or an allogeneic cell in reference to a subject.

99. The cell of claim 78, wherein the cell is a T cell, B cell, natural killer (NK) cell, NK-T cell, monocyte/macrophage, hematopoietic stem cells (HSC), or a hematopoietic progenitor cell (HPC).

100. The cell of claim 78, wherein the cell is a T cell selected from a CD3+ T cell, a CD4+ T cell, a CD8+ T cell, a central memory T cell, an effector memory T cell, and/or a naïve T cell.

101. The cell of claim 78, wherein the cell is a CD8+ T cell and/or a CD4+ T cell.

102. The cell of claim 78, wherein the cell is in vivo, in vitro, or ex vivo.

103. The cell of claim 78, wherein the cell is mammalian.

104. The cell of claim 78, wherein the cell is human.

105. A formulation comprising the cell of claim 78 and a pharmaceutically acceptable carrier.

106. A composition comprising i) the nucleic acid of claim 5, the nanoparticle of claim 72, or the vector of claim 73; and ii) a pharmaceutically acceptable carrier.

107. A method of treating a subject in need thereof comprising administering a therapeutically effective amount of the formulation of claim 105 and/or the composition of claim 106 to the subject thereby treating the subject in need thereof.

108. The method of claim 107, wherein the subject in need thereof has cancer.

109. The method of claim 108, wherein the cancer comprises a BAFF-R+ cell.

110. The method of claim 108, wherein the cancer comprises a B cell cancer.

111. The method of claim 108, wherein cancer comprises mantle cell lymphoma (MCL), multiple myeloma (MM), acute lymphoblastic leukemia (ALL), and diffuse large B-cell lymphoma (DLBCL).

112. The method of claim 107, wherein the formulation comprises autologous cells or allogeneic cells.

113. The method of claim 107, wherein the subject is mammalian.

114. The method of claim 107, wherein the subject is human.

115. The method of claim 107, wherein the subject in need thereof has low BAFF-R antigen cell density.

116. The method of claim 115, wherein the low BAFF-R antigen cell density comprises less than 50,000 BAFF-R molecules per diseased cell.

117. The method of claim 115, wherein the low BAFF-R antigen cell density comprises less than 30,000 BAFF-R molecules per diseased cell.

118. The method of claim 115, wherein the low BAFF-R antigen cell density comprises less than 15,000 BAFF-R molecules per diseased cell.

119. The method of claim 115, wherein the low BAFF-R antigen cell density comprises less than 10,000 BAFF-R molecules per diseased cell.

120. The method of claim 115, wherein the low BAFF-R antigen cell density comprises less than 7,000 BAFF-R molecules per diseased cell.

121. The method of claim 107, further comprising obtaining a sample of the diseased cells and measuring the BAFF-R antigen cell density.

122. Use of the cell of claim 78 in the manufacture of a medicament for treating, inhibiting or ameliorating a disorder in a subject.

123. Use of the cell of claim 78, to treat a subject in need thereof, wherein the subject has low BAFF-R antigen cell density.

124. The cell of claim 123 for use as a medicament.

125. A method of genetically modifying a cell comprising introducing the nucleic acid of claim 5, the nanoparticle of claim 72, the vector of claim 73, or the composition of claim 106 into the cell.

126. The method of claim 125, further comprising stimulating the cell by contacting the cell with a cytokine.

127. The method of claim 126, wherein the cytokine is selected from IL-2, IL-7, IL-15, or IL-21.

128. The method of claim 126, wherein the stimulating is performed prior to the introducing.

129. The method of claim 126, further comprising initiating the cell by contacting the cell with an anti-CD3 antibody or antigen binding fragment thereof, and/or an anti-CD28 antibody or antigen binding fragment thereof

130. The method of claim 129, wherein the initiating is performed prior to the introducing.

131. The method of claim 126, further comprising culturing the cell to obtain a plurality of the cell in an amount sufficient to administer an immunotherapy to a subject.

132. The method of claim 126, wherein the cell is an autologous cell or an allogeneic cell in reference to a subject.

133. The method of claim 126, wherein the cell is a T cell, B cell, natural killer (NK) cell, NK-T cell, monocyte/macrophage, hematopoietic stem cells (HSC), or a hematopoietic progenitor cell (HPC).

134. The method of claim 126, wherein the cell is a T cell selected from a CD3+ T cell, a CD4+ T cell, a CD8+ T cell, a central memory T cell, an effector memory T cell, and/or a naïve T cell.

135. The method of claim 126, wherein the cell is a CD8+ T cell and/or a CD4+ T cell.

136. The method of claim 126, wherein the cell is in vivo, in vitro, or ex vivo.

137. The method of claim 126, wherein the cell is mammalian.

138. The method of claim 126, wherein the cell is human.

139. A composition comprising: a recombinant receptor encoded by the nucleic acid of claim 5, wherein the recombinant receptor is specifically bound to a BAFF-R polypeptide or fragment thereof.

140. The composition of claim 139, wherein an effector cell comprises the recombinant receptor.

141. The composition of claim 140, wherein the effector cell is a T cell, a CD4+ T cell, CD8+ T cell, a precursor T-cell, or a hematopoietic stem cell.

142. The composition of claim 139, wherein a target cell comprises the BAFF-R polypeptide or fragment thereof.

143. The composition of claim 142, wherein the target cell is a B cell.

144. The composition of claim 140, wherein the effector cell further comprises a transduction marker.

145. The composition of claim 144, wherein the transduction marker comprises a truncated EGFR (EGFRt) polypeptide or a truncated Her2 (Her2t).

146. The composition of claim 144, further comprising an antibody or antigen binding fragment thereof specifically bound to the transduction marker.

147. The composition of claim 146, wherein the antibody or antigen binding fragment thereof further comprises a detectable label or cytotoxic agent.


SEQUENCE LISTING (86 sequences)

SEQ ID NO: 1 - protein, 244 aa, Synthetic construct
VQLQESGPGLVKPSQTLSLTCTVSGDSITSGYWNWIRQHPGKGLEYIGYISYSGSTYYNPSLKSRVTISRDTSKNQYSLKLSSVTAADTAVYYCASPNYPFYAMDYWGQGTLVTVSSGGGGSGGGGSGGGGSDIVLTQSPATLSLSPGERATLSCRASESVDNYGISFMNWFQQKPGQAPRLLIYAASNRATGIPARFSGSGSGTDFTLTISSLEPEDFAVYYCQQSKEVPWTFGGGTKVEIKR

SEQ ID NO: 2 - protein, 8 aa, Synthetic construct
GDSITSGY

SEQ ID NO: 3 - protein, 7 aa, Synthetic construct
ISYSGST

SEQ ID NO: 4 - protein, 12 aa, Synthetic construct
ASPNYPFYAMDY

SEQ ID NO: 5 - protein, 10 aa, Synthetic construct
ESVDNYGISF

SEQ ID NO: 6 - protein, 244 aa, Synthetic construct
DIVLTQSPATLSLSPGERATLSCRASESVDNYGISFMNWFQQKPGQAPRLLIYAASNRATGIPARFSGSGSGTDFTLTISSLEPEDFAVYYCQQSKEVPWTFGGGTKVEIKRGGGGSGGGGSGGGGSVQLQESGPGLVKPSQTLSLTCTVSGDSITSGYWNWIRQHPGKGLEYIGYISYSGSTYYNPSLKSRVTISRDTSKNQYSLKLSSVTAADTAVYYCASPNYPFYAMDYWGQGTLVTVSS

SEQ ID NO: 7 - protein, 9 aa, Synthetic construct
QQSKEVPWT

SEQ ID NO: 8 - protein, 117 aa, Synthetic construct
VQLQESGPGLVKPSQTLSLTCTVSGDSITSGYWNWIRQHPGKGLEYIGYISYSGSTYYNPSLKSRVTISRDTSKNQYSLKLSSVTAADTAVYYCASPNYPFYAMDYWGQGTLVTVSS

SEQ ID NO: 9 - protein, 112 aa, Synthetic construct
DIVLTQSPATLSLSPGERATLSCRASESVDNYGISFMNWFQQKPGQAPRLLIYAASNRATGIPARFSGSGSGTDFTLTISSLEPEDFAVYYCQQSKEVPWTFGGGTKVEIKR

SEQ ID NO: 10 - protein, 15 aa, Synthetic construct
GGGGSGGGGSGGGGS

SEQ ID NO: 11 - protein, 229 aa, Synthetic construct
ESKYGPPCPPCPAPEFDGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 12 - DNA, 673 bp, Synthetic construct
  source 1..673: mol_type = other DNA
gaatctaagtacggaccgccctgcccccgagttcgacggcggacccagcgtgttcctgttcccccccaagcccaaggacaccctgatgatcagccggacccccgaggtgacctgcgtggtggtggacgtgagccaggaagatcccgaggtccagttcaattggtacgtggacggcgtggaagtgcacaacgccaagaccaagcccagagaggaacagttcaacagcacctaccgggtggtgtctgtgctgaccgtgctgcaccaggactggctgaacggcaaagaatacaagtgcaaggtgtccaacaagggcctgcccagcagcatcgaaaagaccatcagcaaggccaagggccagcctcgcgagccccaggtgtacaccctgcctccctcccaggaagagatgaccaagaaccaggtgtccctgacctgcctggtgaagggcttctaccccagcgacatcgccgtggagtgggagagcaacggccagcctgagaacaactacaagaccacccctcccgtgctggacagcgacggcagcttcttcctgtacagccggctgaccgtggacaagagccggtggcaggaaggcaacgtctttagctgcagcgtgatgcacgaggccctgcacaaccactacacccagaagagcctgagcctgtccctgggcaag

SEQ ID NO: 13 - protein, 119 aa, Synthetic construct
ESKYGPPCPPCPGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 14 - DNA, 357 bp, Synthetic construct
  source 1..357: mol_type = other DNA
gaatctaagtacggaccgccctgccccccttgccctggccagcctagagaaccccaggtgtacaccctgcctcccagccaggaagagatgaccaagaaccaggtgtccctgacctgcctggtcaaaggcttctaccccagcgatatcgccgtggaatgggagagcaacggccagcccgagaacaactacaagaccaccccccctgtgctggacagcgacggcagcttcttcctgtactcccggctgaccgtggacaagagccggtggcaggaaggcaacgtcttcagctgcagcgtgatgcacgaggccctgcacaaccactacacccagaagtccctgagcctgagcctgggcaag

SEQ ID NO: 15 - protein, 12 aa, Synthetic construct
ESKYGPPCPPCP

SEQ ID NO: 16 - DNA, 36 bp, Synthetic construct
  source 1..36: mol_type = other DNA
gaatctaagtacggaccgccctgccccccttgccct

SEQ ID NO: 17 - protein, 28 aa, Synthetic construct
MFWVLVVVGGVLACYSLLVTVAFIIFWV

SEQ ID NO: 18 - DNA, 84 bp, Synthetic construct
  source 1..84: mol_type = other DNA
atgttctgggtgctggtggtggtcggaggcgtgctggcctgctacagcctgctggtcaccgtggccttcatcatcttttgggtg

SEQ ID NO: 19 - protein, 72 aa, Synthetic construct
AAATTTPAPRPPTPAPTIASQPLSLRPEACRPAAGGAVHTRGLDFACDIYIWAPLAGTCGVLLLSLVITLYC

SEQ ID NO: 20 - protein, 42 aa, Synthetic construct
KRGRKKLLYIFKQPFMRPVQTTQEEDGCSCRFPEEEEGGCEL

SEQ ID NO: 21 - DNA, 126 bp, Synthetic construct
  source 1..126: mol_type = other DNA
aaacggggcagaaagaaactcctgtatatattcaaacaaccatttatgagaccagtacaaactactcaagaggaagatggctgtagctgccgatttccagaagaagaagaaggaggatgtgaactg

SEQ ID NO: 22 - protein, 112 aa, Synthetic construct
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 23 - DNA, 336 bp, Synthetic construct
  source 1..336: mol_type = other DNA
cgggtgaagttcagcagaagcgccgacgcccctgcctaccagcagggccagaatcagctgtacaacgagctgaacctgggcagaagggaagagtacgacgtcctggataagcggagaggccgggaccctgagatgggcggcaagcctcggcggaagaacccccaggaaggcctgtataacgaactgcagaaagacaagatggccgaggcctacagcgagatcggcatgaagggcgagcggaggcggggcaagggccacgacggcctgtatcagggcctgtccaccgccaccaaggatacctacgacgccctgcacatgcaggccctgcccccaagg

SEQ ID NO: 24 - protein, 154 aa, Synthetic construct
KRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 25 - protein, 22 aa, Synthetic construct
GSGATNFSLLKQAGDVEENPGP

SEQ ID NO: 26 - protein, 187 aa, Synthetic construct
MVGSLNCIVAVSQNMGIGKNGDFPWPPLRNESRYFQRMTTTSSVEGKQNLVIMGKKTWFSIPEKNRPLKGRINLVLSRELKEPPQGAHFLSRSLDDALKLTEQPELANKVDMVWIVGGSSVYKEAMNHPGHLKLFVTRIMQDFESDTFFPEIDLEKYKLLPEYPGVLSDVQEEKGIKYKFEVYEKND

SEQ ID NO: 27 - protein, 21 aa, Synthetic construct
GGGEGRGSLLTCGDVEENPGP

SEQ ID NO: 28 - DNA, 72 bp, Synthetic construct
  source 1..72: mol_type = other DNA
ctcgagggcggcggagagggcagaggaagtcttctaacatgcggtgacgtggaggagaatcccggccctagg

SEQ ID NO: 29 - protein, 335 aa, Synthetic construct
RKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSIATGMVGALLLLLVVALGIGLFM

SEQ ID NO: 30 - DNA, 1005 bp, Synthetic construct
  source 1..1005: mol_type = other DNA
cgcaaagtgtgtaacggaataggtattggtgaatttaaagactcactctccataaatgctacgaatattaaacacttcaaaaactgcacctccatcagtggcgatctccacatcctgccggtggcatttaggggtgactccttcacacatactcctcctctggatccacaggaactggatattctgaaaaccgtaaaggaaatcacagggtttttgctgattcaggcttggcctgaaaacaggacggacctccatgcctttgagaacctagaaatcatacgcggcaggaccaagcaacatggtcagttttctcttgcagtcgtcagcctgaacataacatccttgggattacgctccctcaaggagataagtgatggagatgtgataatttcaggaaacaaaaatttgtgctatgcaaatacaataaactggaaaaaactgtttgggacctccggtcagaaaaccaaaattataagcaacagaggtgaaaacagctgcaaggccacaggccaggtctgccatgccttgtgctcccccgagggctgctggggcccggagcccagggactgcgtctcttgccggaatgtcagccgaggcagggaatgcgtggacaagtgcaaccttctggagggtgagccaagggagtttgtggagaactctgagtgcatacagtgccacccagagtgcctgcctcaggccatgaacatcacctgcacaggacggggaccagacaactgtatccagtgtgcccactacattgacggcccccactgcgtcaagacctgcccggcaggagtcatgggagaaaacaacaccctggtctggaagtacgcagacgccggccatgtgtgccacctgtgccatccaaactgcacctacggatgcactgggccaggtcttgaaggctgtccaacgaatgggcctaagatcccgtccatcgccactgggatggtgggggccctcctcttgctgctggtggtggccctggggatcggcctcttcatg

SEQ ID NO: 31 - protein, 22 aa, Synthetic construct
MLLLVTSLLLCELPHPAFLLIP

SEQ ID NO: 32 - protein, 117 aa, Synthetic construct
VQLQESGPGLVKPSQTLSLTCTVSGDSITSGYWNWIRQHPGKGLEYIGYISYSGSTYYNPSLKSRVTISRDTSKNQFSLKLSSVTAADTAVYYCASPNYPFYAMDYWGQGTLVTVSS

SEQ ID NO: 33 - skipped

SEQ ID NO: 34 - protein, 117 aa, Synthetic construct
VQLQESGPGLVKPSETLSLTCSVSGDSITSGYWNWIRQPPGKGLEYIGYISYSGSTYYNPSLKSRVTISRDTSKNQYSLRLSSVTAADTALYYCASPNYPFYAMDYWGQGTRVTVSS

SEQ ID NO: 35 - protein, 112 aa, Synthetic construct
EIVLTQSPATLSLSPGERATLSCRASESVDNYGISFLNWFQQKPGQAPRLLIYAASNRATGIPARFSGSGSGTDFTLTISSLEPEDFAVYYCQQSKEVPWTFGGGTKVEIKR

SEQ ID NO: 36 - skipped

SEQ ID NO: 37 - protein, 112 aa, Synthetic construct
DIVMTQSPSSLSASVGDRVTITCRASESVDNYGISFMNWFQQKPGKAPKLLIYAASNLGSGVPSRFSGSGSGTDFTLTISSLQPEDFATYYCQQSKEVPWTFGQGTKVEIKR

SEQ ID NO: 38 - protein, 5 aa, Synthetic construct
  REPEAT 1..5: note = Resdiues 1-5 are repeated n times, wherein n is an integer of 1, 2, 3, 4, 5, 6, 7, 8, 9 or 10
GGGGS

SEQ ID NO: 39 - protein, 9 aa, Synthetic construct
  REPEAT 1..4: note = Residues 1-4 are repeated n times, wherein n is an integer of 1, 2, 3, 4, 5, 6, 7, 8, 9 or 10
  REPEAT 5..9: note = Residues 5-9 are repeated n times, wherein n is an integer of 1, 2, 3, 4, 5, 6, 7, 8, 9 or 10
GGGSGGGGS

SEQ ID NO: 40 - protein, 7 aa, Synthetic construct
  REPEAT 1..4: note = Residues 1-4 are repeated n times, wherein n is an integer of 1, 2, 3, 4, 5, 6, 7, 8, 9 or 10
  REPEAT 5..7: note = Residues 5-7 are repeated n times, wherein n is an integer of 1, 2, 3, 4, 5, 6, 7, 8, 9 or 10
GGGSGGS

SEQ ID NO: 41 - protein, 9 aa, Synthetic construct
  REPEAT 1..4: note = Residues 1-4 are repeated n times, wherein n is an integer of 1, 2, 3, 4, 5, 6, 7, 8, 9 or 10
GGGSGGGGS

SEQ ID NO: 42 - protein, 20 aa, Synthetic construct
GGGGSGGGGSGGGGSGGGGS

SEQ ID NO: 43 - protein, 15 aa, Synthetic construct
GGGGSGGGGSGGGGS

SEQ ID NO: 44 - protein, 10 aa, Synthetic construct
GGGGSGGGGS

SEQ ID NO: 45 - protein, 5 aa, Synthetic construct
GGGGS

SEQ ID NO: 46 - protein, 8 aa, Synthetic construct
GGGSGGGS

SEQ ID NO: 47 - protein, 4 aa, Synthetic construct
GGGS

SEQ ID NO: 48 - protein, 6 aa, Synthetic construct
GGSGGS

SEQ ID NO: 49 - protein, 11 aa, Synthetic construct
GGSGGGSGGSG

SEQ ID NO: 50 - protein, 10 aa, Synthetic construct
GGSGGGSGSG

SEQ ID NO: 51 - protein, 8 aa, Synthetic construct
GGSGGGSG

SEQ ID NO: 52 - protein, 5 aa, Synthetic construct
  REPEAT 1..5: note = Residues 1-5 are repeated n times, wherein n is an integer including 1, 2, 3, 4, 5, 6, 7, 8, 9, or more.
EAAAK

SEQ ID NO: 53 - protein, 6 aa, Synthetic construct
HHHHHH

SEQ ID NO: 54 - protein, 8 aa, Synthetic construct
DYKDDDDK

SEQ ID NO: 55 - protein, 8 aa, Synthetic construct
DLYDDDDK

SEQ ID NO: 56 - protein, 15 aa, Synthetic construct
GLNDIFEAQKIEWHE

SEQ ID NO: 57 - protein, 26 aa, Synthetic construct
KRRWKKNFIAVSAANRFKKISSSGAL

SEQ ID NO: 58 - protein, 9 aa, Synthetic construct
YPYDVPDYA

SEQ ID NO: 59 - protein, 10 aa, Synthetic construct
EQKLISEEDL

SEQ ID NO: 60 - protein, 8 aa, Synthetic construct
WRHPQFGG

SEQ ID NO: 61 - protein, 8 aa, Synthetic construct
WSHPQFEK

SEQ ID NO: 62 - protein, 13 aa, Synthetic construct
SLAELLNAGLGGS

SEQ ID NO: 63 - protein, 8 aa, Synthetic construct
TQDPSRVG

SEQ ID NO: 64 - protein, 14 aa, Synthetic construct
GKPIPNPLLGLDST

SEQ ID NO: 65 - protein, 27 aa, Synthetic construct
MFWVLVVVGGVLACYSLLVTVAFIIFW

SEQ ID NO: 66 - protein, 26 aa, Synthetic construct
FWVLVVVGGVLACYSLLVTVAFIIFW

SEQ ID NO: 67 - DNA, 84 bp, Synthetic construct
  source 1..84: mol_type = other DNA
atgttctgggtgctggtggtggtgggcggggtgctggcctgctacagcctgctggtgacagtggccttcatcatcttttgggta

SEQ ID NO: 68 - DNA, 81 bp, Synthetic construct
  source 1..81: mol_type = other DNA
atgttctgggtgctggtggtggtcggaggcgtgctggcctgctacagcctgctggtcaccgtggccttcatcatcttttgg

SEQ ID NO: 69 - DNA, 81 bp, Synthetic construct
  source 1..81: mol_type = other DNA
atgttctgggtgctggtggtggtgggcggggtgctggcctgctacagcctgctggtgacagtggccttcatcatcttttgg

SEQ ID NO: 70 - protein, 48 aa, Synthetic construct
AAATTTPAPRPPTPAPTIASQPLSLRPEACRPAAGGAVHTRGLDFACD

SEQ ID NO: 71 - protein, 24 aa, Synthetic construct
IYIWAPLAGTCGVLLLSLVITLYC

SEQ ID NO: 72 - protein, 43 aa, Synthetic construct
VKRGRKKLLYIFKQPFMRPVQTTQEEDGCSCRFPEEEEGGCEL

SEQ ID NO: 73 - protein, 41 aa, Synthetic construct
VKRGRKKLLYIFKQPFMRPVQTTQEEDGCSCRFPEEEEGGC

SEQ ID NO: 74 - DNA, 126 bp, Synthetic construct
  source 1..126: mol_type = other DNA
aagagaggacggaagaagctgctgtatatctttaagcagccttttatgagacctgtccaaacaacccaagaggaggacgggtgttcctgtcgcttcccagaggaggaggaaggagggtgcgagctg

SEQ ID NO: 75 - DNA, 129 bp, Synthetic construct
  source 1..129: mol_type = other DNA
gtgaaacggggcagaaagaaactcctgtatatattcaaacaaccatttatgagaccagtacaaactactcaagaggaagatggctgtagctgccgatttccagaagaagaagaaggaggatgtgaactg

SEQ ID NO: 76 - protein, 115 aa, Synthetic construct
GGGRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 77 - protein, 114 aa, Synthetic construct
ELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 78 - DNA, 345 bp, Synthetic construct
  source 1..345: mol_type = other DNA
ggcggagggcgggtgaagttcagcagaagcgccgacgcccctgcctaccagcagggccagaatcagctgtacaacgagctgaacctgggcagaagggaagagtacgacgtcctggataagcggagaggccgggaccctgagatgggcggcaagcctcggcggaagaacccccaggaaggcctgtataacgaactgcagaaagacaagatggccgaggcctacagcgagatcggcatgaagggcgagcggaggcggggcaagggccacgacggcctgtatcagggcctgtccaccgccaccaaggatacctacgacgccctgcacatgcaggccctgcccccaagg

SEQ ID NO: 79 - protein, 24 aa, Synthetic construct
LEGGGEGRGSLLTCGDVEENPGPR

SEQ ID NO: 80 - protein, 21 aa, Synthetic construct
GSGEGRGSLLTCGDVEENPGP

SEQ ID NO: 81 - protein, 23 aa, Synthetic construct
GSGQCTNYALLKLAGDVESNPGP

SEQ ID NO: 82 - protein, 25 aa, Synthetic construct
GSGVKQTLNFDLLKLAGDVESNPGP

SEQ ID NO: 83 - DNA, 72 bp, Synthetic construct
  source 1..72: mol_type = other DNA
ctcgagggcggcggagagggcagaggaagtcttctaacatgcggtgacgtggaggagaatccaggccctagg

SEQ ID NO: 84 - protein, 357 aa, Synthetic construct
MLLLVTSLLLCELPHPAFLLIPRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSIATGMVGALLLLLVVALGIGLFM

SEQ ID NO: 85 - protein, 142 aa, Synthetic construct
MLLLVTSLLLCELPHPAFLLIPCHPECQPQNGSVTCFGPEADQCVACAHYKDPPFCVARCPSGVKPDLSYMPIWKFPDEEGACQPCPINCTHSCVDLDDKGCPAEQRASPLTGGGSGGGSIISAVVGILLVVVLGVVFGILI

SEQ ID NO: 86 - DNA, 426 bp, Synthetic construct
  source 1..426: mol_type = other DNA
atgcttctcctggtgacaagccttctgctctgtgagttaccacacccagcattcctcctgatcccatgccaccctgagtgtcagccccagaatggctcagtgacctgttttggaccggaggctgaccagtgtgtggcctgtgcccactataaggaccctcccttctgcgtggcccgctgccccagcggtgtgaaacctgacctctcctacatgcccatctggaagtttccagatgaggagggcgcatgccagccttgccccatcaactgcacccactcctgtgtggacctggatgacaagggctgccccgccgagcagagagccagcccgttaacgggtggaggcagcggaggtggctcgtagtagagacgccaccaaccgtaagacgaccagcaccagaacccccaccagaaaccctaggagtag